(19) United States
(12) Patent Application Publication
(10) Pub. No.: US 2025/0387517 A1
(43) Pub. Date: Dec. 25, 2025

(54) COMPOSITIONS FOR ACTIVATING AND SILENCING GENE EXPRESSION

(71) Applicant: The Board of Trustees of the Leland Stanford Junior University, Stanford, CA (US)

(72) Inventors: Nicole Delrosso, Stanford, CA (US); Adi Mukund, Stanford, CA (US); Peter Suzuki, Stanford, CA (US); Joshua Tycko, Stanford, CA (US); Michael C. Bassik, Stanford, CA (US); Lacramioara Bintu, Stanford, CA (US)

(21) Appl. No.: 18/843,896

(22) PCT Filed: Mar. 9, 2023
(86) PCT No.: PCT/US2023/064036

§ 371 Date: Sep. 4, 2024

Related U.S. Application Data
(60) Provisional application No. 63/318,144, filed on Mar. 9, 2022.

(51) Int. Cl. A61K 48/00; C12N 9/22; C12N 15/11; C12N 15/90

(52) U.S. Cl. CPC A61K 48/0066; A61K 48/0058; C12N 9/226; C12N 15/11; C12N 15/907; C12N 2310/20; C12N 2830/001

(57) ABSTRACT

Provided herein are compositions, systems, and kits comprising effector domains for activating and silencing gene expression. In particular, synthetic transcription factors comprising the effector domains are provided.

### CROSS-REFERENCE TO RELATED APPLICATIONS

This application claims the benefit of U.S. Provisional Application No. 63/318,144, filed Mar. 9, 2022, the content of which is herein incorporated by reference in its entirety.

### STATEMENT REGARDING FEDERALLY SPONSORED RESEARCH

This invention was made with Government support under contracts HG009436, HG011866, and GM128947 awarded by the National Institutes of Health. The Government has certain rights in the invention.

### FIELD

Provided herein are compositions, systems, and kits for activating and silencing gene expression. In particular, synthetic transcription factors comprising one or more of the effector domains and methods of using thereof are provided.

### SEQUENCE LISTING STATEMENT

The contents of the electronic sequence listing titled 40702_601_SequenceListing.xml (Size: 26,606,746 bytes; and Date of Creation: Mar. 8, 2023) is herein incorporated by reference in its entirety.

### BACKGROUND

Human gene expression is regulated by over two thousand transcription factors and chromatin regulators. Large scale efforts have mapped where in the human genome transcription factors (TFs) and chromatin regulators (CRs) bind. However, equivalent maps of transcriptional effector domains (EDs) are incomplete: ED annotations are currently missing for about 60% of human TFs. Moreover, the sequence characteristics of what makes a good human activation or repression domain are still under investigation.

Previous efforts to engineer synthetic transcription factors have pulled activation and repression domains from a small toolbox of previously discovered effector domains. One useful assay for characterizing individual EDs and testing specific sequence requirements consists of recruitment of domains and mutants to reporter genes. This approach has been extended from recruiting single domains to high-throughput assays in yeast, drosophila, and human cells with a subset of transcriptional domains or a subset of full length transcription factors. New methods are needed to identify new effector domains, including systematically mapping EDs across the thousands of human transcriptional proteins.

### SUMMARY

Provided herein are synthetic transcription factors comprising an effector domain. In some embodiments, the synthetic transcription factor comprises one or more activator domains, one or more repressor domains, or a combination thereof fused to a heterologous DNA binding domain.

In some embodiments, at least one of the one or more activator domains or at least one of the one or more repressor domains comprises an amino acid sequence having at least 70% (e.g., at least 75%, at least 80%, at least 85%, at least 90%, at least 95%, at least 98%, at least 99%, or 100%) identity to any of SEQ ID NOs: 1-12567 and 28214-28404. In some embodiments, at least one of the one or more activator domains or at least one of the one or more repressor domains comprises an amino acid sequence of any of SEQ ID NOs: 1-12567 and 28214-28404.

In some embodiments, at least one of the one or more activator domains or the one or more repressor domains comprises at least 10 contiguous amino acids of any of SEQ ID NOs: 1-12567 and 28214-28404.

In some embodiments, at least one of the one or more activator domains comprises an amino acid sequence having at least 70% identity to any of SEQ ID NOs: 31, 36, 111, 113, 153, 158, 165, 182, 184, 189, 224, 291, 311, 313, 352, 362, 367, 369, 375, 381, 407, 410, 415, 426, 430, 436, 472, 476, 478, 480, 483, 487-489, 494, 496, 498, 509, 512-517, 524, 526, 527, 530, 532, 533, 537, 541, 542, 545-547, 549, 552, 554, 557, 560-562, 565-568, 570-576, 578, 579, 580, 581, 582, 585, 587, 589, 590, 592, 595-598, 601, 603, 605, 607, 613, 617, 620, 622-624, 626, 627, 629, 630, 634-636, 639, 643, 646, 648, 651, 654, 658, 659, 662, 664, 666, 673, 675, 677, 678, 681, 684, 685, 686, 687, 689, 695, 696, 697, 699, 704, 705, 707-711, 713, 715, 716, 721, 723-725, 728, 729, 731-733, 735, 744, 746, 747, 753, 755, 760, 761, 764, 766-769, 773, and 775-984.

In some embodiments, at least one of the one or more activator domains comprises an amino acid sequence having at least 70% identity to any of SEQ ID NOs: 88, 144, 147, 148, 149, 234, 280, 281, 282, 283, 302, 306, 307, 322, 355, 356, 440, 441, 442, 443, 444, 445, 446, 447, 448, 449, 477, 488, 501, 532, 548, 593, 610, 618, 676, 738, 757, and 28365-28404.

In some embodiments, at least one of the one or more activator domains comprises an amino acid sequence having at least 70% identity to any of SEQ ID NOs: 12568-13273.

In some embodiments, at least one of the one or more activator domains comprises an amino acid sequence having at least 70% identity to any of SEQ ID NOs: 13274-17423.

In some embodiments, at least one of the one or more activator domains comprises one or more of SEQ ID NOs: 17424-17841.

In some embodiments, at least one of the one or more repressor domains comprises an amino acid sequence having at least 70% identity to any of SEQ ID NOs: 1036, 1054, 1055, 1069, 1120, 1144, 1182, 1183, 1200, 1208, 1314, 1318, 1366, 1402, 1417, 1442, 1516, 1518, 1543, 1598, 1627, 1655, 1665, 1667, 1670, 1706, 1710, 1711, 1735, 1738, 1742, 1747, 1748, 1752, 1756, 1763, 1777, 1783, 1786, 1789, 1793, 1794, 1808, 1811, 1822, 1831, 1838, 1839, 1854, 1859, 1862, 1865, 1866, 1869, 1870, 1872, 1875, 1883, 1889, 1891, 1893, 1901, 1902, 1905, 1907, 1910, 1912, 1913, 1914, 1915, 1916, 1922, 1923, 1927, 1930, 1934, 1940, 1944, 1946, 1948, 1951, 1952, 1956, 1957, 1968, 1969, 1972, 1987, 1992, 1994, 1996, 2004, 2007, 2010, 2017, 2022, 2029, 2033, 2041, 2042, 2043, 2048, 2050, 2051, 2053, 2057, 2064, 2095, 2107, 2112, 2119, 2123, 2128, 2131, 2139, 2150, 2157, 2160, 2163, 2176, 2182, 2188, 2190, 2192, 2193, 2194, 2205, 2206, 2207, 2208, 2211, 2212, 2213, 2216, 2218, 2221, 2224, 2227, 2231, 2232, 2239, 2245, 2246, 2254, 2262, 2263, 2265, 2271, 2274, 2275, 2277, 2278, 2282, 2283, 2288, 2292, 2295, 2296, 2298, 2302, 2312, 2313, 2316, 2320, 2321, 2323, 2324, 2325, 2334, 2338, 2341, 2348, 2361, 2364, 2365, and 2370-6094.

In some embodiments, at least one of the one or more repressor domains comprises an amino acid sequence having at least 70% identity to any of SEQ ID NOs: 985, 986, 1005, 1042, 1050, 1063, 1064, 1090, 1098, 1099, 1124, 1126, 1127, 1129, 1276, 1277, 1280, 1284, 1342, 1367, 1375, 1397, 1406, 1409, 1410, 1427, 1428, 1430, 1442, 1447, 1459, 1486, 1487, 1492, 1494, 1511, 1512, 1513, 1564, 1569, 1650, 1651, 1652, 1653, 1661, 1680, 1681, 1723, 1730, 1733, 1740, 1741, 1795, 1848, 1864, 1865, 1914, 1915, 1991, 1998, 2007, 2017, 2092, 2100, 2103, 2142, 2147, 2155, 2168, 2224, 2235, 2251, 2264, 2278, 2283, 2298, 2306, 2312, 2320, 2323, 2331, 2339, 2356, 2366, 2471, 2481, 2617, 2731, 3150, 3336, 3853, 4713, 4797, 5742, 5743, 5870, 5878, 5940, 5945, and 28214-28364.

In some embodiments, at least one of the one or more repressor domains comprises an amino acid sequence having at least 70% identity to any of SEQ ID NOs: 17842-24889.

In some embodiments, at least one of the one or more repressor domains comprises one or more of SEQ ID NOs: 24890-25651.

In some embodiments, the heterologous DNA binding domain is a programmable DNA binding domain. In some embodiments, the heterologous DNA binding domain is derived from a Clustered Regularly Interspaced Short Palindromic Repeats associated (Cas) protein.

In some embodiments, the heterologous DNA binding domain is derived from a Transcription activator-like effectors (TALEs) domain.

In some embodiments, the heterologous DNA binding domain is part of an inducible DNA binding system.

Also provided herein are nucleic acids and vectors encoding the synthetic transcription factors disclosed herein.

Further provided are cells comprising the synthetic transcription factor disclosed herein, or nucleic acids encoding the synthetic transcription factors. In some embodiments, the cell comprises two or more synthetic transcription factors, nucleic acids, or vectors. In some embodiments, the cell is a prokaryotic cell. In some embodiments, the cell is a eukaryotic cell. In some embodiments, the cell is a human cell.

Compositions and systems comprising a synthetic transcription factor disclosed herein, a nucleic acid encoding a synthetic transcription factor, or a cell comprising a synthetic transcription factor are further provided. In some embodiments, the composition or system comprises two or more synthetic transcription factors, nucleic acids, vectors, or cells. In some embodiments, the composition or system further comprises an exogenous factor for use with the DNA binding domain (e.g., a guide RNA or a nucleic acid encoding a guide RNA).

Additionally provided are methods of using the synthetic transcription factors disclosed herein, or nucleic acids encoding the synthetic transcription factors. In some embodiments, the methods comprise modulating the expression of at least one target gene in a cell comprising introducing into the cell at least one synthetic transcription factors disclosed herein, nucleic acid encoding at least one synthetic transcription factor, or a composition or system comprising thereof. In some embodiments, the at least one target gene is an endogenous gene, an exogenous gene, or a combination thereof. In some embodiments, the cell is in a subject. In some embodiments, the method comprises administering the at least one synthetic transcription factor, nucleic acid, vector, or composition or system to the subject. In some embodiments, the gene expression of at least two genes is modulated.

In some embodiments, the methods comprise treating a disease or condition in a subject in need thereof, the method comprising: administering to the subject at least one synthetic transcription factors disclosed herein, nucleic acid encoding at least one synthetic transcription factor, or a composition or system comprising thereof. In some embodiments, the subject is human. In some embodiments, the synthetic transcription factor alters the expression of a disease-related gene.

Other aspects and embodiments of the disclosure will be apparent in light of the following detailed description.

### BRIEF DESCRIPTION OF THE DRAWINGS

FIGS. 1A-1J show that a high-throughput tiling screen across 2,047 human transcription factors (TFs) and chromatin regulators (CRs) finds hundreds of effector domains. FIG. 1A is a schematic of HT-recruit. A pooled library of protein tiles is synthesized, cloned as a fusion to rTetR-3xFLAG, and delivered to reporter cells. The reporter includes fluorescent citrine and a synthetic surface marker for magnetic bead separation of ON and OFF cells. FIG. 1B is activation and repression enrichment scores for MYB. Each horizontal line is a tile, and each vertical bar is the range of measurements from 2 biologically independent screens. Dashed horizontal line is the hit calling threshold based on random controls. Points with larger marker sizes are hits in the validation screen. Marker hues indicate FLAG-stained expression levels. FIGS. 1C and 1D show the distribution of the strongest effector domains (Eds) from the top 40 gene families. Average enrichment scores are from the maximum tile within each domain measured in the validation screen (n=2). All points shown are above the hit thresholds. FIG. 1E is tiling results for BRD4, TET2, ARID3B, and ETV1 (n=2 screens, dots are the mean, vertical bars the range). FIG. 1F is citrine fluorescence distributions from flow cytometry for cell lines expressing individual activating tiles (n=2). Vertical line is the citrine gate used to determine the fraction of cells ON (written above each distribution). FIG. 1G is a comparison between screen measurements and individually recruited tiles at minCMV (n=2, dots are the mean, bars the range) with logistic model fit plotted as solid line (r²=0.67, n=23). Dashed line is the hits threshold. FIG. 1H is flow cytometry citrine distributions for individual validations of repressing tiles (n=2). FIG. 1I is a comparison between screen measurements and individually recruited tiles at pEF (n=2, dots are the mean, bars the range) with logistic model fit as solid line (r²=0.84, n=22). FIG. 1J is effector domain counts identified herein shown above the black line, and domain counts from prior work not tested herein shown below. Repression domains (RDs) are annotated from tiles that were hits in both pEF and PGK promoter screens (FIG. 8).

FIGS. 2A-2I show hydrophobic amino acids interspersed with acidic, serine, proline or glutamine residues facilitate activation domain (AD) activity. FIG. 2A shows the fraction of activating tiles that contain compositional biases. FIG. 2B is the enrichment ratio for each aa across all activating tiles. Dashed line is at 1. FIG. 2C is a deletion scan across ADs of NFAT5 (SEQ ID NO: 684). Yellow rectangle is WT enrichment score, its height the range of two biologically independent screens. Each horizontal line represents which residues were deleted, dots are the mean, vertical bars the range, and p-values less than 0.05 (one-sided z-test compared to WT) are labeled in grey as decrease. FIG. 2D shows counts of deletion sequences containing a homotypic repeat of 3 or more amino acids of the indicated type binned according to their effect compared to WT (Fisher's exact test compared with AAA+ and LLL+ distribution, two-sided, Ser p=5.1e-5, Pro=1.9e-2, acidic=1.2e-4, Gln=1.5e-2, Gly=2.3e-2). FIG. 2E is the distribution of average activation enrichment scores (n=2) for WT and W,F,Y,L mutant tiles for all well-expressed W,F,Y,L-containing activating tiles (Mann-Whitney one-sided U test, p=9.2e-241). Shown are SEQ ID NOs: 28199 and 28200. FIG. 2F is the distribution of average activation enrichment scores (n=2) for WT and D,E mutant tiles for all well-expressed D,E containing activating tiles (Mann-Whitney one-sided U test, p=2.6e-61). Shown are SEQ ID NOs: 28201 and 28202. FIG. 2G, top, is distributions of average activation enrichment scores (n=2) for WT (colors) and comp. bias mutants (gray). FIG. 2G, bottom, is mutant enrichment scores subtracted from WT plotted for each comp. bias that was replaced with Ala. Dashed line is 2 times the average standard deviation (across all mutants) above 0. Probability these distributions would be observed for L: 7.7e-19, D: 0.0006, E: 0.0005, S: 0.56, and P: 0.006 (Mann-Whitney one-sided U test). Shown are SEQ ID NOs: 28203 and 28204. FIG. 2H is counts of all regions within comp. biased tiles that lost activity upon mutation, colored by containing W, F, Y, L or not (Fisher's exact test, two-sided, compared to the same tiles' comp. biased sequences that had no change upon deletion, Ser: p=3.8e-4, acidic: p=3.0e-3, Pro: p=5.5e-1). FIG. 2I is a summary of findings: AD sequences (ATF4 (SEQ ID NO: 17445), JADE2 (SEQ ID NO: 17594), NR4A1 (SEQ ID NO: 71674), TET2 (SEQ ID NO: 17798), KLF4 (SEQ ID NO: 17749), BRD4 (SEQ ID NO: 17455), BRD4 (SEQ ID NO: 17454), OCT4 (SEQ ID NO: 17706), which facilitate activity consist of hydrophobic residues that are interspersed with acidic, prolines, serines and/or glutamine residues.

FIGS. 3A-3F show repression domain (RD) sequences contain either sites for SUMOylation, short interaction motifs for recruiting co-repressors, or are structured binding domains for recruiting other repressive proteins. FIG. 3A is a count of RDs (repressive in both pEF and PGK promoter screens) that overlap annotations from UniProt and ELM (Eukaryotic Linear Motifs). Annotations that had at least 6 counts are shown. P-values from a one-sided proportions z-test stating how likely it is to find an annotation (e.g., zinc finger) overlapping an activating tile versus a repressing tile: SUMO p=3.7e-26, zinc finger=2.9e-21, DNA binding domain p=1.1e-22, co-repressor binding p=4.7e-4. FIG. 3B is repression enrichment scores (n=2, dots are the mean, vertical bars the range) for tiles that contain a co-repressor binding motif versus a replacement with Ala (Mutant). TLE-binding: 6 lost all repressive activity upon motif removal. Fraction of non-hit sequences containing motif=0. HP1-binding: 8/13 significantly decreased activity upon motif removal (one-tailed z-test). Fraction of non-hit sequences containing motif=0.002. CtBP-binding: 14/17 significantly decreased activity upon motif removal. Fraction of non-hit sequences containing motif-0.002. FIG. 3C is deletion scan across SP3's RD (SEQ ID NO: 2179). SUMOylation motif is “IKEE” (SEQ ID NO: 28213). Blue rectangle is the WT enrichment score, its height the range of two biologically independent screens. Each horizontal line represents which residues were deleted, dots are the mean, vertical bars the range, and p-values less than 0.05 (one-sided z test compared to WT) are labeled in grey as decrease. FIG. 3D shows the fraction of deletion sequences containing a SUMOylation motif binned according to their effect on activity (blue=no change relative to WT, gray=decreased, one-tailed z test, n=166 total RDs). FIG. 3E is a deletion scan across IKZF5's RD (SEQ ID NO: 2063) (n=2, dots are the mean, bars the range). AlphaFold's predicted secondary structure (prediction from whole protein sequence) shown below: alpha helices in green and beta sheets in orange. FIG. 3F is a summary of RD functional sequence categories (n indicated in Figure). SEQ ID NO: 28205 in (1) and SEQ ID NO: 28206 in (2).

FIGS. 4A-4F show bifunctional activating and repressing domains. Bifunctional tiles were discovered by observing both activation above the hits threshold (vertical dashed line in FIG. 4A) in the minCMV promoter CRTF validation screen (x-axis) and repression above the hits threshold (horizontal dashed line) in the pEF promoter CRTF validation screen (y-axis) (n=2 biological replicates for each point). FIG. 4B is citrine distributions from flow cytometry for individual validations of bifunctional tiles. Untreated cells (gray) and dox-treated cells (colors) (n=2 biological replicates in each condition). Vertical line is the citrine gate used to determine the fraction of cells ON for activation and OFF for repression. FIG. 4C is a tiling plot for ARGFX (n=2, dots are the mean, bars the range). Bifunctional domains are regions where the sequence is both activating at the minCMV promoter and repressing at the pEF promoter. FIG. 4D is deletion scans across ARGFX-161:240 (SEQ ID NO: 280) at minCMV promoter (top), and at pEF promoter (bottom). Yellow and blue rectangles represent WT enrichment scores, its height the range of two biologically independent screens. Each horizontal line represents which residues were deleted, dots are the mean, vertical bars the range. The 3 deletions that caused no activation and no repression across both screens are shown in shading and with a bar above the sequence. FIG. 4E is citrine distributions after recruitment of bifunctional tile ARGFX-161:240 to the PGK promoter (n=2). Left vertical gate as used for measuring the fraction of cells OFF to its left. Right vertical gate was used for measuring the fraction of cells HIGH to its right. The fraction of LOW cells was measured by quantifying the number of cells between the two gates. FIG. 4F is fraction of cells with citrine OFF (navy), LOW (gray), and HIGH (pink) over time after recruitment of ARGFX-161:240 (n=2 biological replicates, average plotted as a line).

FIGS. 5A-5G show CRTF tiling screens' separation purity, reproducibility, and validation. FIG. 5A is a comparison between the set of proteins tiled in Tycko et al (See, Tycko, J. et al. Cell 183, 2020-2035.e16 (2020), incorporated herein by reference in its entirety) and those protein identified herein. FIG. 5B is flow cytometry data showing citrine reporter distributions for the minCMV promoter screen on the day localization was induced with dox (Pre-induction), on the day of magnetic separation (Pre-separation), and after separation (Bound). Overlapping histograms are shown for two separately transduced biological replicates. The average percentage of cells ON is shown to the right of the vertical line showing the citrine level gate. FIG. 5C is citrine reporter distributions for the pEF promoter screen (n=2). FIGS. 5D-5E are biological replicate screen reproducibility (for hits above the threshold: pearson r²=0.78 for minCMV and r²=0.19 for pEF; for all data, including noise under the hit threshold: pearson r²=0.66 for minCMV and r²=0.16 for pEF). FIG. 5F is comparison between average repression enrichment scores of tiles that were screened in the CRTF tiling pEF screen (x-axis) and previous silencer tiling screen (y-axis). Dashed lines are the hits thresholds for each screen. Tiles were identical with a 1 aa register shift (as Silencer library tiles included an initial methionine absent from the CRTF tiling library). Pink dots are tiles that were individually validated in FIG. 5G. FIG. 5G is citrine reporter distributions of individually validated CRTF tiling pEF screen hits that were not identified within the Silencer tiling screen (n=2).

FIG. 6A-6D show CRTF tiling FLAG protein expression screen separation purity, reproducibility, validation, and example of how the data were used. FIG. 6A is Alexa Fluor 647 distributions from anti-FLAG staining of the CRTF tiling library in minCMV promoter reporter cells (n=2). FIG. 6B is biological replicate screen reproducibility (pearson r²=0.49). FIG. 6C is validations of FLAG protein expression screen. Expression levels were measured by Western blot with an anti-FLAG antibody. Anti-histone H3 was used as a loading control for normalization. Lane 1: rTetR-3xFLAG (no tile) theoretical molecular weight of 29 kDa; lanes 2-6: rTetR-3xFLAG-screened P53 deletions, theoretical molecular weight of 39 kDa; lanes 7-9: rTetR-3xFLAG-P53's AD loaded at increasing amounts; lanes 10-14: rTetR-3xFLAG-screened random control. Shift from expected molecular weight of the expressed P53 proteins is likely due to post-translational modifications P53's AD undergoes. Comparison between high-throughput measurements of expression and Western blot protein levels (r²=0.87, n=10 proteins, n=2 blot replicates, dots are the mean, bars the range). FIG. 6D is tiling plot for BCL11A (n=2, dots are the mean, bars the range). Example of a domain that was annotated at position 571-710. This domain had a low expression tile in the middle but the domain was left unsegmented.

FIGS. 7A-7F show CRTF tile hits validation screens' separation purity, reproducibility, and validation. FIG. 7A is flow cytometry data showing citrine reporter distributions for the minCMV promoter screen on the day localization was induced with dox (Pre-induction), on the day of magnetic separation (Pre-separation), and after separation (Bound). Overlapping histograms are shown for 2 biological replicates. The average percentage of cells ON is shown to the right of the vertical line showing the citrine level gate. FIG. 7B is citrine reporter distributions for the pEF promoter validation screen (n=2). FIGS. 7C-7D are biological replicate screen reproducibility. FIG. 7E is comparison between individually recruited measurements and minCMV promoter validation screen measurements (n=2, dots are the mean, bars the range) with logistic model fit plotted as solid line (r²=0.91, n=20). Dashed line is the hits threshold. Note, both screen thresholds are below 0, with several validated screen measurements below 0. FIG. 7F is comparison between individually recruited measurements and pEF promoter validation screen measurements (n=2, dots are the mean, bars the range) with logistic model fit plotted as solid line (r²=0.94, n=19).

FIGS. 8A-8H show validations of CR & TF EDs. FIG. 8A is a comparison between set of proteins screened in Alerasool et al. (See, Alerasool, N., et al., Mol. Cell 82, 393 677-695.e7 (2022)) and CRTF tiles. FIG. 8B is net charge per residue distributions (calculated by CIDER) of activation domains identified by HT-recruit compared to their PADDLE-predicted function (Mann-Whitney p-value=1.4e-15, boxes: median and interquartile range (IQR); whiskers: Q1-1.5*IQR and +Q3). FIG. 8C is CRTF tiling library screened at three different promoters with distinct expression levels. minCMV is a minimal promoter with all cells off. PGK is a low expression, medium strength promoter, and pEF is a high expression, strong promoter. FIG. 8D is flow cytometry data showing citrine reporter distributions for the PGK promoter screen on the day localization was induced with dox (Pre-induction), 5 days later on the day of magnetic separation (Pre-separation), and after separation (Bound). Overlapping histograms are shown for 2 biological replicates. The average percentage of cells ON is shown to the right of the vertical line showing the citrine level gate. FIG. 8E is biological replicate PGK promoter screen reproducibility (for hits above the threshold: pearson r²=0.27 for repression hits; for all data, including noise under the hit threshold: pearson r²=0.11 for all data). Although it is possible to detect activators at the PGK promoter, the dynamic range is very small (ten of the strongest activating tiles at the minCMV promoter (black dots) are very close to the random controls (grey dots)). FIG. 8F is validation screen biological replicate reproducibility of tiles that were hits in both the PGK and pEF promoter screens. FIG. 8G is tiling plots for MEF2C and KLF11 (n=2, dots are the mean, bars the range). PGK repression domains annotated in teal. FIG. 8H is comparison of each repression domain's max tile average repression scores in PGK (x-axis) and pEF promoter screen (y-axis). Dashed lines are the hits thresholds for each screen.

FIGS. 9A-9G show mutant AD screen's separation purity, reproducibility, and validation. FIG. 9A is citrine distributions after 2 days recruitment to minCMV of UniProt-annotated Q-rich ADs with or without an 11 aa acidic sequence from VP64 (n=2). FIG. 9B, top, is deletion scan across P53's AD (SEQ ID NO: 28211): Deletions that caused a complete loss of activation, meaning they are below the experimentally validated activation threshold (dotted line, determined in FIG. 1G for the screen that included these constructs), and deletions that retained some activation (n=2, dots are the mean, bars the range). FIG. 9B, bottom, is individual validations of tiles including 15 aa deletions (deleted sequences shown above each panel—SEQ ID NOs: 28207-28210, left to right). Untreated cells (gray) and dox-treated cells (colors) shown with two biological replicates in each condition. Vertical line is the citrine gate used to determine the fraction of cells ON (written above each distribution). FIG. 9C is flow cytometry data showing citrine reporter distributions for the Mutant AD transcriptional activity screen on the day localization was induced with dox (Pre-induction), on the day of magnetic separation (Pre-separation), and after separation (Bound). Overlapping histograms are shown for 2 separately transduced biological replicates. The average percentage of cells ON is shown to the right of the vertical line showing the citrine level gate. FIG. 9D is biological replicate Mutant AD transcriptional activity screen reproducibility. FIG. 9E is comparison between individually recruited measurements and Mutant AD screen measurements (n=2, dots are the mean, bars the range) with logistic model fit plotted as solid line (r²=0.95, n=23). FIG. 9F is Alexa Fluor 647 distributions from anti-FLAG staining. FIG. 9G is biological replicate Mutant AD protein expression screen reproducibility.

FIGS. 10A-10F are mutant AD screen follow-up. FIG. 10A is a deletion scan across SMARCA4's AD (SEQ ID NO: 532) (n=2, dots are the mean, bars the range). Predicted secondary structure (prediction from whole protein sequence using AlphaFold) shown below, where green regions are alpha helices. Deletions that are significantly different from WT are colored in gray (p<0.05, one-tailed z-test). FIG. 10B is enrichment scores comparing WT versus the W, F, Y, L mutant of DUX4 tile 35 (p-value=3.3e-13, one-tailed z-test, n=2, dots are the mean, bars the range). FIG. 10C is violin plots of average FLAG enrichment scores from 2 biological replicates binned by each sublibrary. Dashed line represents the hit threshold for this screen. P-values computed from Mann-Whitney one-sided U tests. Boxes: median and interquartile range (IQR); whiskers: Q1-1.5*IQR and +Q3. FIG. 10D is correlations between each tile's activation strength in the minCMV validation screen and the count of indicated aa. FIG. 10E is a boxplot of acidic count for each mutant's activation category (Decrease n=33, No change n=18). Mann-Whitney one-sided U test, p-value=2.25e-3. Boxes: median and interquartile range (IQR); whiskers: Q1-1.5*IQR and +Q3. FIG. 10F is a boxplot of average activation enrichment scores with interquartile range shown for tiles that contain a single sequence across each category (Acidic n=9 S, P, Q n=9, Mixed n=64). P-values computed from Mann-Whitney one-sided U tests. Boxes: median and interquartile range (IQR); whiskers: Q1-1.5*IQR and +Q3.

FIGS. 11A-11G are distribution of tile's predicted secondary structure, mutant RD screen's separation purity and reproducibility, and HES family tiling plot examples. FIG. 11A is distributions of activating and repressing tile's fraction of the sequence predicted to be structured from AlphaFold's predictions on the full length protein sequence. p-value=4.1e-8 (Mann Whitney U test, one-sided, boxes: median and interquartile range (IQR); whiskers: Q1-1.5*IQR and +Q3). FIG. 11B is flow cytometry data showing citrine reporter distributions for the Mutant RD transcriptional activity screen on the day localization was induced with dox (Pre-induction), on the day of magnetic separation (Pre-separation), and after separation (Bound). Overlapping histograms are shown for 2 separately transduced biological replicates. The average percentage of cells ON is shown to the right of the vertical line showing the citrine level gate. FIG. 11C is biological replicate Mutant RD transcriptional activity screen reproducibility. FIG. 11D is a comparison between individually recruited measurements and mutant RD screen measurements (n=2, dots are the mean, bars the range) with logistic model fit plotted as solid line (r²=0.91, n=9). There are significantly fewer points for this plot compared to others because unlike the mutant AD screen which included all hits that contained a W, F, Y or L, the mutant RD screen had much fewer hits that overlapped the set of validations since only the strongest tiles within domains or hits that contained co-repressor binding motifs were included in the library design FIG. 11E is Alexa Fluor 647 staining distributions for the Mutant RD FLAG protein expression screen. FIG. 11F is biological replicate Mutant RD protein expression screen reproducibility. FIG. 11G is tiling plots for all 7 HES family members (n=2, dots are the mean, bars the range).

FIGS. 12A-12I are mutant RD screen follow-up. FIG. 12A is repression enrichment scores for a subset of repressing tiles (n indicated in figure) that contain a relatively more flexible CtBP-binding motif (regex shown above), excluding the more refined CtBP-binding motif (regex shown on second line). Mutants have their binding motifs replaced with alanines (p-values computed from one-tailed z-test). FIG. 12B is repression enrichment scores for repressing tiles that contain a flexible SUMO-binding motif (fraction of non-hit sequences containing motif=0.155). (n=2, dots are the mean, bars the range, p-values computed from one-tailed z-test). FIG. 12C is the fraction of AD deletion sequences containing a SUMOylation motif binned according to their effect on activity (yellow=no change on activation relative to WT, gray=decreased activation). 11 total ADs. FIG. 12D is a deletion scan across TCF15's RD (SEQ ID NO: 1947) (n=2, dots are the mean, bars the range). Deletions are colored by whether they were above or below the experimentally validated detection threshold for repression (dotted line). AlphaFold60's predicted secondary structure (prediction from whole protein sequence) shown below where green regions are alpha helices. Annotations shown from protein accession NP_004600.3 FIG. 12E is distribution of bHLH classifications of RDs overlapping bHLH UniProt annotations. Classifications taken from Torres-Machorro, A. L. Int. J. Mol. Sci. 22, (2021), incorporated herein by reference in its entirety. FIG. 12F is a deletion scan across REST's RD (n=2, dots are the mean, bars the range). Deletions are colored by whether they were above or below the validated threshold. AlphaFold's predicted secondary structure (prediction from whole protein sequence) shown below where green regions are alpha helices and orange arrows are beta sheets. FIG. 12G is tiling plots for IKZF family members (n=2, dots are the mean, bars the range. FIG. 12H is deletion scan across IKZF1, 2 and 4's RDs (n=2, dots are the mean, bars the range). Deletions are colored by whether they were above or below the validated threshold. FIG. 12I is a cartoon model of potential mechanisms corresponding to the RD categories in FIG. 3F.

FIGS. 13A-13G are bifunctional domain deletion scan screen's separation purity, reproducibility, and examples. FIG. 13A is counts of bifunctional domains from proteins that contain the indicated DNA binding domains. Homeodomains are enriched among TFs containing bifunctional domains compared to the frequency of homeodomains among all TFs (p=2.5e-4, Fisher's exact test, two-sided). FIG. 13B is a tiling plot for NANOG (n=2, dots are the mean, bars the range). FIG. 13C is flow cytometry data showing citrine reporter distributions for the bifunctional deletion scan minCMV promoter screen on the day localization was induced with dox (Pre-induction), on the day of magnetic separation (Pre-separation), and after separation marker (Bound). Overlapping histograms are shown for 2 separately transduced biological replicates. The average percentage of cells ON is shown to the right of the vertical line showing the citrine level gate. FIG. 13D is a biological replicate bifunctional deletion scan minCMV promoter screen reproducibility. FIG. 13E is citrine reporter distributions for the bifunctional deletion scan pEF promoter screen (n=2). FIG. 13F is biological replicate bifunctional deletion scan pEF promoter screen reproducibility. FIG. 13G is example of a bifunctional domain from NANOG (SEQ ID NO: 238) with independent activating and repressing regions (n=2, dots are the mean, bars the range). Note, deletion of the sequence for activation, caused an increase in repression, and vice-versa.

FIGS. 14A-14F are examples of bifunctional domain sequences at three different promoters. FIG. 14A is a tiling plot for LEUTX (n=2, dots are the mean, bars the range). FIG. 14B is a deletion scan across one of LEUTX's bifunctional tiles (SEQ ID NO: 757) (n=2, dots are the mean, bars the range). Deletions were binned by their statistical significance into those that decreased activity (gray lines) compared to the WT tile and those that did not (one-tailed z-test). The sequence for another gene family member, ARGFX, is highlighted in teal. FIG. 14C is bifunctional domain region location categories. Overlapping regions were defined as any tile that contained a deletion that facilitated activation and repression. FIG. 14D is citrine distributions of ARGFX-161:240 recruited to minCMV (n=2, left), and recruited to pEF (n=2, right). FIG. 14E is citrine distributions of bifunctional tiles identified from minCMV and pEF CRTF tiling screens recruited to PGK promoter (n=2). Asterisks denote p-values<0.05 for the percentage of cells on (right) and off (left) in the dox population (one-sided Welch's t-test, unequal variance). ARGFX-191:270 off p=0.0003, on p=0.02; FOXO1-561:640 off p=0.017, on p=2.44e-5; NANOG 191:270 off p=2.12e-5, on p=0.0002; NANOG 225:304 off p=0.202, on p=0.0004; KLF7 1:80 off p=0.99, on p=0.0005. FIG. 14F is comparison between set of proteins screened in Alerasool et al. (See, Alerasool, N., et al., Mol. Cell 82, 393 677-695.e7 (2022)), and this study.

FIG. 15 is a schematic of high-throughput recruitment (HT-recruit) to quantify transcriptional effector function at scale while varying the context of DNA-binding domains (DBDs), cell type, and target reporters or endogenous genes. A pooled library of tiles is synthesized as 300-mer DNA oligonucleotides, cloned downstream of the doxycycline (dox)-inducible rTetR DNA-binding domain (DBD) or dCas9, and delivered to K562 cells at a low multiplicity of infection (MOI) such that the majority of cells express a single DBD-domain fusion. The target gene (inset) can be silenced or activated by recruitment of repressor or activator domains to the promoter. The synthetic reporters can be driven by different promoters and encode a synthetic surface marker (Igκ-hIgG1-Fc-PDGFRβ, purple) and fluorescent marker (Citrine, yellow), separated by a T2A self-cleaving peptide (gray). These reporters are stably integrated into the AAVS1 safe harbor locus using TALEN-mediated homology directed repair. The endogenous target genes encode for surface markers. After recruitment of Pfam domains, ON and OFF cells were magnetically separated using beads that bind these synthetic or endogenous surface markers (when stained with antibodies), and the domains were sequenced in the Bound and Unbound populations to compute enrichments.

FIG. 16 is a schematic of lentiviruses used for HT-recruit with dCas9 to target endogenous genes. One lentivirus encodes dCas9 and a cloning site for the library of protein sequences, and the second delivers an sgRNA that targets the transcriptional start site of an endogenous gene.

FIG. 17 is graphs of the validation of sgRNAs to silence or repress endogenous surface markers with known effector domains. Expression of endogenous surface marker genes CD2 and CD43 in K562 cells as measured by immunostaining and flow cytometry. dCas9 fusions and sgRNAs were delivered by lentivirus and selected for by blasticidin and puromycin. Data shown after gating for sgRNA delivery (mCherry⁺ in CD43 and GFP⁺ in CD2 samples) and for dCas9 (BFP⁺) (n=1 infection replicate).

FIGS. 18A-18E show dCas9 fusions to tiles of all human chromatin regulator and transcription factors uncovers unannotated effectors. FIG. 18A is a schematic of a library tiling all human chromatin regulator and transcription factor (CR & TF) proteins in 80 amino acid tiles with a 10 amino acid step size (n=128,565 elements) fused to dCas9 and used to target CD43 with sg15 and CD2 with sg717. FIG. 18B shows dCas9 recruitment of CR & TF tiles to CD2 compared with rTetR recruitment to minCMV. Dashed lines show hit threshold at 2 standard deviations below the median of the random controls (n=2 replicates per screen). FIG. 18C shows tiling of SWI/SNF proteins SMARCA4 and SMARCC2, and the PHD protein JADE1. Each horizontal line is a tile, and vertical bars show the range (n=2 screen replicates). Dashed horizontal line is the hit calling threshold based on random controls. UniProt annotations and Pfam domains are shown below. FIG. 18D shows the comparison of dCas9 recruitment to CD43 with rTetR recruitment to pEF1a. Dashed lines show hit threshold at 2 standard deviations above the median of the random controls (n=2 replicates per screen). FIG. 18E is tiling of methyl-binding domain related proteins GATAD2B and MBD3. Each horizontal line is a tile, and vertical bars show the range (n=2 screen replicates). Dashed horizontal line is the hit calling threshold based on random controls.

FIGS. 19A-19E show the CRISPR HT-recruit of library tiling human transcription factors and chromatin regulators. FIG. 19A is replicate correlation of CR & TF library fused to dCas9 and recruited to CD43 or CD2 in K562 cells. Hit threshold shown at 2 standard deviations above (for CD43 screen) or below (CD2) the median of the random controls. FIG. 19B is ranking of tiles and random controls by the sum of their mean repression scores from the pEF and CD43 screens (n=2 replicates per screen). The ZNF705E tile is 99% identical to the ZNF705B/D/F KRAB described earlier, which was not itself included in the library. FIG. 19C is tiling of HLH protein NeuroG2. Each horizontal line is a tile, and vertical bars show the range (n=2 screen replicates). Blue lines show repression of pEF and orange lines show activation of CD2. Dashed horizontal line is the hit calling threshold based on random controls. Red box shows shared hit region for both repression and activation. UniProt annotations and Pfam domains are shown below. FIG. 19D is tiling of HLH protein ASCL4. FIG. 19E is a comparison of dCas9 recruitment to CD2 with rTetR recruitment to pEF1a. Dashed lines show hit threshold at 2 standard deviations below or above the median of the random controls (n=2 replicates per screen). Some example hits are labeled with their protein, and the labels are orange for HLH proteins.

### DETAILED DESCRIPTION

Human gene expression is regulated by over 2,000 transcription factors and chromatin regulators. Effector domains within these proteins can activate or repress transcription. However, for many of these regulators it is unknown what type of effector domains they contain, their location in the protein, their activation and repression strengths, and the sequences that are necessary for their functions. Here, the effector activity of >100,000 protein fragments tiling across most chromatin regulators and transcription factors in human cells (2,047 proteins) was systematically measured. By testing the effect they have when recruited at reporter genes, 374 activation domains and 715 repression domains were identified, ˜80% of which were not previously known. Rational mutagenesis and deletion scans across the effector domains revealed aromatic and/or leucine residues interspersed with acidic, proline, serine, and/or glutamine residues facilitate activation domain activity. Additionally, most repression domain sequences contained either sites for SUMOylation, short interaction motifs for recruiting co-repressors, or structured binding domains for recruiting other repressive proteins. Surprisingly, bifunctional domains were discovered that can both activate and repress, some of which dynamically split a cell population into high- and low-expression subpopulations.

The provided catalog of effector domains, which when fused onto DNA binding domains, can be used to engineer synthetic transcription factors. These find use to perform targeted and tunable regulation of gene expression in cells (e.g., eukaryotic cells). A high-throughput platform was used to screen and characterize tens of thousands of synthetic transcription factors in cells. These synthetic transcription factors are fusions between a DNA binding domain and a transcriptional effector domain. The targeting of these fusions generates local regulation of mRNA transcription, either negatively or positively depending on the effector domain. Some of these synthetic transcription factors mediate long-term epigenetic regulation that persists after the factor itself has been released from the target.

Previously, a limited number of transcriptional effector domains were available for the engineering of synthetic transcription factors. A high-throughput approach was used to screen and quantify the function of transcriptional effectors domains, identifying domains that can upregulate or downregulate transcription in a targeted manner when fused onto a DNA binding domain. This process also finds use to identify mutants of effector domains with enhanced activity. These effector domains find use to engineer synthetic transcription factors for applications in gene and cell therapy, synthetic biology, and functional genomics.

Exemplary applications include, but are not limited to: targeted repression/activation of endogenous genes with fusions of programmable DNA binding domains (e.g., dCas9, dCas12a, zinc finger, TALE) to transcriptional effector domains; gene and cell therapy (e.g., to silence a pathogenic transcript in a patient) or in research; perturbation of the expression of multiple genes simultaneously (e.g., to perform high-throughput genetic interaction mapping with CRISPRi/a screens using multiple guide RNAs) and use as synthetic transcription factors in genetic circuits, e.g., inducible gene expression or more complex circuits, which find use in gene therapy (e.g., AAV delivery of antibodies) and cell therapy (e.g., ex vivo engineering of CAR-T cells) to achieve therapeutic gene expression outputs in response to environmental and small molecule inputs.

The new transcriptional effector domains provided herein have several advantages for applications that rely on synthetic transcription factors. In some embodiments, the domains are extracted from human proteins, which provides the advantage of reducing immunogenicity in comparison to viral effector domains. Most of the domains generated have not been reported as transcriptional effectors previously. In addition, a high-throughput process may be used for testing mutations in these domains in order to identify enhanced variants.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, cell and tissue culture, molecular biology, immunology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

“Heterologous” as used herein, refers to a macromolecules and compounds (e.g., nucleic acids, proteins, polypeptides, etc.) which originate from a foreign source (or species) or, if from the same source, is modified from its original form. As such, when used in the context of a nucleic acid or polypeptide heterologous refers to a nucleic acid or protein that is not normally found in a given cell in nature. The term encompasses a nucleic acid or polypeptide wherein at least one of the following is true: (a) a nucleic acid or polypeptide that is exogenously introduced into a given cell; (b) the nucleic acid or polypeptide is recombinant or was produced by synthetic means; and (c) the nucleic acid or polypeptide may comprise sequences, segments, domains, or other portions that are not found in the same relationship to each other in nature.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

As used herein, “treat,” “treating,” and the like means a slowing, stopping, or reversing of progression of a disease or disorder. The term also means a reversing of the progression of such a disease or disorder to a point of eliminating or greatly reducing the symptoms. As such, “treating” means an application or administration of the compositions or conjugates described herein to a subject, where the subject has a disease or a symptom of a disease, where the purpose is to cure, heal, alleviate, relieve, alter, remedy, ameliorate, improve, or affect the disease or symptoms of the disease.

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (e.g., altered characteristics) when compared to the wild-type gene or gene product. It is noted that naturally occurring mutants can be isolated; these are identified by the fact that they have altered characteristics when compared to the wild-type gene or gene product.

### 2. TRANSCRIPTION FACTORS

The present disclosure provides synthetic transcription factors comprising one or more transcriptional effector domains fused to a heterologous DNA binding domain. As used herein, the term “transcription factor” refers to a protein or polypeptide that interacts with, directly or indirectly, specific DNA sequences associated with a genomic locus or gene of interest to block or recruit RNA polymerase activity to the promoter site for a gene or set of genes.

In some embodiments the synthetic transcription factor comprises one or more activator domains, one or more repressor domains, or a combination thereof fused to a heterologous DNA binding domain. In some embodiments, the one or more activator domains or the one or more repressor domains comprises an amino acid sequence having at least 70% (e.g., at least 75%, at least 80%, at least 85%, at least 90%, at least 95%, at least 98%, at least 99%) identity to any of SEQ ID NOS: 1-12567 and 28214-28404. In some embodiments, the one or more activator domains or the one or more repressor domains comprises SEQ ID NOS: 1-12567 and 28214-28404. In some embodiments, the one or more activator domains or the one or more repressor domains comprises an amino acid sequence comprising at least 10, at least 20, at least 30, at least 40, at least 50, at least 60, or at least 70 contiguous amino acids of any one of SEQ ID NOS: 1-12567 and 28214-28404.

In some embodiments, the one or more activator domains comprises an amino acid sequence having at least 70% (e.g., at least 75%, at least 80%, at least 85%, at least 90%, at least 95%, at least 98%, at least 99%) identity to any of SEQ ID NOs: 31, 36, 111, 113, 153, 158, 165, 182, 184, 189, 224, 291, 311, 313, 352, 362, 367, 369, 375, 381, 407, 410, 415, 426, 430, 436, 472, 476, 478, 480, 483, 487-489, 494, 496, 498, 509, 512-517, 524, 526, 527, 530, 532, 533, 537, 541, 542, 545-547, 549, 552, 554, 557, 560-562, 565-568, 570-576, 578, 579, 580, 581, 582, 585, 587, 589, 590, 592, 595-598, 601, 603, 605, 607, 613, 617, 620, 622-624, 626, 627, 629, 630, 634-636, 639, 643, 646, 648, 651, 654, 658, 659, 662, 664, 666, 673, 675, 677, 678, 681, 684, 685, 686, 687, 689, 695, 696, 697, 699, 704, 705, 707-711, 713, 715, 716, 721, 723-725, 728, 729, 731-733, 735, 744, 746, 747, 753, 755, 760, 761, 764, 766-769, 773, and 775-984. In some embodiments, the one or more activator domains comprises SEQ ID NOs: 31, 36, 111, 113, 153, 158, 165, 182, 184, 189, 224, 291, 311, 313, 352, 362, 367, 369, 375, 381, 407, 410, 415, 426, 430, 436, 472, 476, 478, 480, 483, 487-489, 494, 496, 498, 509, 512-517, 524, 526, 527, 530, 532, 533, 537, 541, 542, 545-547, 549, 552, 554, 557, 560-562, 565-568, 570-576, 578, 579, 580, 581, 582, 585, 587, 589, 590, 592, 595-598, 601, 603, 605, 607, 613, 617, 620, 622-624, 626, 627, 629, 630, 634-636, 639, 643, 646, 648, 651, 654, 658, 659, 662, 664, 666, 673, 675, 677, 678, 681, 684, 685, 686, 687, 689, 695, 696, 697, 699, 704, 705, 707-711, 713, 715, 716, 721, 723-725, 728, 729, 731-733, 735, 744, 746, 747, 753, 755, 760, 761, 764, 766-769, 773, and 775-984. In some embodiments, the one or more activator domains comprises an amino acid sequence comprising at least 10, at least 20, at least 30, at least 40, at least 50, at least 60, or at least 70contiguous amino acids of any one of SEQ ID NOs: 31, 36, 111, 113, 153, 158, 165, 182, 184, 189, 224, 291, 311, 313, 352, 362, 367, 369, 375, 381, 407, 410, 415, 426, 430, 436, 472, 476, 478, 480, 483, 487-489, 494, 496, 498, 509, 512-517, 524, 526, 527, 530, 532, 533, 537, 541, 542, 545-547, 549, 552, 554, 557, 560-562, 565-568, 570-576, 578, 579, 580, 581, 582, 585, 587, 589, 590, 592, 595-598, 601, 603, 605, 607, 613, 617, 620, 622-624, 626, 627, 629, 630, 634-636, 639, 643, 646, 648, 651, 654, 658, 659, 662, 664, 666, 673, 675, 677, 678, 681, 684, 685, 686, 687, 689, 695, 696, 697, 699, 704, 705, 707-711, 713, 715, 716, 721, 723-725, 728, 729, 731-733, 735, 744, 746, 747, 753, 755, 760, 761, 764, 766-769, 773, and 775-984.

In some embodiments, at least one of the one or more activator domains comprises an amino acid sequence having at least 70% identity to any of SEQ ID NOs: 88, 144, 147, 148, 149, 234, 280 281, 282, 283, 302, 306, 307, 322, 355, 356, 440, 441, 442, 443, 444, 445, 446, 447, 448, 449, 477, 488, 501, 532, 548, 593, 610, 618, 676, 738, 757, and 28365-28404.

In some embodiments, the one or more activator domains comprises an amino acid sequence having at least 70% (e.g., at least 75%, at least 80%, at least 85%, at least 90%, at least 95%, at least 98%, at least 99%) identity to any of SEQ ID NOs: 12568-13273. In some embodiments, the one or more activator domains comprises SEQ ID NOs: 12568-13273.

In some embodiments, the one or more activator domains comprises an amino acid sequence having at least 70% (e.g., at least 75%, at least 80%, at least 85%, at least 90%, at least 95%, at least 98%, at least 99%) identity to any of SEQ ID NOs: 13274-17423. In some embodiments, the one or more activator domains comprises SEQ ID NOs: 13274-17423.

In some embodiments, the one or more activator domains comprises one or more of SEQ ID NOs: 17424-17841.

In some embodiments, the one or more repressor domains comprises an amino acid sequence having at least 70% (e.g., at least 75%, at least 80%, at least 85%, at least 90%, at least 95%, at least 98%, at least 99%) identity to any of SEQ ID NOs: 1036, 1054, 1055, 1069, 1120, 1144, 1182, 1183, 1200, 1208, 1314, 1318, 1366, 1402, 1417, 1442, 1516, 1518, 1543, 1598, 1627, 1655, 1665, 1667, 1670, 1706, 1710, 1711, 1735, 1738, 1742, 1747, 1748, 1752, 1756, 1763, 1777, 1783, 1786, 1789, 1793, 1794, 1808, 1811, 1822, 1831, 1838, 1839, 1854, 1859, 1862, 1865, 1866, 1869, 1870, 1872, 1875, 1883, 1889, 1891, 1893, 1901, 1902, 1905, 1907, 1910, 1912, 1913, 1914, 1915, 1916, 1922, 1923, 1927, 1930, 1934, 1940, 1944, 1946, 1948, 1951, 1952, 1956, 1957, 1968, 1969, 1972, 1987, 1992, 1994, 1996, 2004, 2007, 2010, 2017, 2022, 2029, 2033, 2041, 2042, 2043, 2048, 2050, 2051, 2053, 2057, 2064, 2095, 2107, 2112, 2119, 2123, 2128, 2131, 2139, 2150, 2157, 2160, 2163, 2176, 2182, 2188, 2190, 2192, 2193, 2194, 2205, 2206, 2207, 2208, 2211, 2212, 2213, 2216, 2218, 2221, 2224, 2227, 2231, 2232, 2239, 2245, 2246, 2254, 2262, 2263, 2265, 2271, 2274, 2275, 2277, 2278, 2282, 2283, 2288, 2292, 2295, 2296, 2298, 2302, 2312, 2313, 2316, 2320, 2321, 2323, 2324, 2325, 2334, 2338, 2341, 2348, 2361, 2364, 2365, and 2370-6094. In some embodiments, the one or more repressor domains comprises SEQ ID NOs: 1036, 1054, 1055, 1069, 1120, 1144, 1182, 1183, 1200, 1208, 1314, 1318, 1366, 1402, 1417, 1442, 1516, 1518, 1543, 1598, 1627, 1655, 1665, 1667, 1670, 1706, 1710, 1711, 1735, 1738, 1742, 1747, 1748, 1752, 1756, 1763, 1777, 1783, 1786, 1789, 1793, 1794, 1808, 1811, 1822, 1831, 1838, 1839, 1854, 1859, 1862, 1865, 1866, 1869, 1870, 1872, 1875, 1883, 1889, 1891, 1893, 1901, 1902, 1905, 1907, 1910, 1912, 1913, 1914, 1915, 1916, 1922, 1923, 1927, 1930, 1934, 1940, 1944, 1946, 1948, 1951, 1952, 1956, 1957, 1968, 1969, 1972, 1987, 1992, 1994, 1996, 2004, 2007, 2010, 2017, 2022, 2029, 2033, 2041, 2042, 2043, 2048, 2050, 2051, 2053, 2057, 2064, 2095, 2107, 2112, 2119, 2123, 2128, 2131, 2139, 2150, 2157, 2160, 2163, 2176, 2182, 2188, 2190, 2192, 2193, 2194, 2205, 2206, 2207, 2208, 2211, 2212, 2213, 2216, 2218, 2221, 2224, 2227, 2231, 2232, 2239, 2245, 2246, 2254, 2262, 2263, 2265, 2271, 2274, 2275, 2277, 2278, 2282, 2283, 2288, 2292, 2295, 2296, 2298, 2302, 2312, 2313, 2316, 2320, 2321, 2323, 2324, 2325, 2334, 2338, 2341, 2348, 2361, 2364, 2365, and 2370-6094. In some embodiments, the one or more repressor domains comprises an amino acid sequence comprising at least 10, at least 20, at least 30, at least 40, at least 50, at least 60, or at least 70 contiguous amino acids of any one of SEQ ID NOs: 1036, 1054, 1055, 1069, 1120, 1144, 1182, 1183, 1200, 1208, 1314, 1318, 1366, 1402, 1417, 1442, 1516, 1518, 1543, 1598, 1627, 1655, 1665, 1667, 1670, 1706, 1710, 1711, 1735, 1738, 1742, 1747, 1748, 1752, 1756, 1763, 1777, 1783, 1786, 1789, 1793, 1794, 1808, 1811, 1822, 1831, 1838, 1839, 1854, 1859, 1862, 1865, 1866, 1869, 1870, 1872, 1875, 1883, 1889, 1891, 1893, 1901, 1902, 1905, 1907, 1910, 1912, 1913, 1914, 1915, 1916, 1922, 1923, 1927, 1930, 1934, 1940, 1944, 1946, 1948, 1951, 1952, 1956, 1957, 1968, 1969, 1972, 1987, 1992, 1994, 1996, 2004, 2007, 2010, 2017, 2022, 2029, 2033, 2041, 2042, 2043, 2048, 2050, 2051, 2053, 2057, 2064, 2095, 2107, 2112, 2119, 2123, 2128, 2131, 2139, 2150, 2157, 2160, 2163, 2176, 2182, 2188, 2190, 2192, 2193, 2194, 2205, 2206, 2207, 2208, 2211, 2212, 2213, 2216, 2218, 2221, 2224, 2227, 2231, 2232, 2239, 2245, 2246, 2254, 2262, 2263, 2265, 2271, 2274, 2275, 2277, 2278, 2282, 2283, 2288, 2292, 2295, 2296, 2298, 2302, 2312, 2313, 2316, 2320, 2321, 2323, 2324, 2325, 2334, 2338, 2341, 2348, 2361, 2364, 2365, and 2370-6094.

In some embodiments, the one or more repressor domains comprises an amino acid sequence having at least 70% (e.g., at least 75%, at least 80%, at least 85%, at least 90%, at least 95%, at least 98%, at least 99%) identity to any of SEQ ID NOs: 17842-24889. In some embodiments, the one or more repressor domains comprises SEQ ID NOs: 17842-24889.

In some embodiments, the one or more repressor domains comprises one or more of SEQ ID NOs: 24890-25651.

In some embodiments, the one or more activator domains or the one or more repressor domains comprises an amino acid sequence having at least 70% (e.g., at least 75%, at least 80%, at least 85%, at least 90%, at least 95%, at least 98%, at least 99%) identity to any of the sequences found in SEQ ID NOs: 25652-28198. In some embodiments, the one or more activator domains or the one or more repressor domains comprises SEQ ID NOs: 25652-28198.

In some embodiments, the synthetic transcription factor comprises two or more transcription effector domains (e.g., activator domains, repressor domains, or a combination thereof) fused to a heterologous DNA binding domain. In some embodiments, the synthetic transcription factor comprises two or more activator domains or two or more repressors domains fused to a heterologous DNA binding domain. The two or more effector domains can be fused to the DNA binding domain in any orientation, and may be separated from each other with an amino acid linker. In select embodiments, the synthetic transcription factor comprises two or more transcription effector domains (e.g., activator domains, repressor domains, or a combination thereof) fused to a heterologous DNA binding domain.

In some embodiments, when the synthetic transcription factor comprises more than one transcription effector domains, the synthetic transcription factor may comprise at least one activator domain or at least one repressor domain as disclosed herein with at least one additional effector domain known in the art. See for example, Tycko J. et al., Cell. 2020 Dec 23;183(7):2020-2035, incorporated herein by reference in its entirety. In some embodiments, the one or more activator domain, the one or more repressor domain is identified by the methods described herein.

In some embodiments, when the synthetic transcription factor comprises more than one transcription effector domains, at least one of the one or more transcriptional effector domains comprising an effector domain as disclosed above and herein. For example, in some embodiments, at least one of the one or more transcriptional effector domains comprises an amino acid sequence having at least 70% identity to any of SEQ ID NOS: 1-12567 and 28214-28404.

The DNA binding domain is any polypeptide which is capable of binding double- or single-stranded DNA, generally or with sequence specificity. DNA binding domains include those polypeptides having helix-turn-helix motifs, zinc fingers, leucine zippers, HMG-box (high mobility group box) domains, winged helix region, winged helix-turn-helix region, helix-loop-helix region, immunoglobulin fold, B3 domain, Wor3 domain, TAL effector DNA-binding domain and the like. The heterologous DNA binding domains may be a natural binding domain. In some embodiments, the heterologous DNA binding domain comprises a programmable DNA binding domain, e.g., a DNA binding domain engineered, for example by altering one or more amino acids of a natural DNA binding domain to bind to a predetermined nucleotide sequence.

In some embodiments, the DNA binding domain is capable of binding directly to the target DNA sequences.

The DNA-binding domain may be derived from domains found in naturally occurring Transcription activator-like effectors (TALEs), such as AvrBs3, Hax2, Hax3 or Hax4 (Bonas et al. 1989. Mol Gen Genet 218(1): 127-36; Kay et al. 2005 Mol Plant Microbe Interact 18(8): 838-48). TALEs have a modular DNA-binding domain consisting of repetitive sequences of residues; each repeat region consists of 34 amino acids. A pair of residues at the 12th and 13th position of each repeat region determines the nucleotide specificity and combining of the regions allows synthesis of sequence-specific TALE DNA-binding domains. In some embodiments, the TALE DNA binding domains may be engineered using known methods to provide a DNA binding domain with chosen specificity for any target sequence. The DNA binding domain may comprise multiple (e.g., 2, 3, 4, 5, 6, 10, 20, or more) Tal effector DNA-binding motifs. In particular, any number of nucleotide-specific Tal effector motifs can be combined to form a sequence-specific DNA-binding domain to be employed in the present transcription factor.

In some embodiments, the DNA binding domain associates with the target DNA in concert with an exogenous factor.

In some embodiments, the DNA binding domain is derived from a Clustered Regularly Interspaced Short Palindromic Repeats associated (Cas) protein (e.g., catalytically dead Cas9) and associates with the target DNA through a guide RNA. The gRNA itself comprises a sequence complementary to one strand of the DNA target sequence and a scaffold sequence which binds and recruits Cas9 to the target DNA sequence. The transcription factors described herein may be useful for CRISPR interference (CRISPRi) or CRISPR activation (CRISPRa).

The guide RNA (gRNA) may be a crRNA, crRNA/tracrRNA (or single guide RNA, sgRNA). The gRNA may be a non-naturally occurring gRNA. The terms “gRNA,” “guide RNA” and “guide sequence” may be used interchangeably throughout and refer to a nucleic acid comprising a sequence that determines the binding specificity of the Cas protein. A gRNA hybridizes to (complementary to, partially or completely) the DNA target sequence.

The gRNA or portion thereof that hybridizes to the target nucleic acid (a target site) may be any length necessary for selective hybridization. gRNAs or sgRNA(s) can be between about 5 and about 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLOS ONE, 10(3): (2015)); Zhu et al. (PLOS ONE, 9(9) (2014)); Xiao et al. (Bioinformatics. Jan 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

The present disclosure also provides synthetic transcription factors comprising one or more transcriptional effector domains fused to an exogenous factor which associates with a second exogenous factor comprising a DNA binding domain. Such inducible systems include, but not limited to, tetracycline Tet,/DOX inducible systems, light inducible systems, Abscisic acid (ABA) inducible systems, cumate systems, 40HT/estrogen inducible systems, ecdysone-based inducible systems, and FKBP12/FRAP (FKBP12-rapamycin complex) inducible systems.

The transcription effector domain(s) and the DNA binding domain(s) may be fused in any orientation. In some embodiments, the transcription effector domain(s) are N-terminal to the DNA binding domain(s). In some embodiments, the transcription effector domain(s) are C-terminal to the DNA binding domain(s). For example, in some embodiments, the N-terminus of the transcription effector domain(s) are fused to the C-terminus of the DNA binding domain(s). In some embodiments, the C-terminus of the transcription effector domain(s) are fused to the N-terminus of the DNA binding domain(s). In some embodiments, the N-terminus of the transcription effector domain(s) are fused to the N-terminus of the DNA binding domain(s). In some embodiments, the C-terminus of the transcription effector domain(s) are fused to the C-terminus of the DNA binding domain(s).

The transcription effector domain(s) and the DNA binding domain(s) may be fused via a linker polypeptide. The linker polypeptide may have any of a variety of amino acid sequences. Proteins can be joined by a spacer peptide, generally of a flexible nature, although other chemical linkages are not excluded. Suitable linkers include polypeptides of between 4 amino acids and 100 amino acids in length. These linkers can be produced by using synthetic, linker-encoding oligonucleotides to couple the transcription effector domain(s) and the DNA binding domain(s), or can be encoded by a nucleic acid sequence encoding the transcription factors.

In some embodiments, the linker peptides are flexible linkers. The linking peptides may have virtually any amino acid sequence, with preferred linkers having a sequence that results in a generally flexible peptide. A variety of different linkers are suitable for use, including but not limited to, glycine-serine polymers, glycine-alanine polymers, and alanine-serine polymers. In some embodiments, the linker comprises at least one glycine and at least one serine. In some embodiments, the linker comprises an amino acid sequence consisting of (Gly2Ser) n, where n is the number of repeats comprising an integer from 2-20.

In some embodiments, the transcription factors comprise a nuclear localization sequence (NLS). The nuclear localization sequence may be appended, for example, to the N-terminus, a C-terminus, or a combination thereof of the transcription factor. The transcription factor may comprise two or more NLSs. The two or more NLSs may be in tandem, separated by a linker, at either end terminus of the transcription factor, or one or more may be embedded in the transcription factor (e.g., between the transcription effector domain(s) and the DNA binding domain(s)).

The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine. The NLS may be appended to the nuclease by a linker. The linker may be a polypeptide of any amino acid sequence and length.

In some embodiments, the NLS is a monopartite sequence. A monopartite NLS comprise a single cluster of positively charged or basic amino acids. In some embodiments, the monopartite NLS comprises a sequence of K-K/R-X-K/R, wherein X can be any amino acid. Exemplary monopartite NLS sequences include those from the SV40 large T-antigen, c-Myc, and TUS-proteins. In some embodiments, the NLS is a bipartite sequence. Bipartite NLSs comprise two clusters of basic amino acids, separated by a spacer of about 9-12 amino acids. Exemplary bipartite NLSs include the nuclear localization sequences of nucleoplasmin, EGL-12, or bipartite SV40.

The transcription factors may comprise an epitope tag (e.g., 3xFLAG tag, an HA tag, a Myc tag, and the like). The epitope tags may be at the N-terminus, a C-terminus, or a combination thereof of the transcription factors. In some embodiments, the epitope tag may be adjacent, either upstream or downstream, to a nuclear localization sequence.

The transcription factors may comprise another protein or protein domain. For example, the transcription factors may be fused to another protein or protein domain that provides for tagging or visualization (e.g., GFP). The transcription factors may be fused to a protein or protein domain that has another functionality or activity useful to target to certain DNA sequences (e.g., nuclease activity such as that provide by FokI nuclease, protein modification activity such as histone modification activity including acetylation or deacetylation or demethylation or methyltransferase activity, base editing activity such as deaminase activity, DNA modifying activity such as DNA methylation activity, and the like).

In some embodiments, the transcription factors may be fused with one or more (e.g., two, three, four, or more) protein transduction domains or PTDs, also known as a CPP-cell penetrating peptide. A protein transduction domains is a polypeptide, polynucleotide, carbohydrate, or organic or inorganic compound that facilitates traversing a lipid bilayer, micelle, cell membrane, organelle membrane, or vesicle membrane. A PTD attached to another molecule, facilitates the molecule traversing a membrane, for example going from extracellular space to intracellular space, or cytosol to within an organelle. In some embodiments, a PTD is covalently linked to a terminus of the transcription factor (e.g., N-terminus, C-terminus, or both). In some embodiments, the PTD is inserted internally at a suitable insertion site. Examples of PTDs include but are not limited to a minimal undecapeptide protein transduction domain (corresponding to residues 47-57 of HIV-1 TAT comprising); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9 (6): 489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); Transportan, and the like.

The present disclosure also provides nucleic acids encoding a synthetic transcription factor or a transcriptional effector (e.g., activator or repressor) domain, as disclosed herein. In some embodiments, the nucleic acid encodes one or more synthetic transcription factor or one or more effector domain.

Nucleic acids of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible expression can be accomplished by placing the nucleic acid encoding such a molecule under the control of an inducible promoter/regulatory sequence. Promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The present disclosure also provides for vectors containing the nucleic acids and cells containing the nucleic acids or vectors, thereof. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

To construct cells that express the present transcription factors, expression vectors for stable or transient expression of the present system may be constructed via conventional methods and introduced into cells. For example, nucleic acids encoding the components the disclose transcription factors, or other nucleic acids or proteins, may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2,cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

The vectors of the present disclosure may direct the expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene for selection of stable or transient transfectants in host cells; transcription termination and RNA processing signals; 5′- and 3′-untranslated regions; internal ribosome binding sites (IRESes), versatile multiple cloning sites; and reporter gene for assessing expression of the chimeric receptor. Suitable vectors and methods for producing vectors containing transgenes are well known and available in the art. Selectable markers include chloramphenicol resistance, tetracycline resistance, spectinomycin resistance, neomycin, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into a cell, the vectors may be maintained as an autonomously replicating sequence or extrachromosomal element or may be integrated into host DNA.

Thus, the disclosure further provides for cells comprising a synthetic transcription factor, a nucleic acid, or a vector, as disclosed herein.

Conventional viral and non-viral based gene transfer methods can be used to introduce the nucleic acids into cells, tissues, or a subject. Such methods can be used to administer the nucleic acids to cells in culture, or in a host organism. Non-viral vector delivery systems include DNA plasmids, cosmids, RNA (e.g., a transcript of a vector described herein), a nucleic acid, and a nucleic acid complexed with a delivery vehicle.

Viral vector delivery systems include DNA and RNA viruses, which have either episomal or integrated genomes after delivery to the cell. A variety of viral constructs may be used to deliver the present nucleic acids to the cells, tissues and/or a subject. Viral vectors include, for example, retroviral, lentiviral, adenoviral, adeno-associated and herpes simplex viral vectors. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2):249-71, incorporated herein by reference.

The nucleic acids or transcription factors may be delivered by any suitable means. In certain embodiments, the nucleic acids or proteins thereof are delivered in vivo. In other embodiments, the nucleic acids or proteins thereof are delivered to isolated/cultured cells in vitro or ex vivo to provide modified cells useful for in vivo delivery to patients afflicted with a disease or condition.

Vectors according to the present disclosure can be transformed, transfected, or otherwise introduced into a wide variety of host cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan 1;459(1-2):70-83), incorporated herein by reference.

As such, the disclosure provides an isolated cell comprising the vector(s) or nucleic acid(s) disclosed herein. Preferred cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993), incorporated herein by reference. Desirably, the cell is a mammalian cell, and in some embodiments, the cell is a human cell. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or HaK hamster cell lines.

Methods for selecting suitable mammalian cells and methods for transformation, culture, amplification, screening, and purification of cells are known in the art.

The present invention is also directed to compositions or systems comprising a synthetic transcription factor, a nucleic acid, a vector, or a cell, as described herein. In some embodiments, the compositions or system comprises two or more synthetic transcription factors, nucleic acids, vectors, or cells.

In some embodiments, the composition or system further comprises a gRNA. The gRNA may be encoded on the same nucleic acid as a synthetic transcription factor or a different nucleic acid. In some embodiments, the vector encoding a synthetic transcription factor may further encode a gRNA, under the same or different promoter. In some embodiments, the gRNA is encoded on its own vector, separated from that of the transcription factor.

### 3. METHODS OF MODULATING GENE EXPRESSION

The present disclosure also provides methods of modulating the expression of at least one target gene in a cell, the method comprising introducing into the cell one or more of the effector domains, at least one synthetic transcription factor, nucleic acid, vector, or composition or system as described herein. In some embodiments, the gene expression of at least two genes is modulated.

In some embodiments, the gene is an endogenous gene. In some embodiments, the gene is an exogenous gene. In some embodiments, the gene is on an exogenous vector. In some embodiments, the exogenous gene was introduced into the cell as part of a gene therapy regime. For example, a controllable and activatable vector expressing secreted hepatocyte growth factor has broad therapeutic potential due to its capacity to induce regeneration of health tissues when transduced into the tissue or interest or neighboring tissues (e.g., liver to regenerate damaged liver or kidney, heart for prevention of/and regeneration after heart attack, brain for neurogenesis in Alzheimer's and Parkinson's diseases).

Modulation of expression comprises increasing or decreasing gene expression compared to normal gene expression for the target gene. When the gene expression of at least two genes is modulation, both genes may have increased gene expression, both gene may have decreased gene expression, or one gene may have increased gene expression and the other may have decreased gene expression. To determine the level of gene expression modulation by a transcriptional effector or transcription factor, cells contacted with a transcriptional effector or transcription factor are compared to control cells, e.g., without the transcriptional effector or transcription factor, to examine the extent of inhibition or activation based on a measured value for gene expression (e.g., transcript levels or gene product (e.g., protein levels)).

In some embodiments, expression of the gene is reduced by about 10% (e.g., 90% of control expression), about 50% (e.g., 50% of control expression), about 20% (e.g., 80% of control expression), about 50% (e.g., 50% of control expression), or about 75-100% (e.g., 25% to 0% of control expression). In some embodiments, expression is increased by about 10% (e.g., 110% of control expression), about 20% (e.g., 120% of control expression), about 50% (e.g., 150% of control expression), about 100% (e.g., 200% of control expression), about 5-10 fold (e.g., 500-1000% of control expression), up to at least 100 fold or more.

The cell may be a prokaryotic or eukaryotic cell. In select embodiments, the cell is a eukaryotic cell. In certain embodiments, the cell is a human cell. In some embodiments, the cell is in vitro. In some embodiments, the cell is ex vivo.

In some embodiments, the cell is in an organism or host, such that introducing the disclosed systems, compositions, vectors into the cell comprises administration to a subject. The method may comprise providing or administering to the subject, in vivo, or by transplantation of ex vivo treated cells, at least one synthetic transcription factor, nucleic acid, vector, or composition or system as described herein.

A “subject” may be human or non-human and may include, for example, animal strains or species used as “model systems” for research purposes, such a mouse model, prokaryotic models (e.g., bacteria), archea, and single-celled eukaryotes (e.g., yeast). Likewise, subject may include either adults or juveniles (e.g., children). Moreover, subject may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

As used herein, the terms “providing”, “administering,” “introducing,” are used interchangeably herein and refer to the placement of the transcription factors of the disclosure, or nucleic acids encoding the transcription factors, into a subject by a method or route which results in at least partial localization to a desired site. The transcription factors of the disclosure, or nucleic acids encoding the transcription factors, can be administered by any appropriate route which results in delivery to a desired location in the subject.

The transcription factors, or nucleic acids encoding the transcription factors, may be administered to a cell or subject with a pharmaceutically acceptable carrier or excipient as a pharmaceutical composition. In some embodiments, the c transcription factors of the disclosure, or nucleic acids encoding the transcription factors, may be mixed, individually or in any combination, with a pharmaceutically acceptable carrier to form pharmaceutical compositions, which are also within the scope of the present disclosure.

The phrase “pharmaceutically acceptable,” refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the transcription factors of the disclosure, or nucleic acids encoding the transcription factors, and does not negatively affect the subject to which the composition(s) are administered. Any of the pharmaceutical compositions used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The route by which the transcription factors of the disclosure, or nucleic acids encoding the transcription factors, are administered and the form of the composition will dictate the type of carrier to be used. The transcription factors of the disclosure, or nucleic acids encoding the transcription factors, may be administered systemically or topically, and therefore, the composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis).

The methods described herein for modulating gene expression allow for therapeutic applications, e.g., treatment of genetic diseases; cancer; fungal, protozoal, bacterial, and viral infections; ischemia; vascular disease; arthritis; immunological disorders; etc., as well as providing components for functional genomics assays, and methods for developing plants with altered phenotypes, including disease resistance, fruit ripening, sugar and oil composition, yield, and color.

In some embodiments, the gene is known to be associated with a disease or disorder. In some embodiments, the methods disclosed herein alleviate a symptom associated with the disease or disorder. Thus, the methods, transcription factors, and/or nucleic acids encoding the transcription factors disclosed herein may be used for therapeutic or prophylactic purposes.

The transcription factors, by nature of their DNA binding domains, can be designed to recognize any suitable target site, for regulation of expression of any endogenous gene of choice. Suitable genes to be regulated include, but are not limited to: cytokines, lymphokines, growth factors, mitogenic factors, chemotactic factors, onco-active factors, receptors, potassium channels, G-proteins, signal transduction molecules, and other disease-related genes. Examples of endogenous genes suitable for regulation include, but are not limited to: VEGF, CCR5, ERa, Her2/Neu, Tat, Rev, HBV C, S, X, and P, LDL-R, PEPCK, CYP7, Fibrinogen, ApoB, Apo E, Apo(a), renin, NF-κB, I-κB, TNF-α, FAS ligand, amyloid precursor protein, atrial naturetic factor, ob-leptin, ucp-1, IL-I, IL-2, IL-3, IL-4, IL-5, IL-6, IL-12, G-CSF, GM-CSF, Epo, PDGF, PAF, p53, Rb, fetal hemoglobin, dystrophin, eutrophin, GDNF, NGF, IGF-I, VEGF receptors fit and flk, topoisomerase, telomerase, bcl-2, cyclins, angiostatin, IGF, ICAM-I, STATS, c-myc, c-myb, TH, PTI-I, polygalacturonase, EPSP synthase, FAD2-1, delta-12 desaturase, delta-9 desaturase, delta-15 desaturase, acetyl-CoA carboxylase, acyl-ACP-thioesterase, ADP-glucose pyrophosphorylase, starch synthase, cellulose synthase, sucrose synthase, senescence-associated genes, heavy metal chelators, fatty acid hydroperoxide lyase, viral genes, protozoal genes, fungal genes, and bacterial genes.

In some embodiments, the transcription factors and resulting methods target a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1(1):192 (2008); Online Mendelian Inheritance in Man (OMIM); and the Human Gene Mutation Database (HGMD). Diseases caused by the contribution of multiple genes which lack simple (e.g., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects. In another embodiment, the transcription factors and resulting methods target a cancer oncogene.

The amount of the transcription factors required for use in the disclosed methods will vary not only with the effector domains selected but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages can be determined by comparing their in vitro activity, and in vivo activity in animal models.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

Regulation of gene expression in plants with transcriptional effectors can be used to engineer plants for traits such as increased disease resistance, modification of structural and storage polysaccharides, flavors, proteins, and fatty acids, fruit ripening, yield, color, nutritional characteristics, improved storage capability, and the like. In particular, the engineering of crop species for enhanced oil production, e.g., the modification of the fatty acids produced in oilseeds, is of interest. Thus, the methods, transcription factors, and/or nucleic acids encoding the transcription factors disclosed herein may be used for overall gene regulation in plants and for genetic engineering in plants.

### 4. KITS

Also within the scope of the present disclosure are kits including at least one or all of at least one nucleic acid encoding an effector domain, or a DNA binding domain, or a combination thereof, at least one synthetic transcription factor, or nucleic acid encoding thereof, vectors encoding at least one effector domain or at least one synthetic transcription factor, a composition or system as described herein, a cell comprising an effector domain, a DNA binding domain, a synthetic transcription factor, or a nucleic acid encoding any of thereof, a reporter cell as described herein and a two-part reporter gene as described herein or a nucleic acid encoding thereof.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may include instructions for use in any of the methods described herein. The instructions can comprise a description of use of the components for the methods of identifying repressor domains or methods of modulating gene expression.

The kits provided herein are in suitable packaging. Suitable packaging includes, but is not limited to, vials, bottles, jars, flexible packaging, and the like.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system or composition. The device may include an infusion device, an intravenous solution bag, a hypodermic needle, a vial, and/or a syringe.

### 5. EXAMPLES

### Methods

Cell culture All experiments presented here were carried out in K562 cells (ATCC, CCL-243, female). Cells were cultured in a controlled humidified incubator at 37 C and 5% CO₂, in RPMI 1640 (Gibco, 11-875-119) media supplemented with 10% FBS (Takara, 632180), and 1% Penicillin Streptomycin (Gibco, 15-140-122). HEK293T-LentiX (Takara Bio, 632180, female) cells, used to produce lentivirus, as described below, were grown in DMEM (Gibco, 10569069) media supplemented with 10% FBS (Takara, 632180) and 1% Penicillin Streptomycin Glutamine (Gibco, 10378016). pEF and minCMV promoter reporter cell lines were generated by TALEN-mediated homology-directed repair to integrate donor constructs (pEF promoter: Addgene #161927, minCMV promoter: Addgene #161928) into the AAVS1 locus by electroporation of K562 cells with 1000 ng of reporter donor plasmid and 500 ng of each TALEN-L (Addgene #35431) and TALEN-R (Addgene #35432) plasmid (targeting upstream and downstream the intended DNA cleavage site, respectively). After 7 days, the cells were treated with 1000 ng/mL puromycin antibiotic for 5 days to select for a population where the donor was stably integrated in the intended locus. Fluorescent reporter expression was measured by microscopy and by flow cytometry. The PGK reporter cell line was generated by electroporation of K562 cells with 0.5 ug each of plasmids encoding the AAVS1 TALENs and 1 ug of donor reporter plasmid using program T-016 on the Nucleofector 2b (Lonza, AAB-1001). Cells were treated with 0.5 ug/mL puromycin for one week to enrich for successful integrants. The PGK reporter donor plasmid generated in this study is available from Addgene (Addgene #196545). These cell lines were not authenticated. All cell lines tested negative for mycoplasma.

TF tiling library design 1,294 human transcription factors (TFs) were selected from Lambert, S. A. et al. Cell 175, 598-599 (2018). To make this library's size feasible for high throughput measurements, 476 proteins previously characterized with HT-recruit (See, Tycko, J. et al. Cell 183, 2020-2035.e16 (2020), incorporated herein by reference in its entirety) were excluded: a set of 132 CRs and 344 KRAB-containing TFs. The canonical transcript of each gene was retrieved from Ensembl and chosen using the APPRIS principle transcript. If no APPRIS tag was found, the transcript was chosen using the TSL principle transcript. If no TSL tag was found, the longest transcript with a protein coding CDS was retrieved. The coding sequences were divided into 80 aa tiles with a 10 aa sliding window. For each gene, a final tile was included spanning from 80 aa upstream of the last residue to that last residue, such that the C-terminal region would be included in the library. Duplicate sequences were removed, sequences were codon matched for human codon usage, 7xC homopolymers were removed, BsmBI restriction sites were removed, rare codons (less than 10% frequency) were avoided, and the GC content was constrained to be between 20% and 75% in every 50 nucleotide window (performed with DNA chisel). To improve the coverage of this large library, it was subdivided into 3 smaller sub-libraries based on the three major classes of TFs: a 25,032 C2H2 ZF sub-library including all 406 C2H2 ZF TFs, a 9,757 Homeodomain and bHLH sub-library including all 304 Homeodomain and bHLH TFs, and a 31,664 member sub-library containing the rest of the 583 TFs.

One thousand random controls of 80 aa lacking stop codons were computationally generated as controls using the DNA chisel package's random_dna_sequence function and included in each sub-library. Four hundred seventy-three sequences that were found to be non-activators and forty-two sequences that were found to be activators in a previous minCMV Nuclear Pfam screen were included as negative and positive controls. Alternative codon usage (match_codon_usage, and use_best_codon functions) was used to re-code the controls in each sub-library to give the option of pooling the 3 sub-libraries and running the library as one 73,288 element screen.

One hundred additional controls were added to each sub-library to serve as fiduciary markers to aid comparing separately run screens. These controls were not recoded in each sub-library, and thus were repeated when pooling sub-libraries.

Fifty activation domains from forty-five proteins involved in transcriptional activation were curated from UniProt3. The UniProt database was queried for human proteins whose regions, motifs or annotations included the term “transcriptional activation” and then filtered for ADs that ranged in length from 30 to 95 aa. For ADs shorter than 95 aa, the protein sequence was extended equally on either side until it reached 95 aa. The protein sequences were reverse translated and further divided into 95 aa sequences with 15 aa deletions positioned with a 2 aa sliding window. Duplicate sequences were removed, sequences were codon matched for human codon usage, 7xC homopolymers were removed, BsmBI restriction sites were removed, rare codons (less than 10% frequency) were avoided, and the GC content was constrained to be between 20% and 75% in every 50 nucleotide window, performed with DNA chisel. Fifty yeast Gen4 controls were added, which included previously studied deletions. Two-thousand twenty-four library elements in total were added to the 31,664 element TF tiling sub-library.

CR tiling library design Candidate genes were initially chosen by including all members of the EpiFactors database, genes with gene name prefixes that matched any genes in the EpiFactors database, and genes with any of the following GO terms: GO:000785 (chromatin), GO:0035561 (regulation of chromatin binding), GO:0016569 (covalent chromatin modification), GO:1902275 (regulation of chromatin organization), GO:0003682 (chromatin binding), GO:0042393 (histone binding), GO:0016570 (histone modification), and GO:0006304 (DNA modification). Genes present in prior silencer tiling screens and genes present in the TF tiling screen were then filtered out. Biomart was used to identify and retrieve the canonical transcript, and chosen by (in order of priority) the APPRIS principal transcript, the TSL principal transcript, or the longest transcript with a protein coding CDS. Tiles for each of these DNA sequences were generated using the same 80 aa tile/10 aa sliding window approach as the TF tiling library. Duplicate sequences were removed, DNA hairpins and 7xC homopolymers were removed, and sequences were codon matched for human codon usage with GC content being constrained to be between 20% and 75% globally and between 25% and 65% in any 50-bp window. In order to improve the coverage while performing the screen, this 51,297 element library was split into two sub-libraries: a 38,241 element CR Tiling Main sub-library and an 13,056 element CR Tiling Extended sub-library. Computationally generated random negative controls, negative control tiles from the DMD protein screened in prior Nuclear Pfam screens, and fiduciary marker controls were added to each sub-library: 1,700 elements to the Main sub-library and 3,700 elements to the Extended sub-library. These controls were not re-coded, and thus were repeated when pooling sub-libraries.

Library filtering Since the sub-libraries were pooled and screened as one large pool, several of the control sub-libraries, that were not re-coded, wound up being repeated in the pool several times. Sequences that were repeated upwards of five times had systematically lower enrichment scores than what was expected from previous screens, likely due to PCR bias. Therefore, all repeated control elements were removed and individual validations were instead relied on to confirm screens. Additionally, there was a computational error in removing BsmBI sites from the CR tiling library, resulting in some sequences having accidental restriction cut sites in the middle of the ORF. These sequences were removed from further analysis and supplementary tables.

Activating hits validation library design One thousand fifty-five putative hit tiles were chosen by selecting all tiles where both biological replicates were recovered and had activation enrichment scores above 5.365 (determined by 2 standard deviations above the mean of poorly expressed random controls). Two hundred randomly selected random negative controls that were poorly expressed (expression threshold=−1.427) and one hundred randomly selected non-hit tiles that had no activity in both the minCMV and the pEF CRTF tiling screens were included. There were 1,355 total library elements.

Repressing hits validation library design Nine-thousand, four hundred and thirty-eight putative hit tiles were chosen by selecting all tiles where both biological replicates were recovered and had pEF repression enrichment scores above 1.433 or had a PGK repression enrichment score above 0.880 (determined from 3 standard deviations above the mean of poorly expressed random controls). Five hundred randomly selected random negative controls that were poorly expressed (expression threshold=−1.427) and one hundred randomly selected non-hit tiles that had no activity in the minCMV, pEF nor PGK CRTF tiling screens were included. There were 10,038 total library elements.

AD mutants library design A compositional bias was defined as any residue that represented more than 15% of the sequence (more than 12 residues). Four hundred twenty-four compositionally biased tiles were replaced with alanine. One thousand fifty-five aromatic or leucine-containing tiles replaced all Ws, Fs, Ys, and Ls with alanine. One thousand fifty-two acidic residue-containing tiles replaced all Ds and Es with alanine. Fifty-one tiles that contained the “LxxLL” motif (ELM accession: ELME000045, regex pattern=[\hat{ }P]L[\hat{ }P][\hat{ }P]LL[\hat{ }P]) were replaced with alanine. Twenty-two tiles that contained the “WW” motif (ELM accession: ELME000003, regex pattern=PP.Y) were replaced with alanine. 8,205 deletions were designed by systematically removing 10 aa chunks, with a sliding window of 5 aa from 547 max activating tiles. All mutated sequences were reverse translated into DNA sequences using a probabilistic codon optimization algorithm, such that each DNA sequence contains some variation beyond the substituted residues, which improves the ability to unambiguously align sequencing reads to unique library members. The 1,055 putative hit tiles were included as positive controls. Five hundred randomly selected random negative controls that were poorly expressed (expression threshold=−1.427) were included. There were 12,364 total library elements.

RD mutants library design Twelve thousand deletions were designed by systematically removing 10 aa chunks, with a sliding window of 5 aa of the maximum tile from 800 putative RDs that were hits in both PGK and pEF CRTF tiling screens. All mutated sequences were reverse translated into DNA using the method described above. The 1,593 putative hit tiles were included as positive controls. Six hundred forty-four compositionally biased tiles replaced all residues with alanine. All the following motifs were replaced with alanines: 104 CtBP interaction motif containing tiles (ELM accession: ELME0000098); 18 HP1 interaction motif containing tiles (ELM accession: ELME000141); 9 “ARKS” motif containing tiles (ELM accession: DRAFT-LIG_CHROMO); 180 SUMO interaction motif containing tiles (ELM accession: ELME000335); and 7 WRPW motif containing tiles (ELM accession: ELME000104). Five hundred randomly selected random negative controls that were poorly expressed (expression threshold=−1.427) were included. There were 15,055 total library elements.

Bifunctional deletion scan library design Three thousand three hundred thirty-one deletions were created by systematically removing 10 aa chunks, with a sliding window of 2 aa from 96 bifunctional activating and repressing tiles. All mutated sequences were reverse translated into DNA sequences using the method described above. The WT bifunctional tiles and 250 randomly selected random negative controls that were poorly expressed (expression threshold=−1.427) were included. There were 3,674 total library elements.

Library cloning Oligonucleotides with lengths up to 300 nucleotides were synthesized as pooled libraries (Twist Biosciences) and then PCR amplified. Reactions (6×50 ul) were set up in a clean PCR hood to avoid amplifying contaminating DNA. Each reaction used either 5 or 10 ng of template, 1 ul of each 10 mM primer, 1 ul of Herculase II polymerase (Agilent), 1 ul of DMSO, 1 ul of 10 mM dNTPs, and 10 ul of 5×Herculase buffer. The thermocycling protocol was 3 minutes at 98 C, then cycles of 98 C for 20 s, 61 C for 20 s, 72 C for 30 s, and then a final step of 72 C for 3 minutes. The default cycle number was 20×, and this was optimized for each library to find the lowest cycle that resulted in a clean visible product for gel extraction (in practice, 23 cycles was the maximum when small libraries were represented in large pools). After PCR, the resulting dsDNA libraries were gel extracted by loading a 2% TAE gel, excising the band at the expected length (around 300 bp), and using a QIAgen gel extraction kit. The libraries were cloned into a lentiviral recruitment vector pJT126 (Addgene #161926) with 4-16×10 ul Golden-Gate reactions (75 ng of pre-digested and gel-extracted backbone plasmid, 5 ng of library (2:1 molar ratio of insert:backbone), 2 uL of 10×T4 Ligase Buffer, and 1 uL of NEB Golden Gate Assembly Kit (BsmBI-V2)) with 65 cycles of digestion at 42 C and ligation at 16 C for 5 minutes each, followed by a final 5 minute digestion at 42 C and then 20 minutes of heat inactivation at 70 C. The reactions were then pooled and purified with MinElute columns (QIAgen), eluting in 6 ul of ddH₂O; 2 ul per tube was transformed into two tubes of 50 ml of Endura electrocompetent cells (Lucigen, Cat #60242-2) following the manufacturer's instructions. After recovery, the cells were plated on 1-8 large 10″×10″ LB plates with carbenicillin. After overnight growth in a warm room, the bacterial colonies were scraped into a collection bottle and plasmid pools were extracted with a Hi-Speed Plasmid Maxiprep kit (QIAgen). 2-3 small plates were prepared in parallel with diluted transformed cells in order to count colonies and confirm the transformation efficiency was sufficient to maintain at least 20×library coverage. To determine the quality of the libraries, the putative EDs were amplified from the plasmid pool by PCR with primers with extensions that include Illumina adapters and sequenced. The PCR and sequencing protocols were the same as described below for sequencing from genomic DNA, except these PCRs use 10 ng of input DNA and 17 cycles. These sequencing datasets were analyzed as described below to determine the uniformity of coverage and synthesis quality of the libraries. In addition, 20-30 colonies from the transformations were Sanger sequenced (Quintara) to estimate the cloning efficiency and the proportion of empty backbone plasmids in the pools.

Pooled delivery of library in human cells using lentivirus Large scale lentivirus production and spinfection of K562 cells were performed as follows: To generate sufficient lentivirus to infect the libraries into K562 cells, HEK293T cells were plated on 1-12 15-cm tissue culture plates. On each plate, 8.8×10⁶ HEK293T cells were plated in 30 mL of DMEM, grown overnight, and then transfected with 8 ug of an equimolar mixture of the three third-generation packaging plasmids (pMD2.G, psPAX2, pMDLg/pRRE) and 8 ug of rTetR-domain library vectors using 50 mL of polyethylenimine (PEI, Polysciences #23966). pMD2.G (Addgene plasmid #12259; addgene.org/12259), psPAX2 (Addgene plasmid #12260; addgene.org/12260), and pMDLg/pRRE (Addgene plasmid #12251; addgene.org/12251) were gifts from Didier Trono. After 48 hours and 72 hours of incubation, lentivirus was harvested. The pooled lentivirus was filtered through a 0.45-mm PVDF filter (Millipore) to remove any cellular debris. K562 reporter cells were infected with the lentiviral library by spinfection for 2 hours, with two separate biological replicates infected. Infected cells grew for 2 days and then the cells were selected with blasticidin (10 mg/mL, Gibco). Infection and selection efficiency were monitored each day using flow cytometry to measure mCherry (Biorad ZE5). Cells were maintained in spinner flasks in log growth conditions each day by diluting cell concentrations back to a 5×10⁵ cells/mL. Because lentiviral particles integrate randomly across accessible regions of the genome, the aim was for 600×infection coverage, and the lowest infection coverage was 130× (e.g., 130 cells per library element during infection). The aim was to have 2-10,000×maintenance coverage (e.g., 2-10,000 cells per library element post-infection). On day 8 post-infection, recruitment was induced by treating the cells with 1000 ng/ml doxycycline (Fisher Scientific) for either 2 days for activation or 5 days for repression.

Magnetic separation At each time point, cells were spun down at 300×g for 5 minutes and media was aspirated. Cells were then resuspended in the same volume of PBS (GIBCO) and the spin down and aspiration was repeated, to wash the cells and remove any IgG from serum. Dynabeads M-280 Protein G (ThermoFisher, 10003D) were resuspended by vortexing for 30 s. 50 mL of blocking buffer was prepared per 2×10⁸ cells by adding 1 g of biotin-free BSA (Sigma Aldrich) and 200 mL of 0.5 M pH 8.0 EDTA into DPBS (GIBCO), vacuum filtering with a 0.22-mm filter (Millipore), and then kept on ice. For all activation screens, 30 uL of beads was prepared for every 1×10⁷ cells, 60 uL of beads/10 million cells for the pEF CRTF tiling, PGK CRTF tiling, and minCMV bifunctional deletion scan screens, 120 uL of beads/10 million cells for the pEF validation, 90 uL of beads/10 million cells for the RD Mutants and pEF bifunctional deletion scan screens. Magnetic separation was performed as previously described (See, Tycko, J. et al. Cell 183, 2020-2035.e16 (2020), incorporated herein by reference in its entirety).

FLAG staining for protein expression The expression level measurements for the CRTF tiling library were made in K562 minCMV cells (with citrine OFF). 4×10⁸ cells per biological replicate were used after 7 days of blasticidin selection (10 mg/mL, Gibco), which was 9 days post-infection. 4×10⁷ control K562-JT039 cells (citrine ON, no lentiviral infection) were spiked into each replicate. Fix Buffer I (BD Biosciences, BDB557870) was preheated to 37 C for 15 minutes and Permeabilization Buffer III (BD Biosciences, BDB558050) and PBS (GIBCO) with 10% FBS (Omega) were chilled on ice. The library of cells expressing domains was collected and cell density was counted by flow cytometry (Biorad ZE5). To fix, cells were resuspended in a volume of Fix Buffer I (BD Biosciences, BDB557870) corresponding to pellet volume, with 20 mL per 1 million cells, at 37C for 10-15 minutes. Cells were washed with 1 mL of cold PBS containing 10% FBS, spun down at 500 3 g for 5 minutes and then supernatant was aspirated. Cells were permeabilized for 30 minutes on ice using cold BD Permeabilization Buffer III (BD Biosciences, BDB558050), with 20 mL per 1 million cells, which was added slowly and mixed by vortexing. Cells were then washed twice in 1 mL PBS+10% FBS, as before, and then supernatant was aspirated. Antibody staining was performed for 1 hour at room temperature, protected from light, using 5 uL/1×10⁶ cells of a-FLAG-Alexa647 (RNDsystems, IC8529R). The cells were washed and resuspended at a concentration of 3×10⁷ cells/ml in PBS+10% FBS. Cells were sorted into two bins based on the level of APC-A and mCherry fluorescence (Sony SH800S) after gating for viable cells. A small number of unstained control cells was also analyzed on the sorter to confirm staining was above background. The spike-in citrine positive cells were used to measure the background level of staining in cells known to lack the 3XFLAG tag, and the gate for sorting was drawn above that level. After sorting, the cellular coverage was ˜2000×. The sorted cells were spun down at 500×g for 5 minutes and then resuspended in PBS. Genomic DNA extraction was performed following the manufacturer's instructions (QIAgen Blood Midi kit was used for samples with >1×10⁷ cells) with one modification: the Proteinase K+AL buffer incubation was performed overnight at 56 C.

Library preparation and sequencing Genomic DNA was extracted with the QIAgen Blood Maxi Kit following the manufacturer's instructions with up to 1×10⁸ cells per column. DNA was eluted in EB and not AE to avoid subsequent PCR inhibition. The domain sequences were amplified by PCR with primers containing Illumina adapters as extensions. A test PCR was performed using 5 ug of genomic DNA in a 50 mL (half-size) reaction to verify if the PCR conditions would result in a visible band at the expected size for each sample. Then, 3-48×100 uL reactions were set up on ice (in a clean PCR hood to avoid amplifying contaminating DNA), with the number of reactions depending on the amount of genomic DNA available in each experiment. 10 ug of genomic DNA, 0.5 mL of each 100 mM primer, and 50 mL of NEBnext Ultra 2×Master Mix (NEB) was used in each reaction. The thermocycling protocol was to preheat the thermocycler to 98 C, then add samples for 3 minutes at 98 C, then an optimized number of cycles of 98 C for 10 s, 63 C for 30 s, 72 C for 30 s, and then a final step of 72 C for 2 minutes. All subsequent steps were performed outside the PCR hood. The PCR reactions were pooled and 145 uL were run on a 2% TAE gel, the library band around 395 bp was cut out, and DNA was purified using the QIAquick Gel Extraction kit (QIAgen) with a 30 ul elution into non-stick tubes (Ambion). A confirmatory gel was run to verify that small products were removed. These libraries were then quantified with a Qubit HS kit (Thermo Fisher) and sequenced on an Illumina HiSeq (2×150).

Computing enrichments and hits thresholds Sequencing reads were demultiplexed using bcl2fastq (Illumina). A Bowtie reference (version 1.2.3) was generated using the designed library sequences with the script ‘makeIndices.py’ (HT-Recruit Analyze package) and reads were aligned with 0 mismatch allowance using the script ‘makeCounts.py’. The enrichments for each domain between OFF and ON (or FLAGhigh and FLAGlow) samples were computed using the script ‘makeRhos.py’. Domains with <5 reads in both samples for a given replicate were dropped from that replicate (assigned 0 counts), whereas domains with <5 reads in one sample would have those reads adjusted to 5 in order to avoid the inflation of enrichment values from low depth.

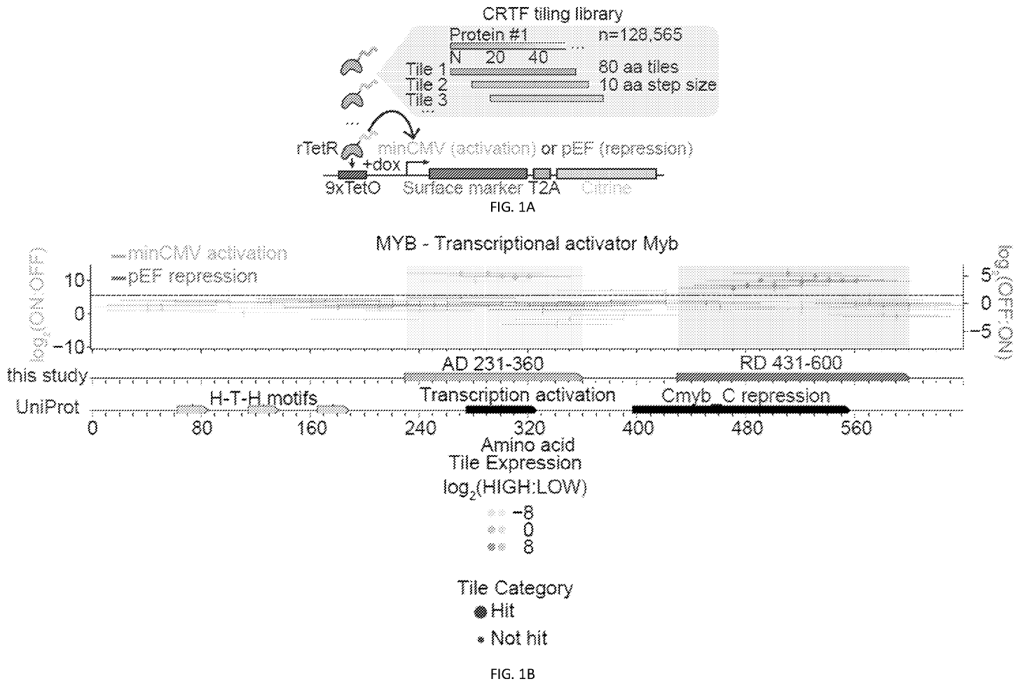

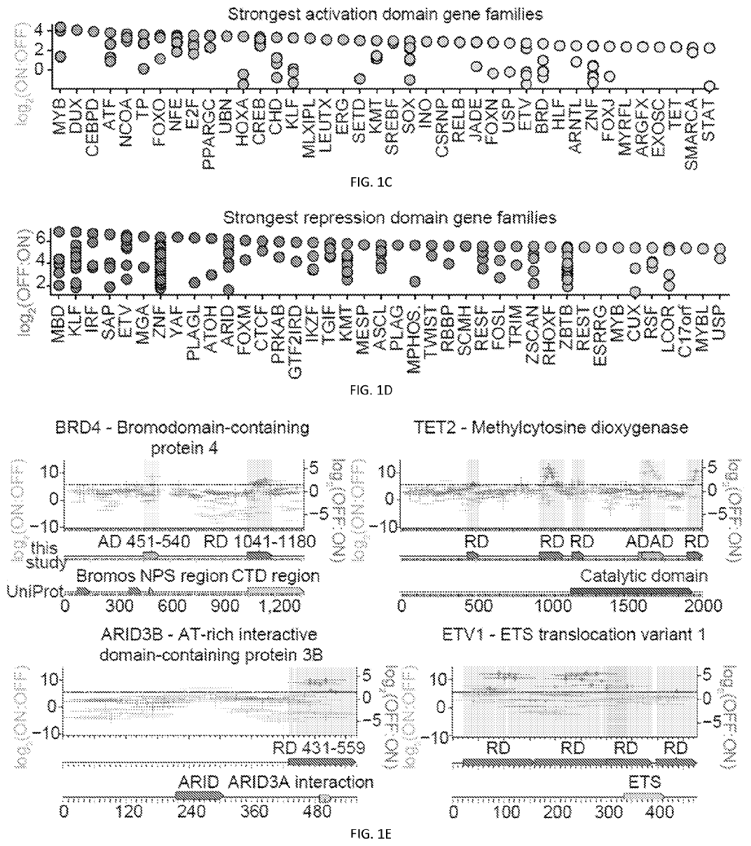

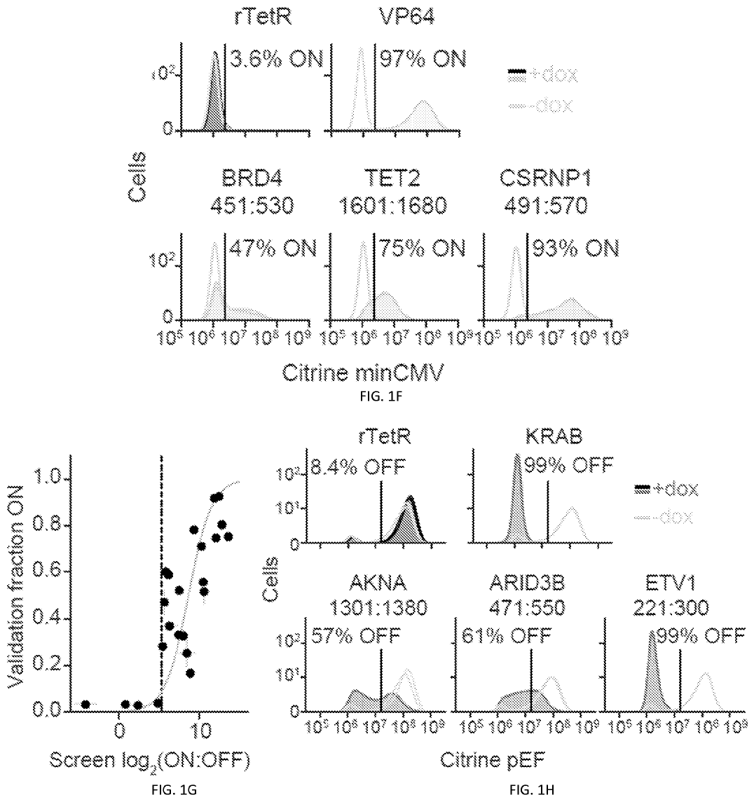

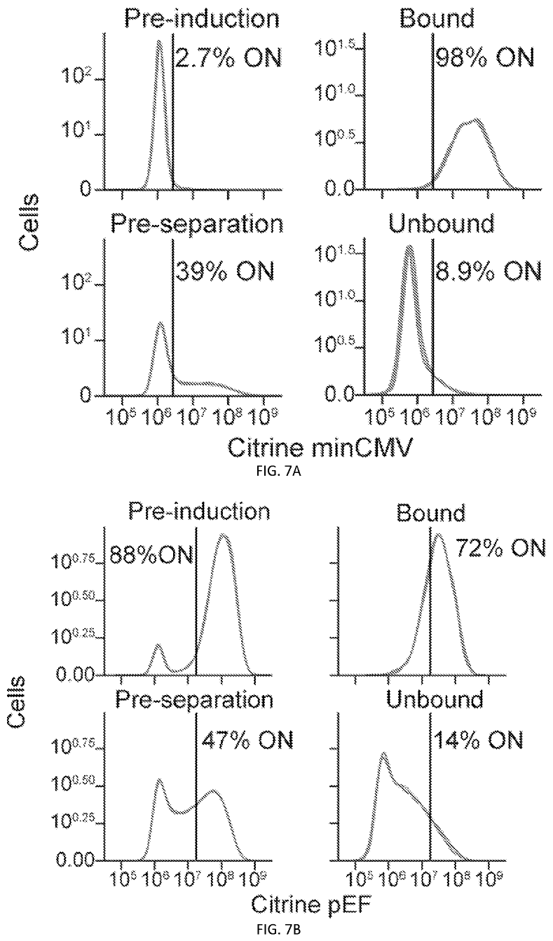

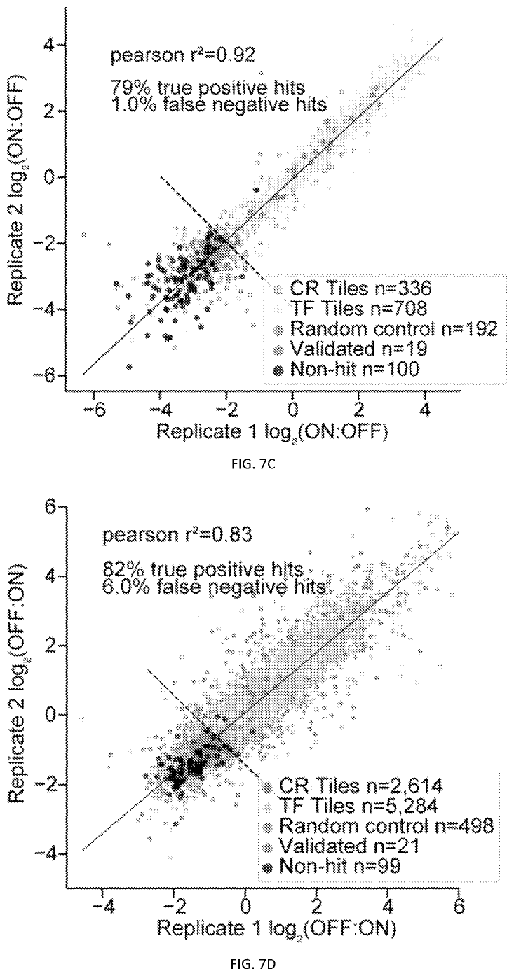

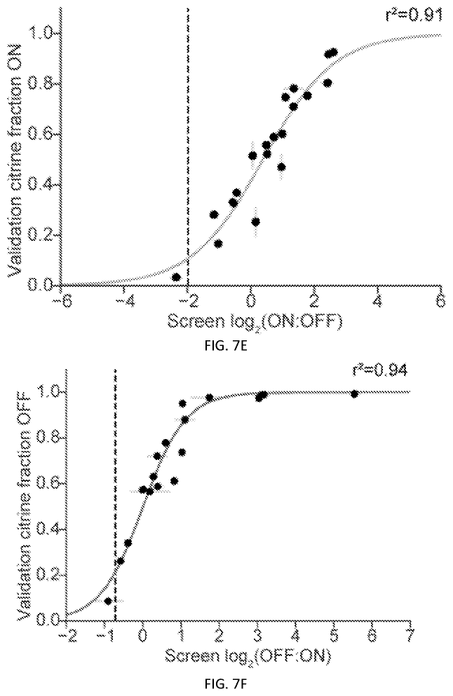

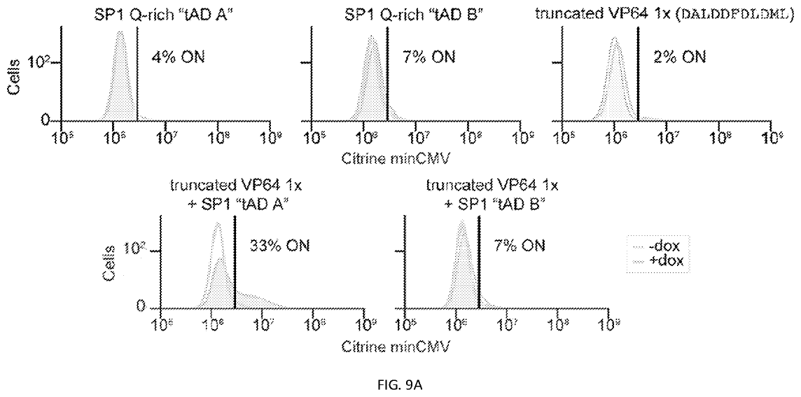

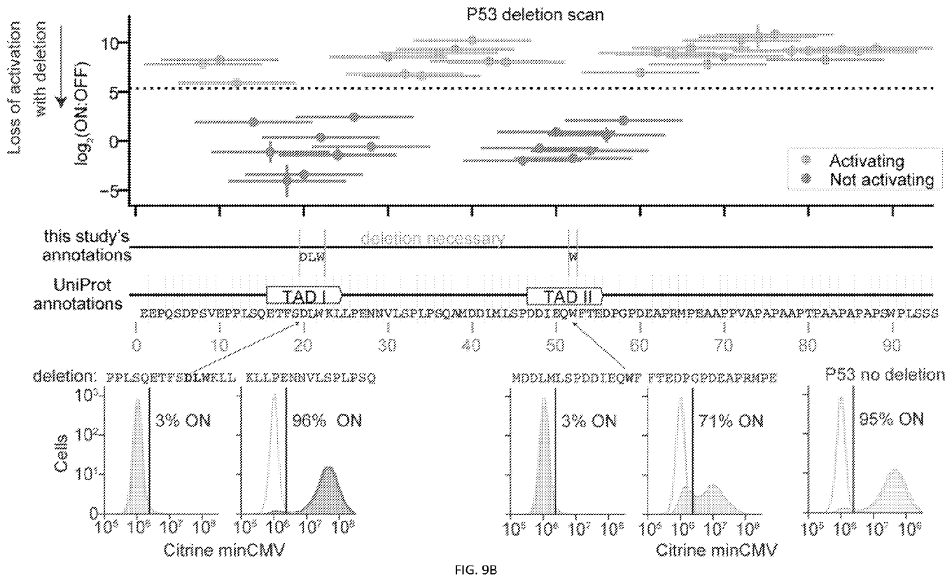

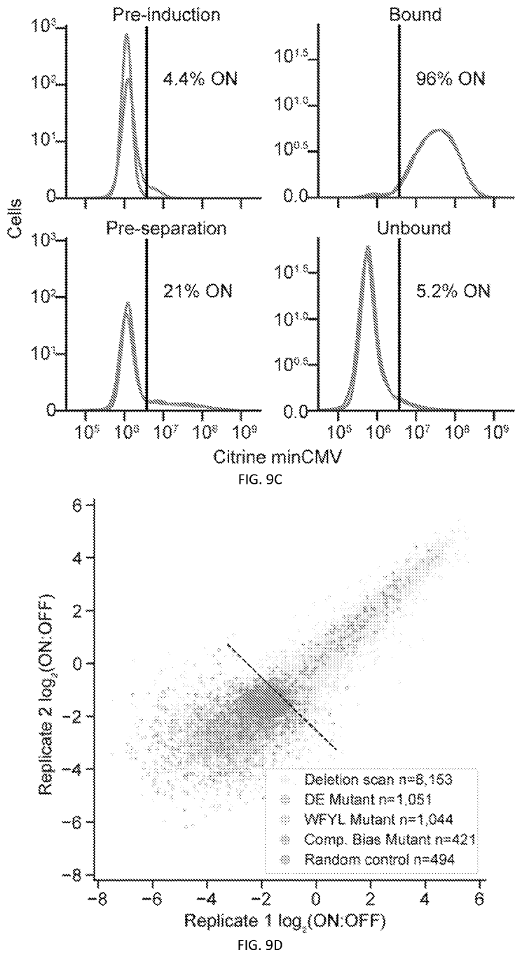

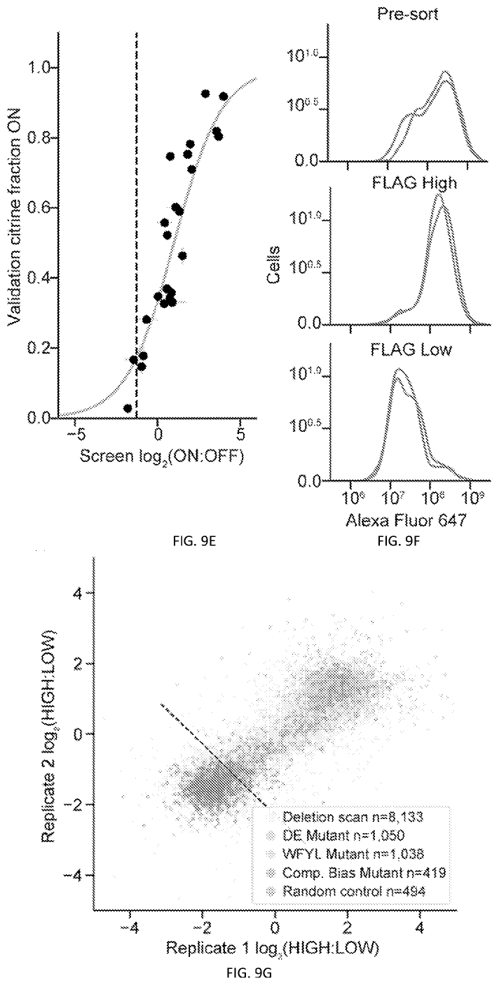

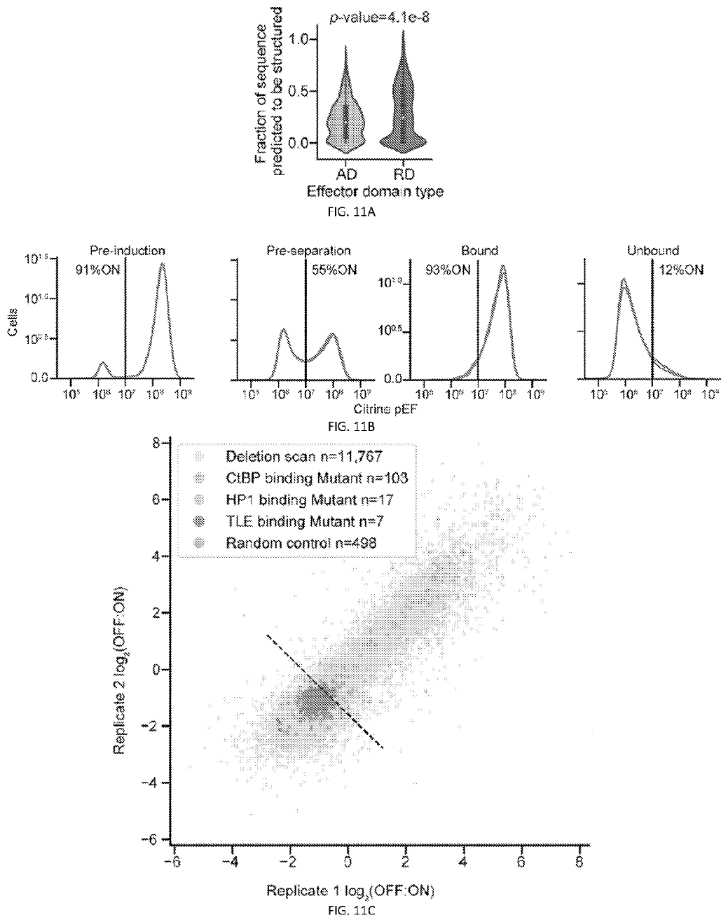

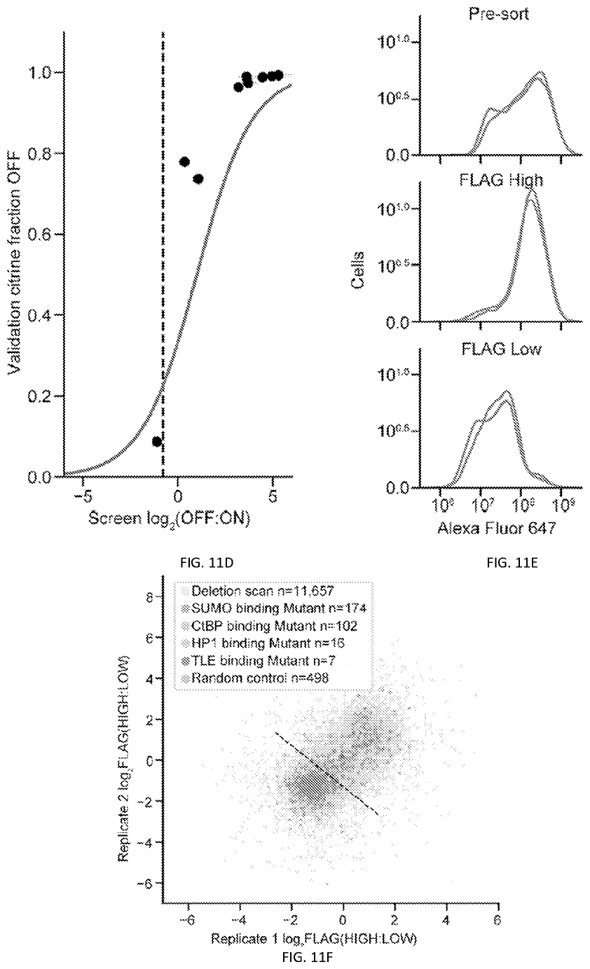

For all of the screens, domains with <20 counts in both conditions of a given replicate were filtered out of downstream analyses. Hit thresholds varied across screens, depending on coverage, separation purity, and bio-replicate reproducibility, and were set based on: 1) the scores of negative controls, and 2) the validation curves relating screen scores to fractions of cells with the reporter ON or OFF as measured by flow cytometry for individual points. These validation curves are plotted for each screen (FIGS. 1G and 1I for the CRTF tiling screens, FIGS. 7E-7F for the hit validations screens, and FIGS. 9E and 11D for the mutant screens). The threshold was chosen to be 1-3 standard deviations away from the mean of poorly expressed random controls, with the exact number of standard deviations chosen to maximize the number of true positives and minimize the number of false positives across the validations. Noisier screens, with lower reproducibility, had higher hit thresholds in order to avoid false positives. For the expression screens, well-expressed tiles were those with a log2(FLAGhigh:FLAGlow) 1 standard deviation above the median of the random controls. For the CRTF tiling repressor screens, hits were tiles with enrichment scores 3 standard deviations above the mean of the poorly expressed random controls. For the minCMV CRTF tiling, pEF bifunctional deletion scan, and minCMV bifunctional deletion scan screens, hits were proteins with enrichment scores 2 standard deviations above the mean of the poorly expressed random controls. For the validation and mutant screens, hits were proteins with enrichment scores 1 standard deviation above the mean of the poorly expressed random controls.

Annotation of domains from tiles Tiles must have been hits in both the CRTF tiling and validation screens in order to have been considered potential EDs. A domain started anywhere the previous tile was not a hit. If the previous tile was not a hit because it was not expressed, and if the antepenultimate (previous, previous) tile was a hit, then that tile was not considered the start, and instead it was recovered into the middle of the domain. A domain ended anywhere the next successive tile was not a hit. If the next tile was not a hit because it was not expressed, and the following tile was a hit, then the tile that was not expressed was not considered the end. Domains started at the first residue of the first tile and extended until the last residue of the last tile within the domain. Single tiles that were hits in both the CRTF tiling and validation screens were considered EDs. For example, AKAP8's single activation tile, had activity when recruited individually, and its corresponding tile in the Mutant AD screen contains deletions of unnecessary regions that maintained activation.

Individual recruitment assays and flow cytometry measurements Protein fragments were cloned as a fusion with rTetR upstream of a T2A-mCherry-BSD marker, using GoldenGate cloning in the backbone pJT126 (Addgene #161926). K562 citrine reporter cells were then transduced with each lentiviral vector and, 3 days later, selected with blasticidin (10 mg/mL) until >80% of the cells were mCherry positive (6-9 days). Cells were split into separate wells of a 24-well plate and either treated with doxycycline (Fisher Scientific) or left untreated. Time points were measured by flow cytometry analysis of >10,000 cells (Biorad ZE5, Everest version 2.3-3.0). Doxycycline was assumed to be degraded each day, so fresh doxycycline media was added each day of the timecourse.

Flow cytometry analysis Data were analyzed using Cytoflow (version 1.1, github.com/bpteague/cytoflow) and custom Python scripts. Events were gated for viability and mCherry as a delivery marker. To compute a fraction of ON cells during doxycycline treatment, a Gaussian model was fit to the untreated rTetR-only negative control cells which fits the OFF peak, and then set a threshold that was 2 standard deviations above the mean of the OFF peak in order to label cells that have activated as ON. The same was done for computing the fraction of OFF cells in repressor validations but a two component Gaussian was fit and a threshold that was 2 standard deviations below the mean of the ON peak was set. A logistic model, including a scale parameter, was fit to the validation and screen data using SciPy's curve fit function.

CRISPR HT-recruit to measure transcriptional effectors at endogenous genes HT-recruit screens were performed with dCas9 as the DBD and an sgRNA targeting either a lowly-expressed or highly-expressed endogenous surface marker (CD2 or CD43). First, the sgRNA was stably delivered to K562 cells by lentivirus and selected with puromycin for 3-4 days. The cells were confirmed to be >95% mCherry+ by flow cytometry (Accuri).

For the dCas9-CRTF screens, lentivirus for the library was generated using 16×15 cm dishes of HEK293T cells and then concentrated 4× using LentiX. Then 1.15×10⁸ K562-sgRNA cells per replicate were infected with 72 mL of the lentiviral library by spinfection for 2 hours, with two separate biological replicates of the infection, resulting in 18-23% BFP+ cells in unselected cells after 4 days. 2 days after infection, the cells were selected with 10 ug/mL blasticidin (InvivoGen). Cells were >95% BFP+ by the final timepoint. On day 11 post-infection, 5×10⁸ cells (>3,000×coverage) were taken for magnetic separation and measurement.

For dCas9 HT-recruit screens, cells were stained with antibodies against the target surface marker before magnetic separation. Cells were first washed with 1% BSA (Sigma) in 1×DPBS (Life Technologies) and spun down and supernatant was aspirated without disturbing the pellet. 5 mL of cells were then incubated on ice for 1 h with fluorophore conjugated primary antibody. The following primary antibodies were used: 100 ul of allophycocyanin (APC)-labeled anti-CD2 antibody (130-116-253, Miltenyi-Biotec) or 10 ul of APC-labeled anti-CD43 antibody (clone 4-29-5-10-21, eBioscience, Catalog #17-0438-42). Afterwards, cells were washed with 45 mL of 1% BSA/DPBS. They were then magnetically separated with Protein G Dynabeads as described for the rTetR screens.

Western blots Twenty million cells were pelleted and washed 1× with 5 mL of PBS. Pelleted cells were resuspended in 500 uL of ice cold lysis buffer (1×RIPA (EMD Millipore 20-188), 1% Triton X-100, 0.1% SDS, Roche complete protease inhibitor cocktail mini tablet) and were put on a rotator at 4 C for 30 minutes. Next, the lysates were sonicated with a COVARIS ultra-sonicator for 15 minutes (Peak power: 140-175, Duty factor: 10, Cycles/burst: 200). Lysates were spun down at 20,000 g for 5 minutes. Protein amounts were quantified using the Qubit protein broad range assay kit (Thermo Scientific, #A50668). 30 ug were denatured in 1×laemmli sample buffer (Bio-rad #1610747) +10% 2-mercaptoethanol for 10 minutes at 70 C and subsequently loaded onto a gel and transferred to a PVDF membrane. Membrane was first blocked with 7% nonfat dry milk (Bio-rad #1706404) for 1 hour at room temperature, then probed using FLAG M2 monoclonal antibody (1:1000, mouse, Sigma-Aldrich, F1804) and Histone 3 antibody (1:2000, rabbit, Abcam, AB1791) as primary antibodies overnight. Next, the membrane was washed with TBS-T 3×, 5 minutes each before being blotted again with goat anti-mouse IRDye 680 RD (1:20,000) and goat anti-rabbit IRDye 800CW (1:40,000, LICOR Biosciences, cat nos. 926-68070 and 926-32211, respectively) secondary antibodies for one hour at room temperature. Blots were imaged on a Licor Odyssey CLx imager. Band intensities were quantified using ImageJ's gel analysis routine.

Data analysis and statistics All statistical analyses and graphical displays were performed in Python58 (v. 3.8.5). Enrichment scores shown in all figures (aside from replicate plots) are the average across two separately transduced biological replicates. The p-values, statistical tests used, and n are indicated in the figure legends.

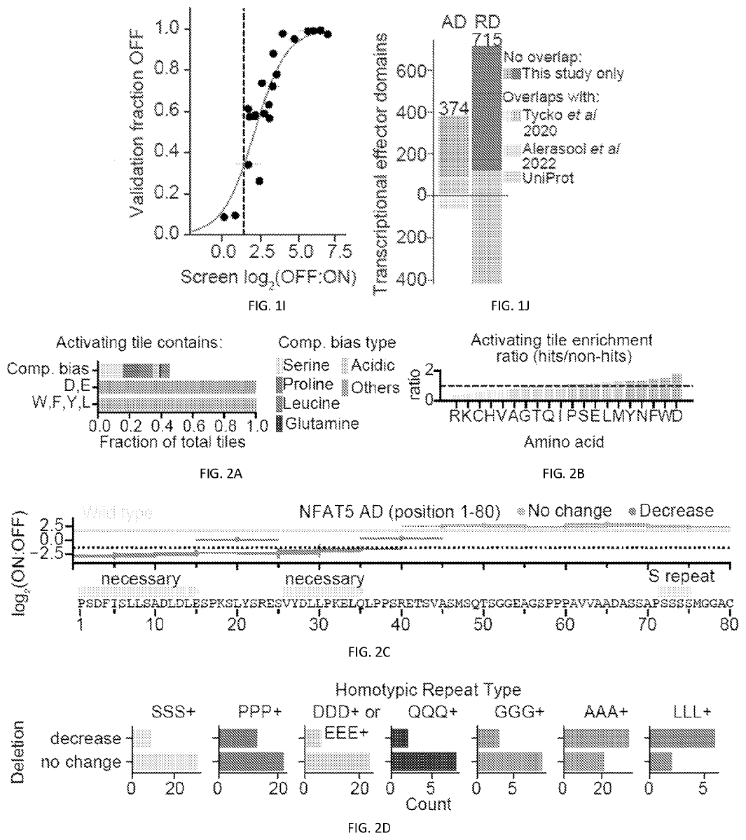

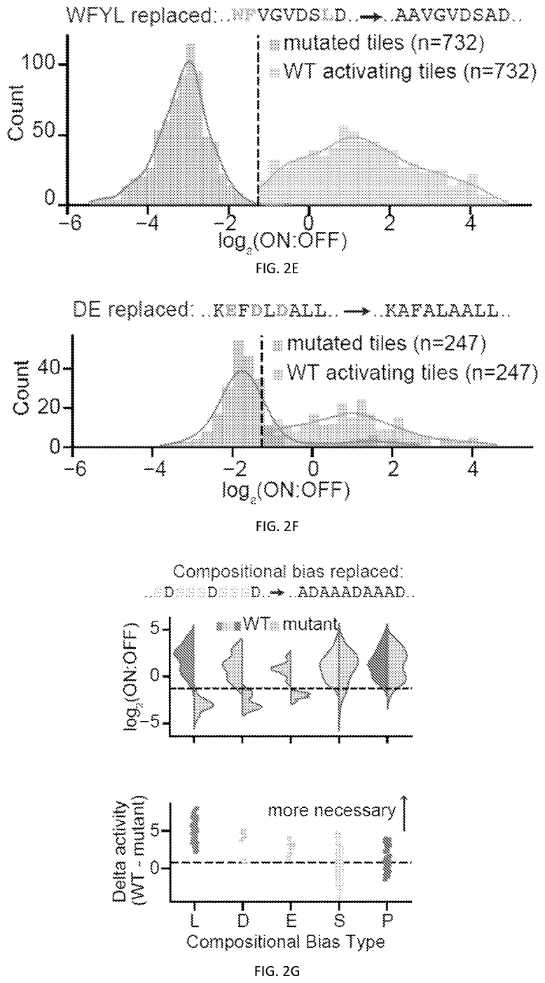

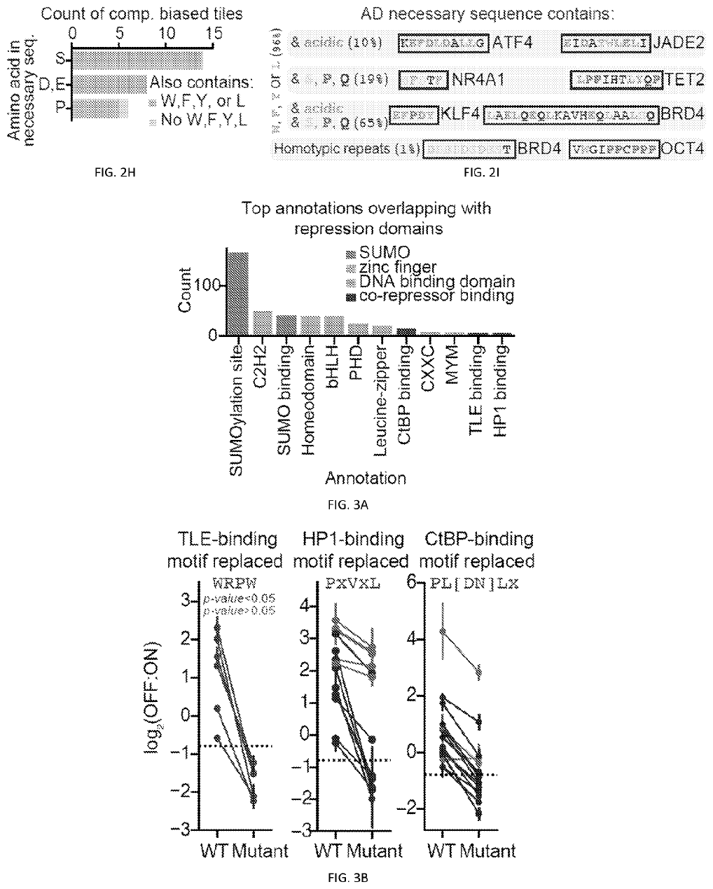

Protein sequence analysis Compositional bias was defined as an aa that appeared at least 12 times in 80 aa (e.g., 15% of the sequence). In FIG. 2B, for each aa, a ratio was computed by counting the abundance of each aa in the tile and normalizing by the length and total number of sequences. Randomly sampled 10,000 non-hit 80 aa sequences were similarly calculated and the enrichment ratio was calculated by dividing the hits by non-hits. For the few activation tiles that contained glycine-rich and glutamine-rich sequences, there were fewer than 5 mutants that expressed well as measured by FLAG and these were excluded from further statistical analyses.

Code availability The HT-recruit Analyze software for processing high-throughput recruitment assay and high-throughput protein expression assays are available on GitHub (github.com/bintulab/HT-recruit-Analyze).

### Example 1

### High-Throughput Mapping of Effector Domains (EDs)

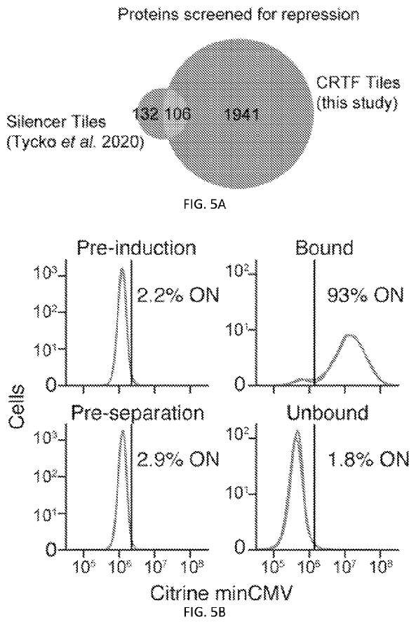

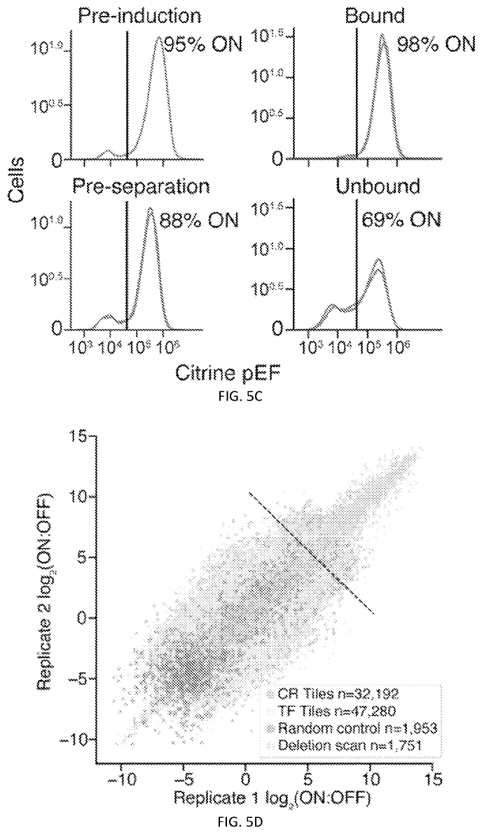

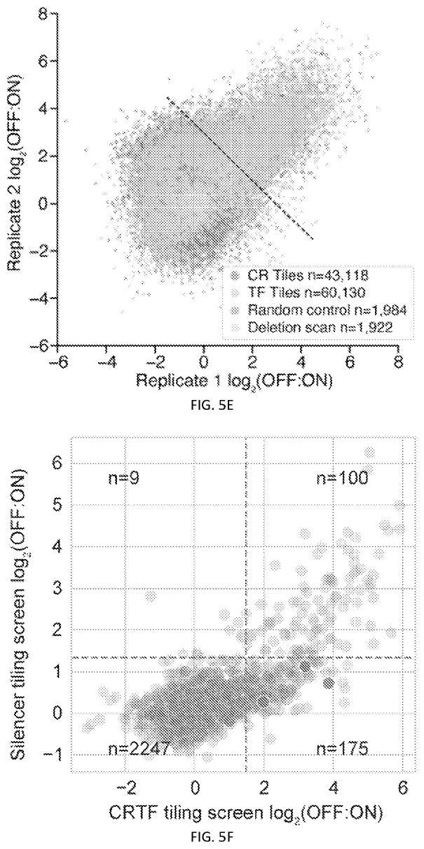

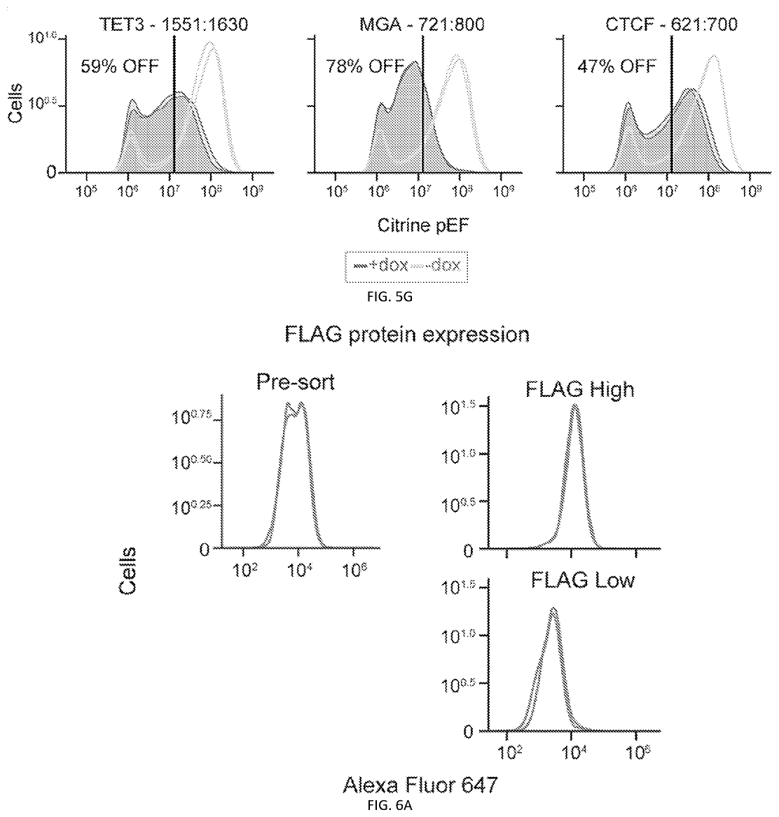

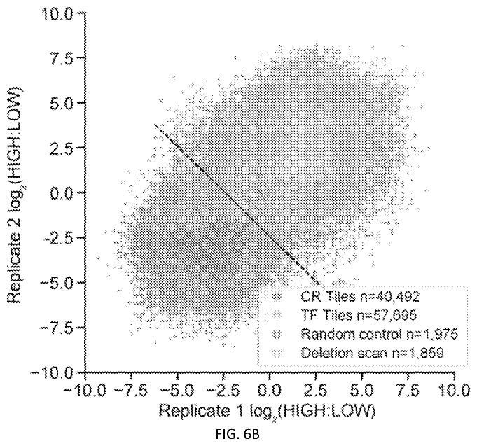

To map the human EDs at unprecedented scale and resolution, DNA sequences encoding 80 amino acid (aa) segments that tile across 1,292 human transcription factors (TFs) and 755 chromatin regulators (CRs) (hereafter CRTF tiling library) with a 10 aa step size between segments were synthesized (FIGS. 1A and 5A). This library, consisting of 128,565 sequences, was cloned into a lentiviral vector, where each protein tile was expressed as a fusion protein with rTetR (a doxycycline inducible DNA binding domain), and delivered as a pool at a low lentiviral infection rate, such that each cell contained a single rTetR-tile, to K562 cells containing a reporter with binding sites for rTetR. The reporter consisted of a synthetic surface marker that allows facile magnetic separation of cells for high-throughput measurements, and the fluorescent protein citrine for flow cytometry quantification during individual validations. The reporter gene was driven by either a minimally active minCMV promoter for identifying activators, or constitutively active pEF promoter for finding repressors. To simultaneously measure the effector function of these sequences, a recently developed high-throughput recruitment assay, HT-recruit, was used (See, Tycko, J. et al. Cell 183, 2020-2035.e16 (2020), incorporated herein by reference in its entirety). After treating the cells with doxycycline, which recruits each CRTF tiling library member to the reporter, the cells were magnetically separated into ON and OFF populations and the tiles were sequenced to identify sequences enriched in each cell population (FIGS. 5B-5C). Each screen was reproducible across two biological replicates (FIGS. 5D-5E). Thresholds for calling hits were based on the scores of random negative controls (FIGS. 5D-5E). 90% and 92% of the positive control domains for activation and repression, respectively, were hits above this threshold. Among the tiles shared with the previous screen, an additional subset of tiles that were only hits in this repression screen and whose activity validated in individual flow cytometry experiments were identified (FIGS. 5F-5G). Overall, these results demonstrated HT-recruit reliably identified EDs while using an order-of-magnitude larger library than the previous screen.

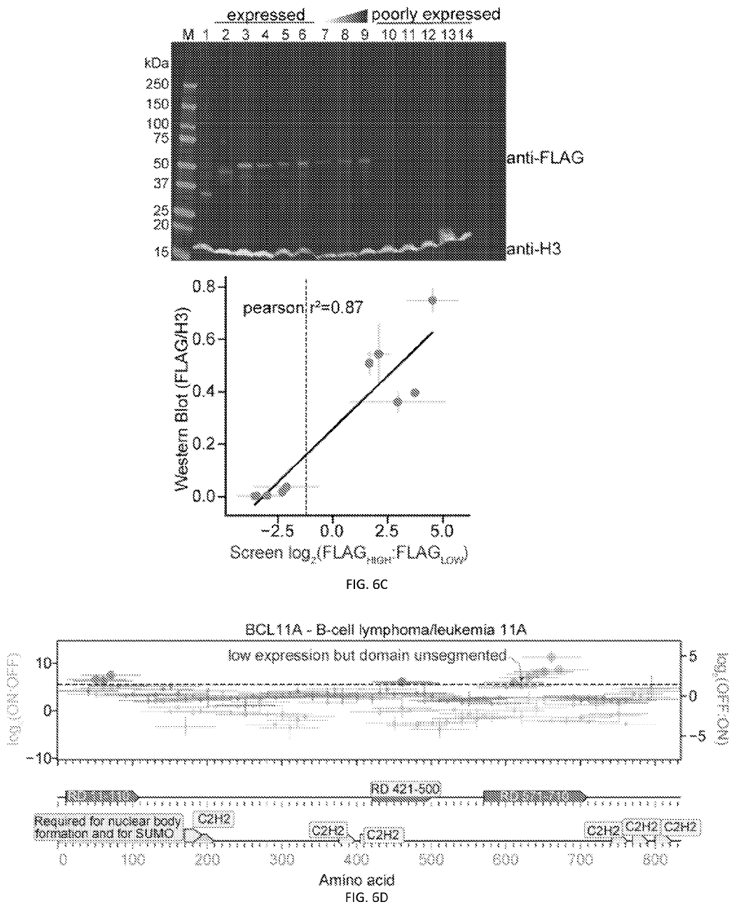

Measured transcriptional strength depends not only on the intrinsic potential of the sequence but also on the levels at which individual tiles are expressed. All library members contain a 3xFLAG tag, allowing measurement of each fusion protein's expression levels by staining with an anti-FLAG antibody, FACS sorting the cells into FLAG HIGH and LOW populations (FIG. 6A), and measuring the abundance of each member in the two populations by sequencing the domains (FIG. 6B). These FLAG scores from the high-throughput measurements can identify proteins that are not expressed, as determined from individual validations using Western blotting (FIG. 6C), and were used when annotating EDs, allowing filtering out of false negative library members that have lower activation or repression scores due to low expression (FIG. 6D).

To further confirm all the hits and help remove false positives, a smaller library containing only the activating and repressive hit tiles was screened (hereafter validation screen). Because of their small size, these screens had better separation purity (FIGS. 7A-7B) and could be screened at 10-fold higher coverage, which resulted in higher reproducibility than the original, larger screens (FIGS. 7C-7D), and even better correlation between screen scores and individual validations (FIGS. 7E-7F). About 80% of the hits were confirmed as hits in these validation screens (FIGS. 7C-7D). These confirmed sequences were those considered in subsequent analyses.

Using these filtered tiling data, EDs from contiguous hit tiles were annotated (FIGS. 1B, SEQ ID NOs: 31, 36, 111, 113, 153, 158, 165, 182, 184, 189, 224, 291, 311, 313, 352, 362, 367, 369, 375, 381, 407, 410, 415, 426, 430, 436, 472, 476, 478, 480, 483, 487-489, 494, 496, 498, 509, 512-517, 524, 526, 527, 530, 532, 533, 537, 541, 542, 545-547, 549, 552, 554, 557, 560-562, 565-568, 570-576, 578, 579, 580, 581, 582, 585, 587, 589, 590, 592, 595-598, 601, 603, 605, 607, 613, 617, 620, 622-624, 626, 627, 629, 630, 634-636, 639, 643, 646, 648, 651, 654, 658, 659, 662, 664, 666, 673, 675, 677, 678, 681, 684, 685, 686, 687, 689, 695, 696, 697, 699, 704, 705, 707-711, 713, 715, 716, 721, 723-725, 728, 729, 731-733, 735, 744, 746, 747, 753, 755, 760, 761, 764, 766-769, 773, 775-984, 1036, 1054, 1055, 1069, 1120, 1144, 1182, 1183, 1200, 1208, 1314, 1318, 1366, 1402, 1417, 1442, 1516, 1518, 1543, 1598, 1627, 1655, 1665, 1667, 1670, 1706, 1710, 1711, 1735, 1738, 1742, 1747, 1748, 1752, 1756, 1763, 1777, 1783, 1786, 1789, 1793, 1794, 1808, 1811, 1822, 1831, 1838, 1839, 1854, 1859, 1862, 1865, 1866, 1869, 1870, 1872, 1875, 1883, 1889, 1891, 1893, 1901, 1902, 1905, 1907, 1910, 1912, 1913, 1914, 1915, 1916, 1922, 1923, 1927, 1930, 1934, 1940, 1944, 1946, 1948, 1951, 1952, 1956, 1957, 1968, 1969, 1972, 1987, 1992, 1994, 1996, 2004, 2007, 2010, 2017, 2022, 2029, 2033, 2041, 2042, 2043, 2048, 2050, 2051, 2053, 2057, 2064, 2095, 2107, 2112, 2119, 2123, 2128, 2131, 2139, 2150, 2157, 2160, 2163, 2176, 2182, 2188, 2190, 2192, 2193, 2194, 2205, 2206, 2207, 2208, 2211, 2212, 2213, 2216, 2218, 2221, 2224, 2227, 2231, 2232, 2239, 2245, 2246, 2254, 2262, 2263, 2265, 2271, 2274, 2275, 2277, 2278, 2282, 2283, 2288, 2292, 2295, 2296, 2298, 2302, 2312, 2313, 2316, 2320, 2321, 2323, 2324, 2325, 2334, 2338, 2341, 2348, 2361, 2364, 2365, and 2370-6094), resulting in accurately identifying EDs previously annotated in UniProt, for example MYB's EDs (FIG. 1B). Some of the strongest EDs come from gene families with some family members already annotated as activators (e.g., ATF and NCOA) and repressors (e.g., KLF and ZNF), increasing confidence in the screen (FIGS. 1C and 1D). TFs from certain gene families (e.g., KLF and KMT) contain both strong activation domains (ADs) and repression domains (RDs), which highlights the results can identify bifunctional transcriptional regulators. In total, 12% of the proteins screened were bifunctional and 77% of proteins had at least one ED.

In addition, this method facilitated discovery of previously unannotated EDs (FIG. 1E). For example, a new AD and four new RDs were found within the DNA demethylating protein, TET2. Tens of these new EDs were validated by individually cloning them, creating stable cell lines, and measuring their effect using flow cytometry after dox-induced recruitment (FIGS. IF and 1H). In these experiments, fluorescence distributions are often not unimodal, most likely due to stochastic gene expression: bursting in the case of activation and stochastic silencing in the case of repression. These results were used to validate screen thresholds: all tiles above the thresholds had activity and no tiles below did (FIGS. 1G and 1I).

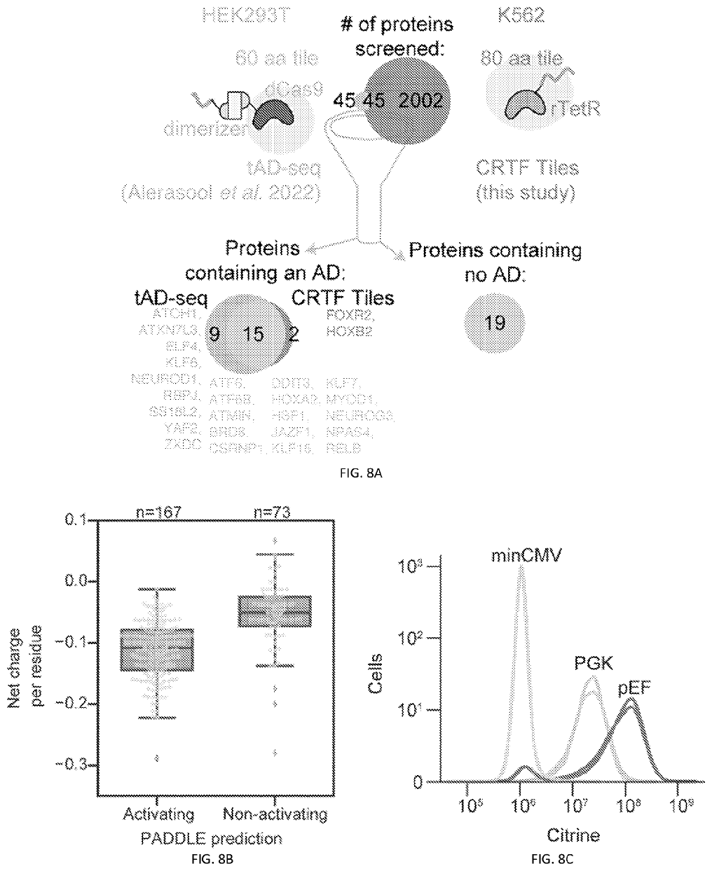

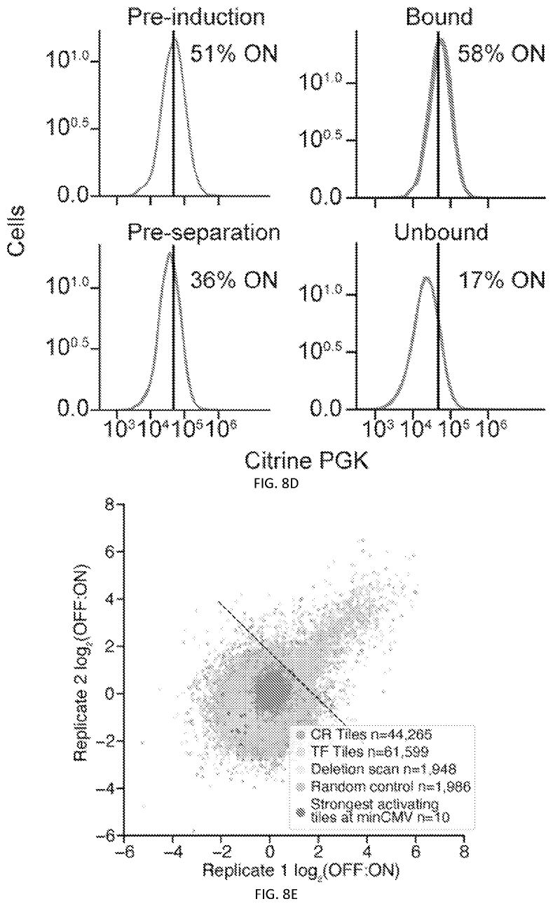

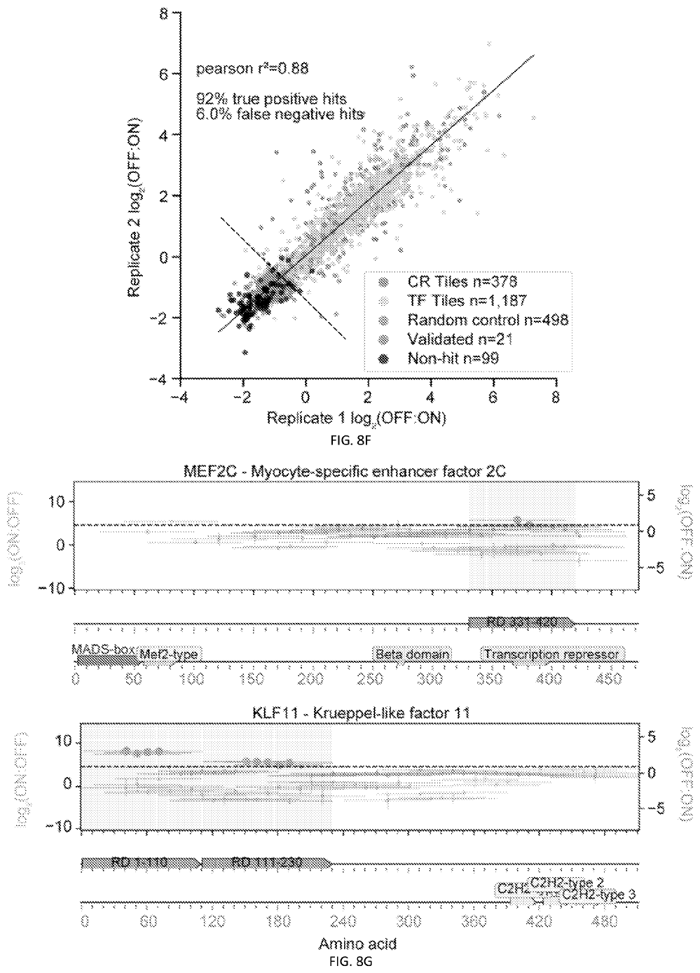

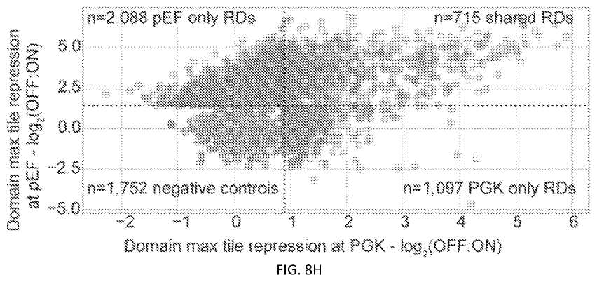

Forty-five of the proteins tiled here were recently screened for activation in HEK293T cells, but tiled with smaller fragments. The two studies showed good agreement: 19 proteins did not activate in both screens, and 15 proteins did (FIG. 8A). The proteins that only activated in one of the studies could represent activators that are unique to the specific context (cell type for example) but could also reflect the difference in length. For example, KLF6 tiles that only activated with smaller fragments overlapped a RD in the measurements with longer tiles. While longer tiles can possibly capture large ADs, shorter peptides are more likely to find small ADs that are near RDs.

Prior screens in yeast have led to the development of a machine learning model (PADDLE12) capable of predicting activation levels from sequence alone with an area under the precision-recall curve of 81%. If the sequence properties that drive activation in humans are like those in yeast, PADDLE would be expected to predict human ADs with similar accuracy. While PADDLE was able to predict 70% of the ADs, the domains that PADDLE predicted to be activating were more negatively charged than the ADs it missed (FIG. 8B), suggesting that in human cells there are additional non-acidic activator classes compared to yeast.

Because there are no other comprehensive studies in human cells or predictive models with which the RDs can be compared, the repressive measurements were repeated with the entire CRTF library at a second promoter: PGK. While this promoter is weaker (FIG. 8C), silent and active cells were able to be magnetically separated (FIG. 8D) and good reproducibility was observed (FIG. 8E). Ninety-two percent of the hit tiles that showed up in the pEF and PGK screens also showed up as hits in the pEF validation screen (FIG. 8F), suggesting higher confidence results when both screens were combined. Taking the maximum tile's enrichment scores within each RD revealed 715 RDs were shared across both screens (FIGS. 8G-8H). Together, these results suggested that at the 80 aa scale there are more sequences across the CRs and TFs that can work as repressors versus activators. In total, 291/374 ADs and 592/715 RDs are new compared to previous annotations (FIG. 1J).

### Example 2

### Activation Domain (AD) Characterization

The large set of new ADs provides a great opportunity to systematically quantify the prevalence of sequence properties e.g., abundance of particular amino acids such as acidic, glutamine-rich, and proline-rich sequences, homotypic repeats, and enrichment of particular hydrophobic residues—aromatics (W, F, Y) and leucines (L). Forty-five percent of activating tiles contained a compositional bias (FIG. 2A), where serine and proline are the most abundant. Consistent with these observations, when the aa frequencies in the AD sequences were further normalized by the non-hit sequences, there was an enrichment in certain hydrophobic, acidic, serine, and proline residues (FIG. 2B).

Despite being well-documented, very few Q-rich ADs were identified (FIG. 2A, n=10). Annotated Q-rich ADs are longer than 80 aa, thus the tiling approach might have missed them. Alternatively, Q-rich ADs could be relatively weak, and utilize other TFs to activate. Recruitment of SP1's two annotated Q-rich ADs25 (longer than 80 aa) did not activate minCMV (FIG. 9A). However, including a short, acidic AD upstream of the Q-rich domains was sufficient for SP1's “tAD A” to activate (FIG. 9A). This result supp.orts the previous observations that acidic and Q-rich domains work synergistically in human cells.

To determine which amino acids facilitated activation, a deletion scanning approach was used: the activity of mutant ADs containing consecutive small deletions was measured (FIG. 9B, top). Although most (61%) deletions do not affect activation, at least one deletion was found that was well-expressed and could abolish activator function in most of the pilot ADs (20/24 with activity at minCMV). To confirm whether this approach could resolve residues facilitating activity, the deletion scan data from P53 was compared to UniProt and residues 20-22 (DLW) found within one region and residue W52 found within another facilitated activity, corresponding to UniProt-annotated TAD I and TAD II (FIG. 9B, top). Furthermore, individual validations of deletions including these residues confirmed complete loss of activity (FIG. 9B, bottom).

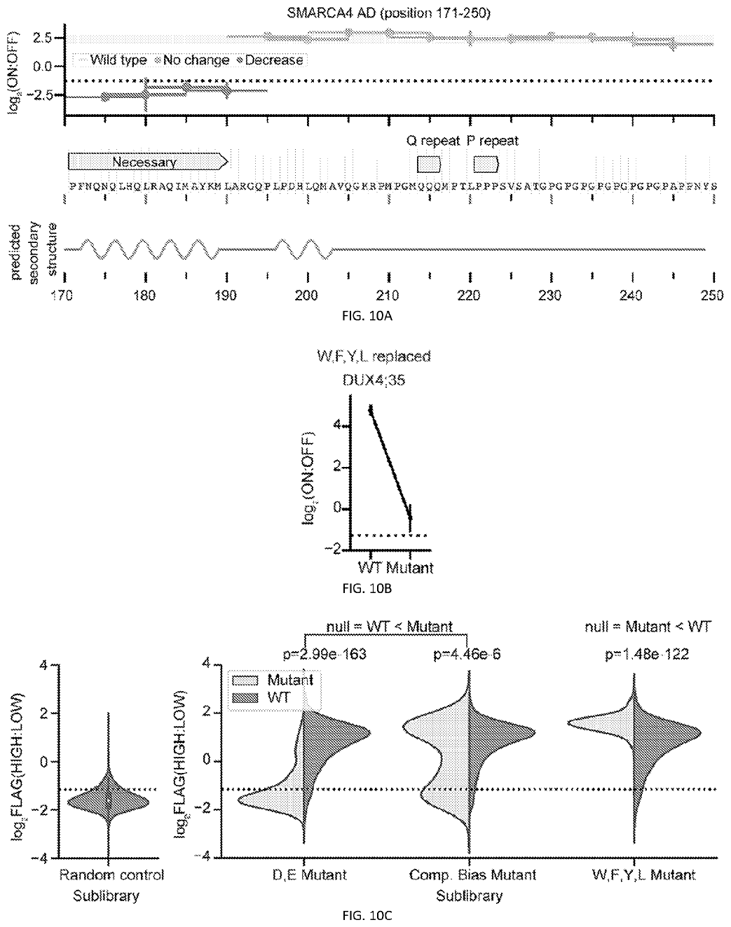

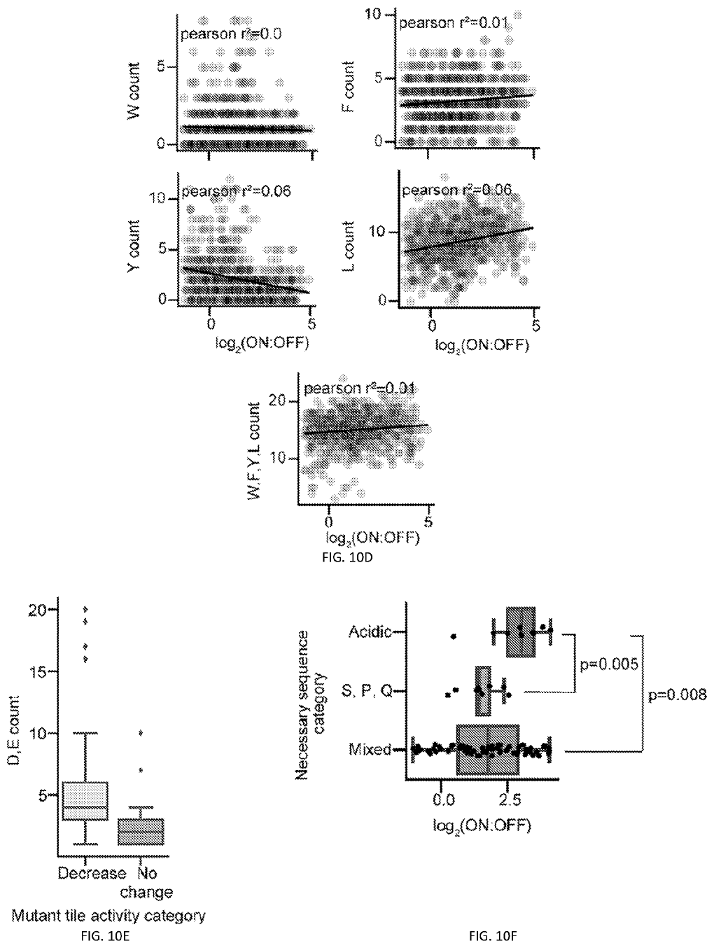

Confident in the deletion scan approach, a second library of 10 aa deletions across the maximum activating tile from each AD was designed, resulting in 304 total deletion scans. Activation was measured using the minCMV reporter and HT-recruit workflow described in FIG. 1A (FIGS. 9C-9E) and mutants that were poorly expressed were filtered out based on FLAG-staining (FIGS. 9F-9G). Across each of these expression-filtered deletion scans, deletions were classified according to their effect on activation (FIG. 2C). Using these data, it can be determined which compositionally biased residues are important for function and which are not: for example, while NFAT5's AD has a patch of 4 serines near the C-terminus, deleting those residues had no effect on activation (FIGS. 2C and 10A). Applying this analysis to all ADs containing a homotypic repeat, serine, proline, acidic, glutamine, and glycine homotypic repeats were more often found in deletions that had no effect on activation than in deletions that decreased activation (FIG. 2D). Therefore, homotypic repeats of these amino acids are generally not necessary for activation.

The deletion scans also identified the sequence for activation of each tile: sequences that, once removed, completely abolished activation (FIG. 2C). At least one sequence (median length=10 aa) was able to annotated in the majority (69%) of the screened ADs, and most (61%) ADs had multiple sequences (FIG. 2C, see, for example SEQ ID NOs: 17424-17841). Nearly every sequence (96%) contained a W, F, Y or L.

To validate this enrichment of specific hydrophobic residues, mutant libraries were rationally designed where every aa of a particular type within the sequence was systematically replaced with alanines (See, for example, SEQ ID NOs: 13274-17423). Replacement of all W, F, Y or Ls with alanine (range: 3-24 aa replaced/80 aa tile, median=10 aa) in all the activating tiles resulted in a total loss of activation (FIG. 2E). The one exception that remained active was within DUX4, and the mutation did make it weaker (FIG. 10B). This systematic loss of activation was not due to a decrease in protein expression, as measured by FLAG staining (FIG. 10C). There is no correlation between the overall count of these residues within tiles and a tile's activation strength (FIG. 10D), likely suggesting these residues mediate interactions for activity, and the placement of these residues is more important than the overall count. This means ADs from 258 different proteins utilize at least some aromatic or leucine residues to activate.

All acidic residues were replaced with alanine in all activating tiles. Surprisingly, more than half of the acidic mutants had reduced expression (FIG. 10C). These results suggested that the acidic residues increased protein levels, at least in the context of ADs. Of the remaining 247 well-expressed activating tile mutants, most mutants lost the ability to activate (FIG. 2F, n=196). The mutants with no change in activity had significantly fewer acidic residues than the tiles whose mutants had a decreasing effect (FIG. 10E), supporting the idea that acidic ADs are not the only class of human ADs.

Intrigued by what other compositional biases could be functional in human ADs, other frequently-appearing residues were replaced with alanine. Consistent with the results above, all tiles with leucine and acidic compositional biases lost activity once mutated (FIG. 2G). Removal of serine and proline compositional biases had more mild effects: most mutants still had activity (FIG. 2G, top), even though the strength of activation decreased for a subset of them (FIG. 2G, bottom).

Wanting to follow up more on the compositionally biased tiles that decreased activity upon compositional bias removal (FIG. 2G), the set of sequences (as determined from the deletion scans) from the compositionally biased activating tiles that lost activity upon bias removal were analyzed (FIG. 2G, bottom). For each bias type, most sequences also contain a W, F, Y, or L (FIG. 2H), suggesting their placement next to hydrophobic residues is important for their function.

In summary, sequences that facilitated activation consisted of certain hydrophobic residues (W, F, Y, and/or L) that are interspersed with either acidic, proline, serine, and/or glutamine residues (FIGS. 2I and 10F). Although prior work has shown that homopolymer stretches of glutamine and proline are sufficient to activate a weak synthetic reporter, it was found that the majority of glutamine and proline repeats within ADs of the human CRs and TFs are not part of the sequence for activation.

### Example 3

### Repression Domain Characterization

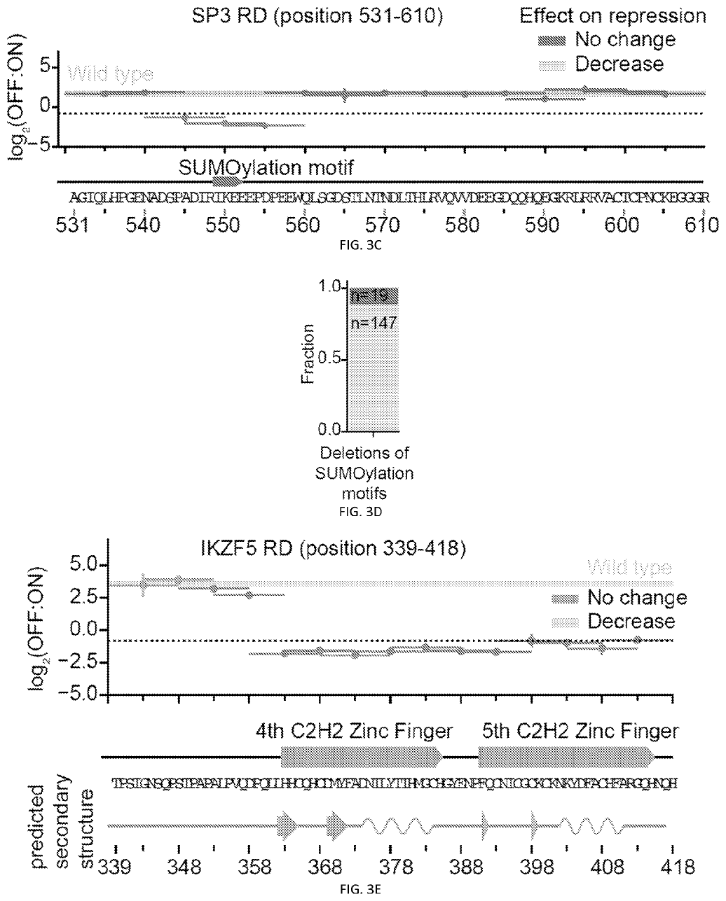

Repressing tile sequences have significantly more predicted secondary structure than activating tile sequences (FIG. 11A). Instead of looking at RD sequence compositions, RDs were first classified by their potential mechanism. The ELM database was used to search for co-repressor interaction motifs, and UniProt to search for domain annotations. Seventy-two percent of the RDs overlapped diverse annotations, such as sites for SUMOylation, zinc fingers, SUMO-interacting motifs, co-repressor binding motifs, DNA binding domains (including Homeodomains, consistent with previous results), and dimerization domains (FIG. 3A). To address whether these annotations facilitate repression, mutant libraries that replaced sections of 1,313 repressing tiles were rationally designed and this RD mutant library was screened using the pEF reporter and workflow described FIG. 1A (FIGS. 11B-11D). Additionally, protein expression was monitored (FIGS. 11E-11F) and mutants that had low FLAG enrichment scores were filtered out.

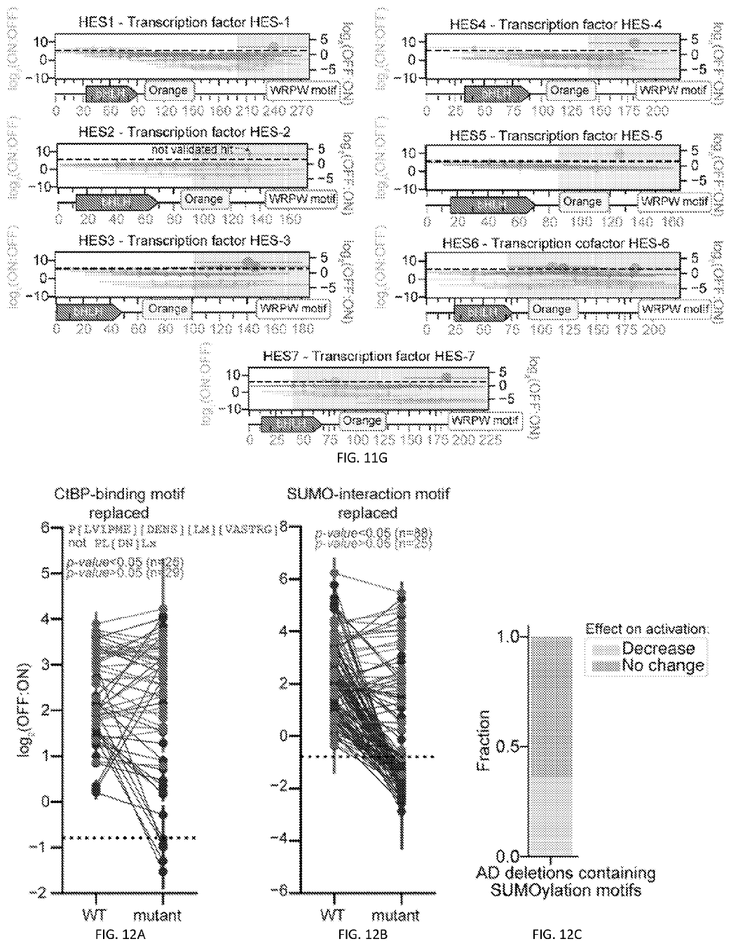

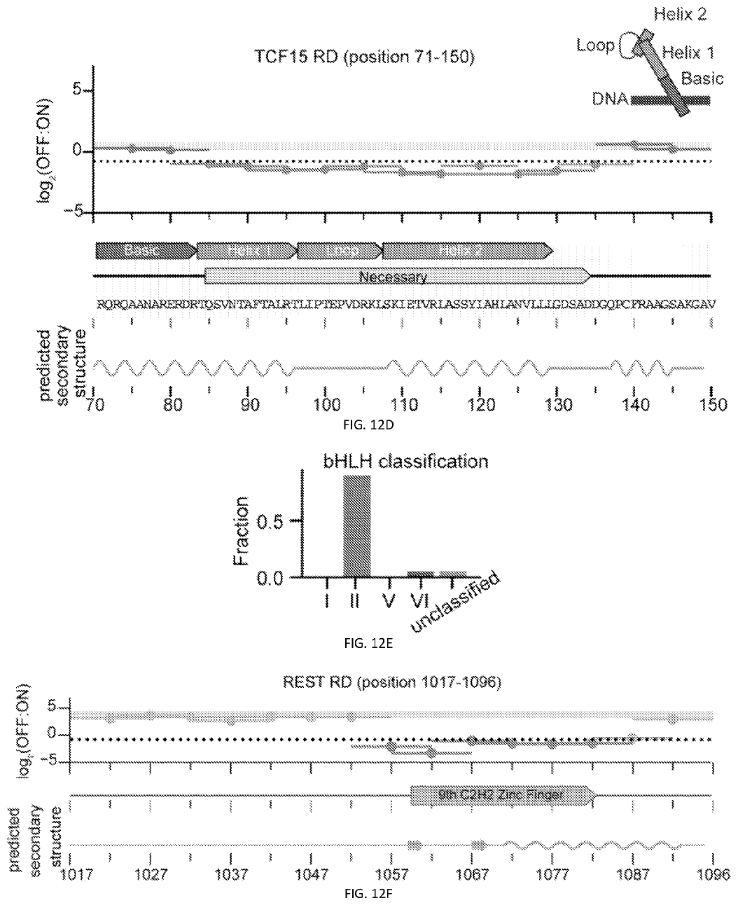

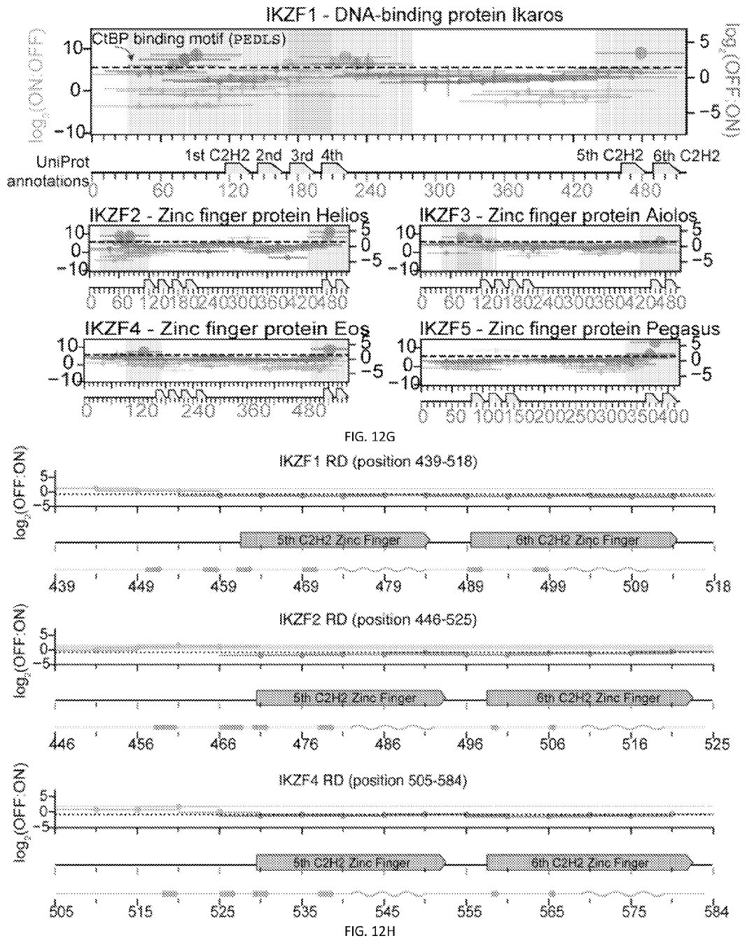

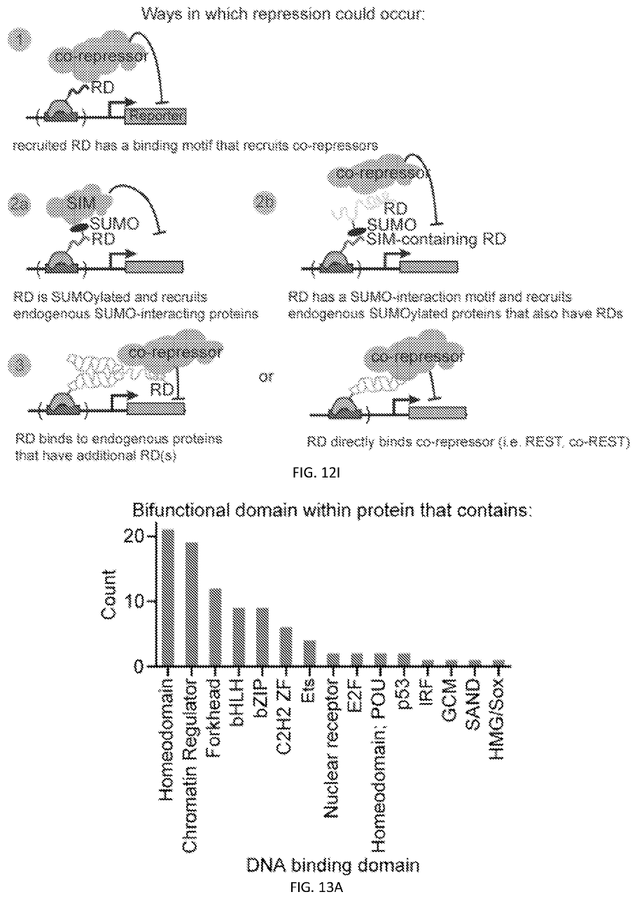

Co-repressor interaction motifs were systematically replaced with alanine to test their contribution to activity (FIG. 3B). The TLE-binding motif, WRPW (SEQ ID NO: 28212), appears exclusively in the C-terminal RDs of the HES family and all tiles containing this motif were repressive (FIG. 11G). All tested TLE-binding motifs facilitated repression (FIG. 3B, left). The HP1-binding motif, PxVxL, facilitated or contributed to repression in many of the tiles containing it (8/13 tiles with decreasing effects FIG. 3B, middle). A more refined CtBP motif explained most tiles that lost activity upon mutation (14/17 tiles FIGS. 3B, right, and 12A). Altogether, 78% of the 36 repressing tiles with a co-repressor binding motif (TLE, HP1, or CtBP) decreased in repression strength when the motif was mutated, and 78% of 113 SUMO interaction motif-(SIM, binding site to SUMOylated proteins) containing repressing tiles were similarly sensitive to mutation (FIG. 12B).

Many RDs contained a SUMOylation site (site for covalent conjugation of a SUMO domain) (FIG. 3A). The ELM database classifies SUMOylation sites with the search pattern φKxE. Because this motif is short and flexible, some non-hit sequences (12.3%) also contain SUMOylation motifs. To investigate whether SUMOylation sites within non-hit sequences are functional, the AD deletion scan data was used. Deleting a SUMOylation motif within ADs rarely decreased activation (FIG. 12C). The same deletion scanning approach was used to query if these motifs are functional in RDs (Supplementary Table 5, FIG. 3C). For example, residue K550 in the SP3 protein is a SUMOylation site and has been shown before to be important for repression; indeed this site was also found to overlap with the region for repression (FIG. 3C). In a similar manner, SUMOylation motifs were found to be important for the repression of at least 147 out of the 166 RDs where they are found (FIG. 3D). This result is concordant with a previous finding that a short 10 aa tile from the TF MGA, which contains this SUMOylation motif, IKEE (SEQ ID NO: 28213), is itself sufficient to be a repressor. SUMOylation of FOXP1 (which also shows up as a region in the results herein), has been shown to promote repression via CtBP recruitment. SUMOylation motif-containing TFs are enriched for binding co-repressor KMT2D, as reported in a bioID interaction resource (p-value=0.028, one-sided proportions z-test, compared to TFs with no EDs). A previously undescribed RD was also identified in KMT2D containing a SIM, suggesting SUMOylation for these TFs drives repression via SIM-containing co-repressor recruitment.

The deletion scan data was used to gain better resolution of the region within RDs overlapping dimerization domains, such as basic helix-loop-helix domains (bHLHs). Within bHLHs, the basic region binds DNA, and mutations in the HLH region are known to impact dimerization. Deletion scans across tiles that overlap HLH domains reveal part of helix 1, the loop, and helix 2 facilitate repression (FIG. 12D). HLHs lacking a basic region have previously been shown to negatively regulate transcription by forming complexes with other bHLHs and inhibiting their binding. Alternatively, as shown herein, bHLHs containing basic regions can negatively regulate transcription when recruited at a promoter, likely by forming functional dimer complexes with another bHLH from a TF that contains RDs elsewhere in the protein. The majority of RDs that overlap bHLHs belong to Class II tissue specific bHLH TFs (FIG. 12E) that can either activate or repress depending on the context. Indeed, bHLH TFs can act as activators in other contexts: for example, NEUROG3, a Type II bHLH TF, acts as an activator when recruited full length to the minCMV promoter and an activator tile was found that partially overlaps the bHLH RD. This context specificity to activation and repression of bHLH TFs might be expected given they can dimerize with different activating or repressing bHLH TFs.

Many RDs overlap annotated zinc fingers (ZFs, n=124), and some specifically overlap C2H2 ZFs (n=50, compared to only 3 ADs that overlap C2H2 ZFs p-value=5.9e-24, one-sided proportions z-test) (FIG. 3A). REST's 9th C2H2 ZF is repressive and directly recruits the co-repressor coREST. In agreement with these reports, the deletions in this RD of REST revealed the 9th ZF facilitates repression (FIG. 12F).

In addition to binding DNA and directly binding co-repressors, ZFs dimerize with other ZFs. ZFs could cause repression by binding to other ZF domains within endogenous repressive proteins, such as with the IKZF family where the N-terminus of some members, such as IKZF1, directly recruits CtBP, while the C-terminal zinc fingers bind other IKZF family members. Indeed, the N-terminal repressive domains in IKZF1 were recovered, and the associated sequence contained a CtBP binding motif (FIG. 12G). In addition, all IKZF family members showed C-terminal RDs that overlap the last two ZFs (FIG. 12G). These two ZFs both facilitated repression in IKZF5 (FIG. 3E) and in all tested family members (FIG. 12H), and therefore likely dimerize with the IKZFs that recruit CtBP. While in general ZFs are well-known DNA binding domains, the data show herein expands the list of ZF sequences that are likely protein binding domains to other repressive TFs.

In summary, RDs can be categorized in the following way: (1) domains that contain short, linear motifs that directly recruit co-repressors, (2) domains that contain SUMO interaction motifs or can be SUMOylated, or (3) structured binding domains that likely recruit co-repressors or other repressive TFs (FIGS. 3F and 12I).

### Example 4

### Bifunctional Activating and Repressing Domains

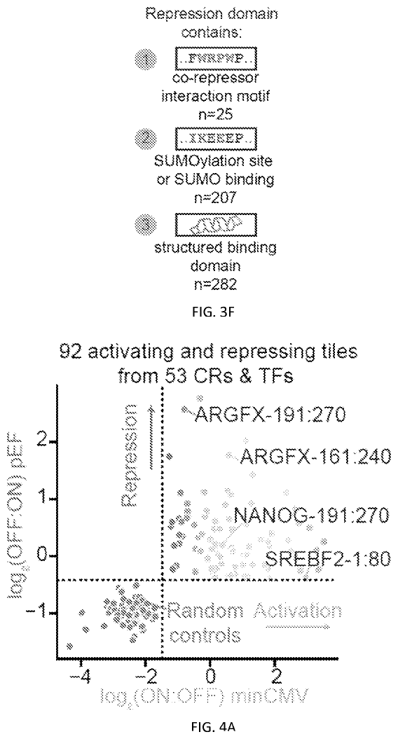

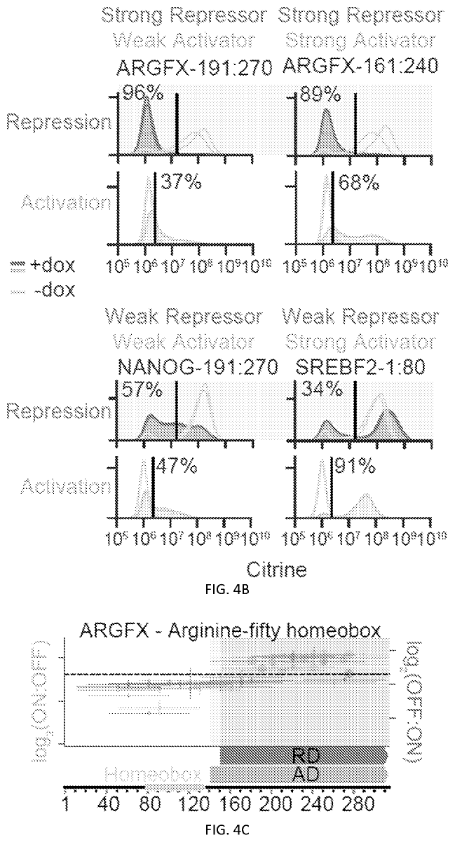

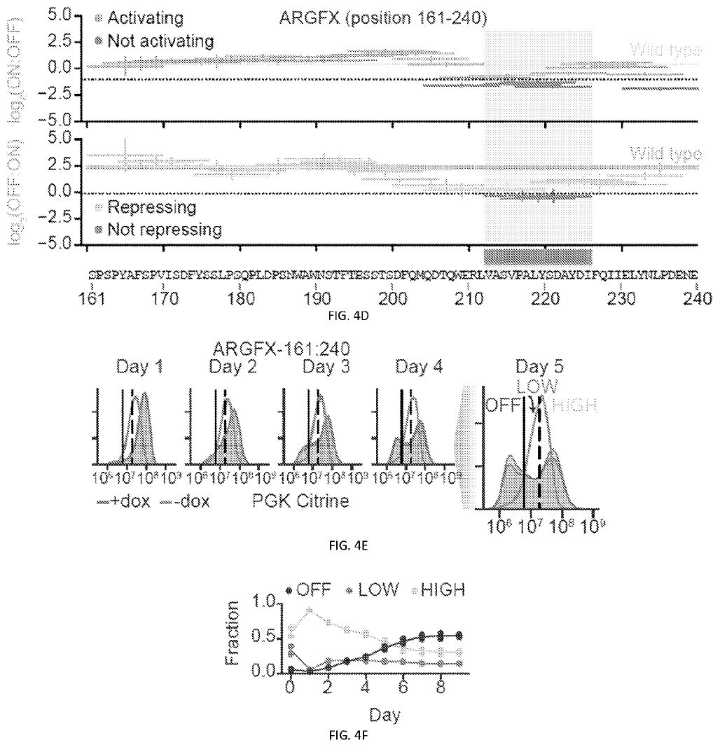

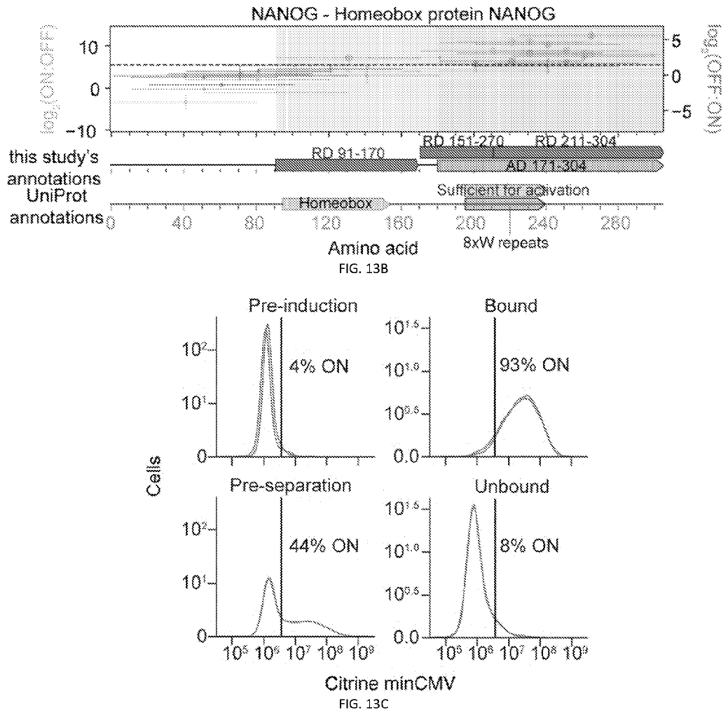

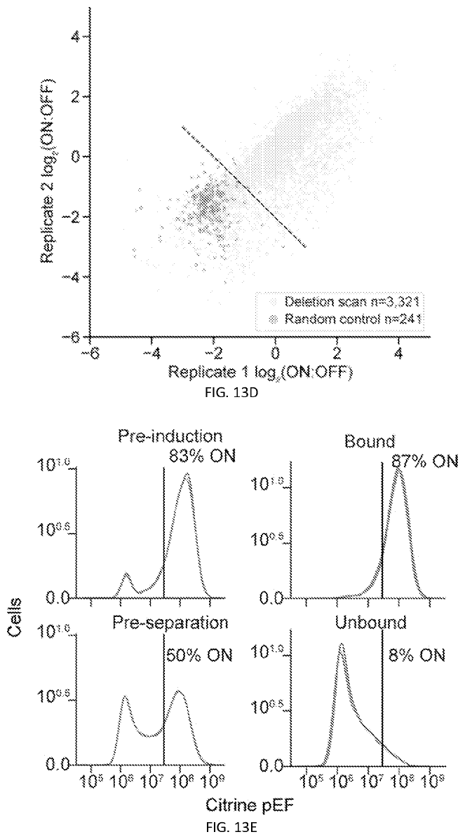

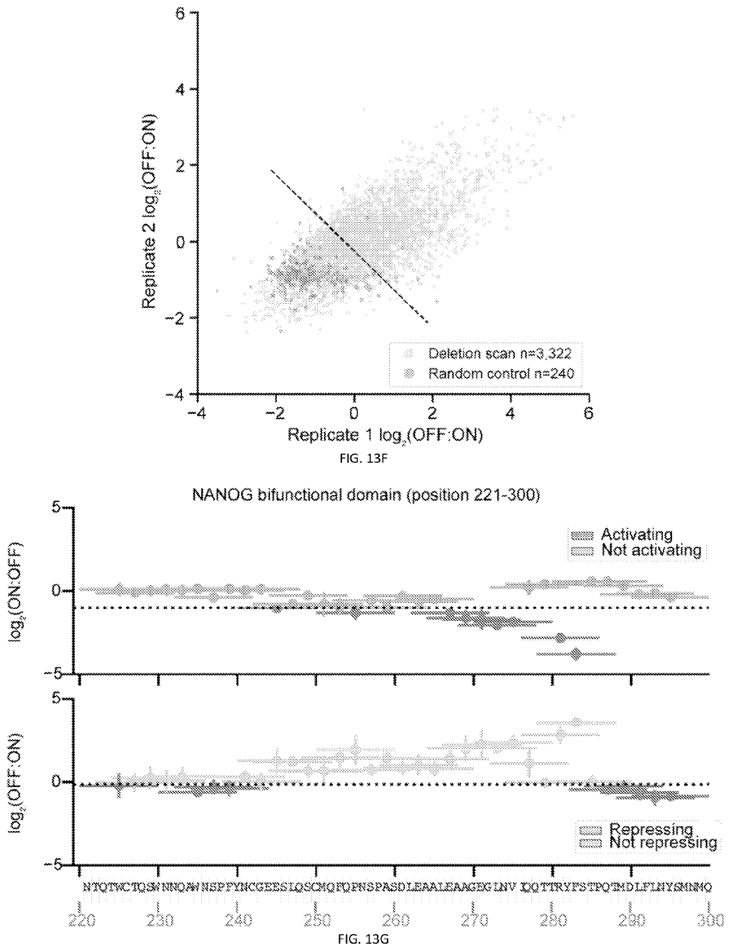

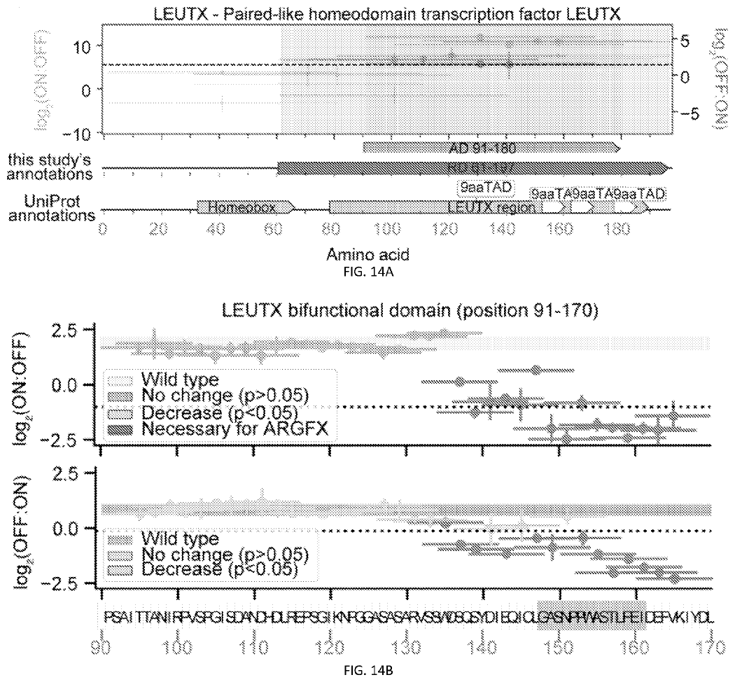

Transcriptional proteins are categorized as activating, repressing, or bifunctional, where 115 proteins have previously been found to activate some promoters but repress others. Here, 248 proteins are classified as bifunctional, CRs & TFs that have both an AD and RD (such as in FIG. 1B, SEQ ID NOs: 38, 40, 42, 55, 56, 57, 70, 75, 104, 105, 106, 109, 127, 129, 133, 134, 141, 142, 144, 145, 166, 167, 168, 180, 217, 227, 234, 235, 237, 238, 239, 240, 241, 250, 269, 271, 272, 273, 280, 281, 282, 283, 289, 299, 302, 303, 322, 323, 324, 325, 326, 327, 342, 343, 371, 377, 378, 400, 401, 403, 405, 411, 423, 431, 441, 453, 457, 475, 477, 483, 485, 496, 498, 528, 541, 562, 589, 610, 638, 646, 678, 694, 698, 704, 706, 711, 716, 738, 756, 757, 764, and 766). While most of these proteins contain both ADs and RDs at independent locations, a surprising fraction (92/248) possess single domains apparently capable of both activation and repression (FIGS. 4A-4C) with many found within homeodomain TFs (FIG. 13A).

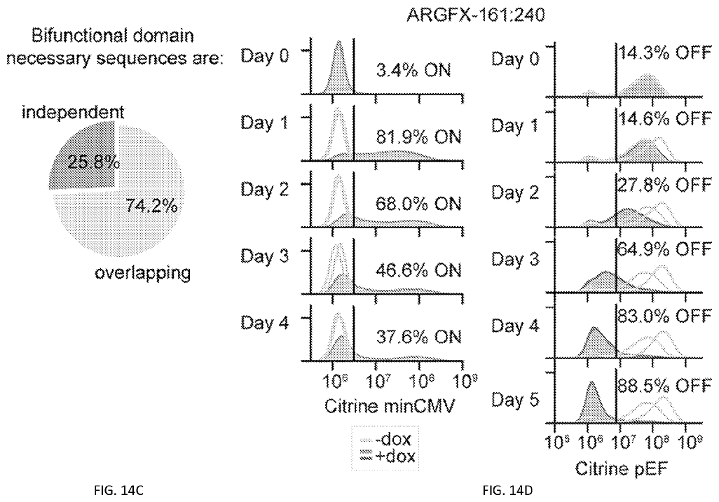

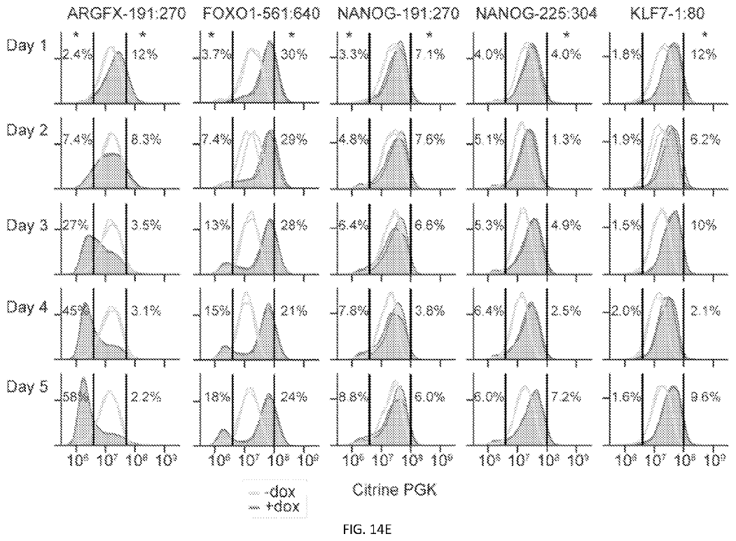

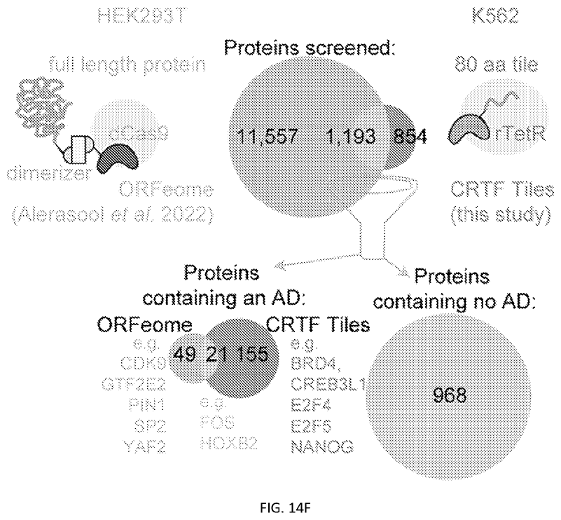

To further investigate their behavior, candidate bifunctional domains were individually recruited and doxycycline-dependent minCMV activation and pEF repression were quantified (FIG. 4B). These validation measurements recapitulated initial screen observations, highlighting some domains with similar strengths of both repression and activation (e.g., ARGFX-161:240 and NANOG-191:270), and others with preferential activities (e.g., ARGFX-191:270, SREBF2-1:80; FIGS. 4B and 13B). Entire bifunctional domains could drive activation or repression, or specific regions within domains could mediate distinct activities. Systematic deletions of 10 aa segments within bifunctional domains further refined the regions responsible for each activity (SEQ ID NOs: 25652-28198, FIGS. 13C-13F). While some bifunctional domains (23/92) possess independent activating and repressing regions (e.g., NANOG; FIG. 13G), others have fragments as small as 14 aa that can mediate both strong activation and repression (69/92 domains, e.g., ARGFX and the structurally related LEUTX) (FIGS. 4D, 14A-14C).

Bifunctional domains could stably drive both activation and repression or could fluctuate between these activities over time. To distinguish between these possibilities, transcription driven by the bifunctional ARGFX tile 16 was quantified (FIG. 4B) at the minCMV promoter over 4 days and activation peaked at day 1 and then decreased over time (FIG. 14D). Intrigued by these dynamics, activation dynamics for ARGFX tile 16 and several other bifunctional domains (FOXO1, NANOG, and KLF7) recruited to a promoter of moderate strength (PGK) were profiled (FIGS. 4E-4F, 14E). Surprisingly, ARGFX tile 16 initially activated transcription at the PGK promoter from a low to a high state but then the cell population split into two subpopulations: activated (high) or repressed (off). Other domains (e.g., ARGFX tile 19 and FOXO1 tile 56) showed similar behavior at the minCMV and PGK promoters, initially activating and then decreasing transcription over time. They also contained overlapping regions for both activities. Several domains with bifunctional activities at the minCMV and pEF promoters did not significantly alter transcription when recruited to the PGK promoter, establishing that observed activities are promoter-dependent. For these domains, deletion scan measurements revealed independent regions for activation and repression (FIG. 13G, SEQ ID NOs: 25652-28198). In summary, some bifunctional tiles that independently activated and repressed different promoters are bifunctional even at a single promoter and can dynamically split a cell population into high- and low-expressing cells.

### Example 5

### Bifunctional Activating and Repressing Domains

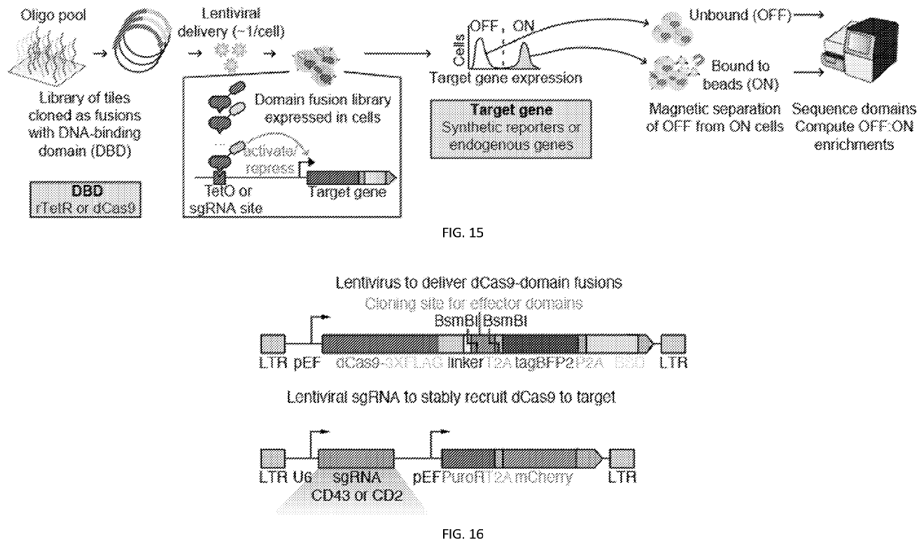

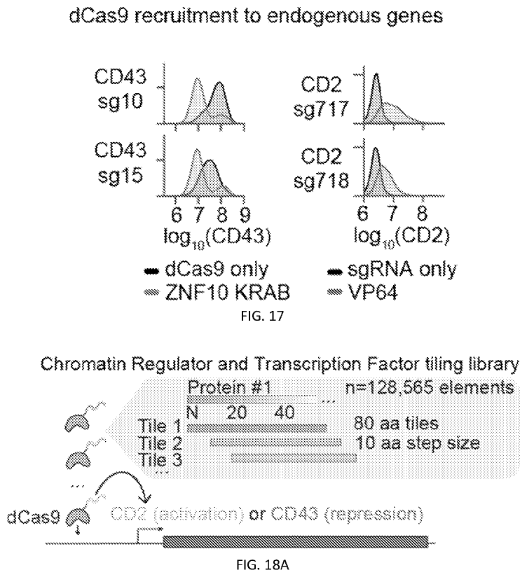

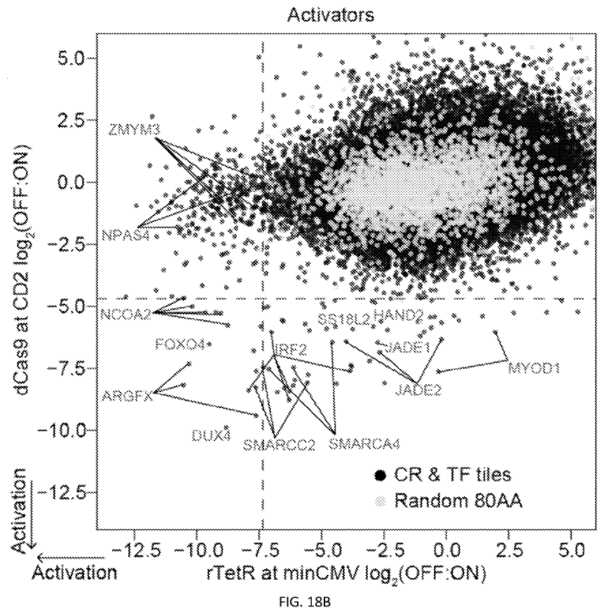

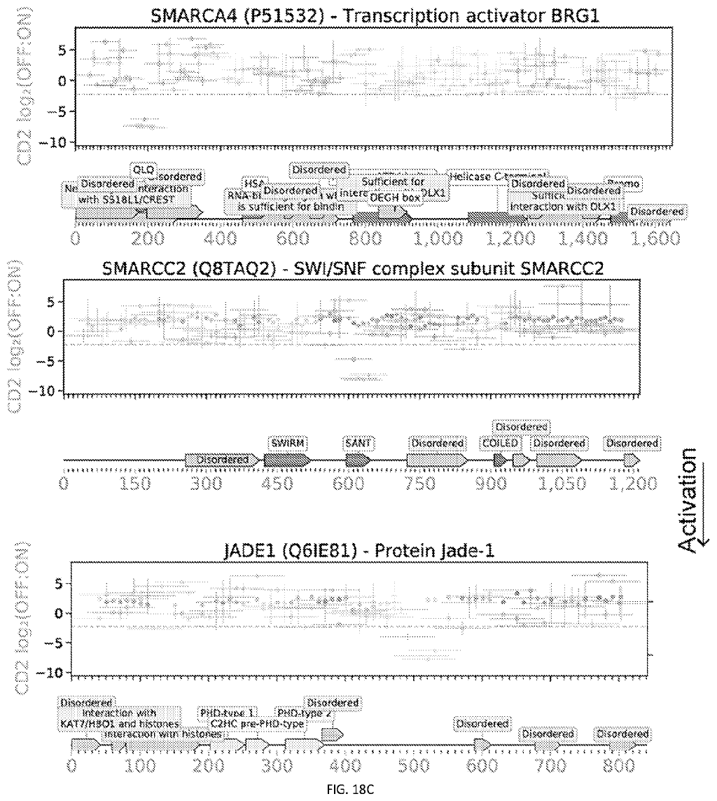

In order to extend the approach to endogenous loci, dCas9 was used to target the promoters of endogenous cell surface proteins (FIG. 15). Targeting surface proteins allowed use of fluorescent antibodies to immunostain cells, thus providing a way to monitor single-cell gene expression variability during individual recruitment assays by flow cytometry and to magnetically separate a large number of ON and OFF cells during HT-recruit (FIGS. 15 and 16). To study repressors, the highly expressed surface marker CD43 in K562 cells was targeted. First, either dCas9 alone or dCas9-KRAB were individually recruited from ZNF10 with sgRNAs targeting the CD43 transcriptional start site (TSS) and two sgRNAs, sg10 and sg15, were found for which repression depended on the KRAB repressor (FIG. 17). Similarly, sgRNAs were identified with which dCas9-VP64 could activate the lowly-expressed CD2 gene.

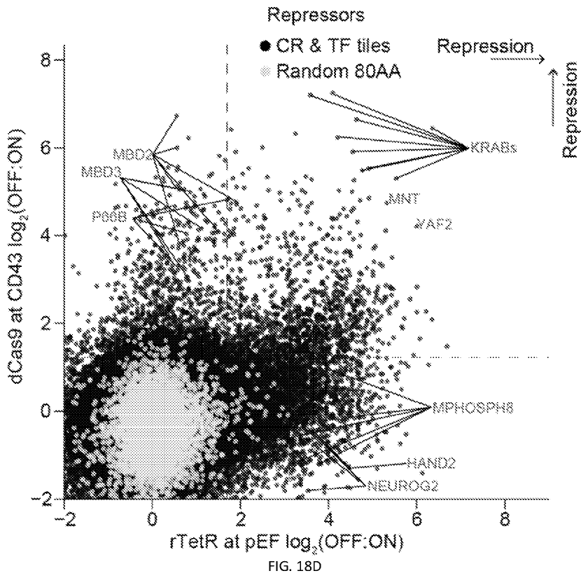

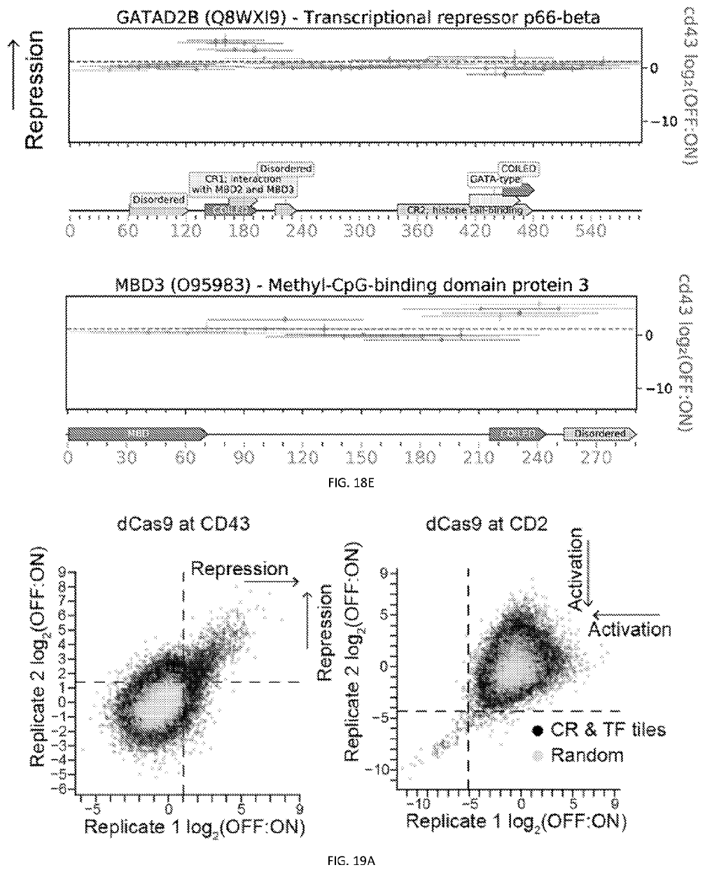

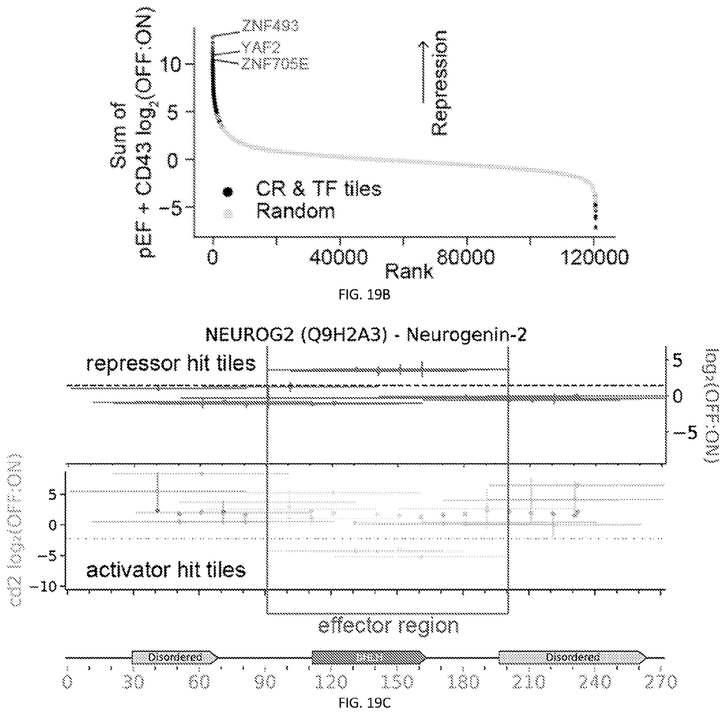

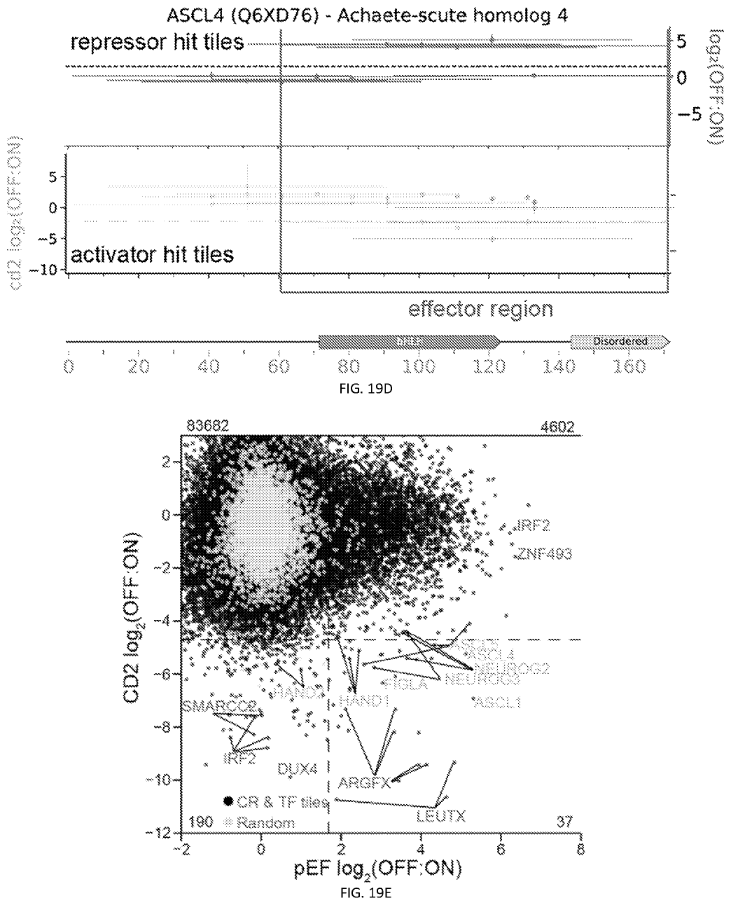

dCas9 recruitment to CD2 identified greater than 50 activator tiles that were not hits with rTetR at minCMV, including more HLH activators and SWI/SNF components (as with the Pfam library) and an unannotated region of the PHD proteins JADE1/2/3 (FIGS. 18A-C and 19A). A notably strong shared activator hit was the DUX4 C-terminus, which interacts with histone acetyltransferase P300. dCas9 recruitment to CD43 identified greater than 1000 repressor tiles that were not hits at pEF1a, including from more methyl-binding domain proteins (FIGS. 18D and 18E). The strongest shared repressors were KRAB domains (FIG. 19B). Meanwhile, 74% of proteins with a dual-function tile that activates CD2 (but not minCMV) and represses pEF1a were HLH proteins, and the higher resolution tiling data was used to map their dual-functioning region to the heterodimerizing HLH portion (and not the DNA binding basic portion) of their basic-HLH domains (FIGS. 19C-19E). Altogether, this represents a resource of transcriptional effectors, including from unannotated protein regions, that function on dCas9 and can enable campaigns to engineer transcription perturbations tools.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A synthetic transcription factor comprising one or more activator domains, one or more repressor domains, or a combination thereof fused to a heterologous DNA binding domain,
wherein at least one of the one or more activator domains or at least one of the one or more repressor domains comprises an amino acid sequence having at least 70% identity to any of SEQ ID NOS: 1-12567 and 28214-28404.

2. (canceled)

3. The synthetic transcription factor of claim 1, wherein at least one of the one or more activator domains or at least one of the one or more repressor domains comprises an amino acid sequence of any of SEQ ID NOS: 1-12567 and 28214-28404.

4. A synthetic transcription factor comprising one or more activator domains, one or more repressor domains, or a combination thereof fused to a heterologous DNA binding domain,
wherein at least one of the one or more activator domains or the one or more repressor domains comprises at least 10 contiguous amino acids of any of SEQ ID NOS: 1-12567 and 28214-28404.

5. The synthetic transcription factor of claim 1, wherein at least one of the one or more activator domains comprises an amino acid sequence having at least 70% identity to any of SEQ ID NOs: 31, 36, 111, 113, 153, 158, 165, 182, 184, 189, 224, 291, 311, 313, 352, 362, 367, 369, 375, 381, 407, 410, 415, 426, 430, 436, 472, 476, 478, 480, 483, 487-489, 494, 496, 498, 509, 512-517, 524, 526, 527, 530, 532, 533, 537, 541, 542, 545-547, 549, 552, 554, 557, 560-562, 565-568, 570-576, 578, 579, 580, 581, 582, 585, 587, 589, 590, 592, 595-598, 601, 603, 605, 607, 613, 617, 620, 622-624, 626, 627, 629, 630, 634-636, 639, 643, 646, 648, 651, 654, 658, 659, 662, 664, 666, 673, 675, 677, 678, 681, 684, 685, 686, 687, 689, 695, 696, 697, 699, 704, 705, 707-711, 713, 715, 716, 721, 723-725, 728, 729, 731-733, 735, 744, 746, 747, 753, 755, 760, 761, 764, 766-769, 773, and 775-984.

6. The synthetic transcription factor of claim 1, wherein at least one of the one or more activator domains comprises an amino acid sequence having at least 70% identity to any of SEQ ID NOs: 12568-17423.

7. (canceled)

8. The synthetic transcription factor of claim 1, wherein at least one of the one or more activator domains comprises one or more of SEQ ID NOs: 17424-17841.

9. The synthetic transcription factor of claim 1, wherein at least one of the one or more repressor domains comprises an amino acid sequence having at least 70% identity to any of SEQ ID NOs: 1036, 1054, 1055, 1069, 1120, 1144, 1182, 1183, 1200, 1208, 1314, 1318, 1366, 1402, 1417, 1442, 1516, 1518, 1543, 1598, 1627, 1655, 1665, 1667, 1670, 1706, 1710, 1711, 1735, 1738, 1742, 1747, 1748, 1752, 1756, 1763, 1777, 1783, 1786, 1789, 1793, 1794, 1808, 1811, 1822, 1831, 1838, 1839, 1854, 1859, 1862, 1865, 1866, 1869, 1870, 1872, 1875, 1883, 1889, 1891, 1893, 1901, 1902, 1905, 1907, 1910, 1912, 1913, 1914, 1915, 1916, 1922, 1923, 1927, 1930, 1934, 1940, 1944, 1946, 1948, 1951, 1952, 1956, 1957, 1968, 1969, 1972, 1987, 1992, 1994, 1996, 2004, 2007, 2010, 2017, 2022, 2029, 2033, 2041, 2042, 2043, 2048, 2050, 2051, 2053, 2057, 2064, 2095, 2107, 2112, 2119, 2123, 2128, 2131, 2139, 2150, 2157, 2160, 2163, 2176, 2182, 2188, 2190, 2192, 2193, 2194, 2205, 2206, 2207, 2208, 2211, 2212, 2213, 2216, 2218, 2221, 2224, 2227, 2231, 2232, 2239, 2245, 2246, 2254, 2262, 2263, 2265, 2271, 2274, 2275, 2277, 2278, 2282, 2283, 2288, 2292, 2295, 2296, 2298, 2302, 2312, 2313, 2316, 2320, 2321, 2323, 2324, 2325, 2334, 2338, 2341, 2348, 2361, 2364, 2365, and 2370-6094.

10. The synthetic transcription factor of claim 1, wherein at least one of the one or more repressor domains comprises an amino acid sequence having at least 70% identity to any of SEQ ID NOs: 17842-25651.

11. (canceled)

12. The synthetic transcription factor of claim 1, wherein the heterologous DNA binding domain is a programmable DNA binding domain or is part of an inducible DNA binding system.

13. The synthetic transcription factor of claim 1, wherein the heterologous DNA binding domain is derived from a Clustered Regularly Interspaced Short Palindromic Repeats associated (Cas) protein or a Transcription activator-like effectors (TALEs) domain.

14-15. (canceled)

16. A nucleic acid encoding a synthetic transcription factor of claim 1.

17-18. (canceled)

19. A cell comprising a synthetic transcription factor of claim 1 or one or more nucleic acids encoding thereof.

20-23. (canceled)

24. A composition comprising one or more synthetic transcription factors of claim 1 or one or more nucleic acids encoding thereof, or a cell comprising one or more synthetic transcription factors or one or more nucleic acids encoding thereof.

25. (canceled)

26. The system of claim 24, further comprising a guide RNA or a nucleic acid encoding a guide RNA.

27. (canceled)

28. A method of modulating the expression of at least one target gene in a cell, the method comprising introducing into the cell at least one synthetic transcription factor of claim 1 or a nucleic acid encoding thereof.

29. The method of claim 28, wherein the at least one target gene is an endogenous gene, an exogenous gene, or a combination thereof.

30. The method of claim 28, wherein the cell is in a subject and the method comprises administering the at least one synthetic transcription factor, nucleic acid, or composition or system comprising thereof to the subject.

31. (canceled)

32. The method of any of claim 28, wherein the gene expression of at least two genes is modulated.

33. A method for treating a disease or condition in a subject in need thereof, the method comprising: administering to the subject at least one synthetic transcription factor of claim 1 or a nucleic acid encoding thereof, or composition or system comprising thereof to the subject.

34. (canceled)

35. The method of claim 33, wherein the synthetic transcription factor alters the expression of a disease-related gene.

36-38. (canceled)
